(19) United States
(12) Patent Application Publication
(10) Pub. No.: US 2003/0119029 A1
(43) Pub. Date: Jun. 26, 2003

(54) Compositions and methods relating to novel benzodiazepine compounds and targets thereof

(75) Inventors: Gary D. Glick, Ann Arbor, MI (US); Anthony W. Opipari, Ann Arbor, MI (US)

Correspondence Address: MEDLEN & CARROLL, LLP, 101 Howard Street, Suite 350, San Francisco, CA, 94105, US

(73) Assignee: Regents of the University of Michigan, Ann Arbor, MI

(21) Appl. No.: 10/217,878

(22) Filed: Aug. 13, 2002

Related U.S. Application Data
(63) Continuation-in-part of application No. 09/767,283, filed on Jan. 22, 2001.
Application No. 09/767,283 is a continuation of application No. 09/700,101, filed on Nov. 8, 2000.
Application No. 09/700,101 is a 371 of international application No. PCT/US00/11599, filed on Apr. 27, 2000.
(60) Provisional application No. 60/131,761, filed on Apr. 30, 1999.
(60) Provisional application No. 60/165,511, filed on Nov. 15, 1999.
(60) Provisional application No. 60/191,855, filed on Mar. 24, 2000.
(60) Provisional application No. 60/312,560, filed on Aug. 15, 2001.
(60) Provisional application No. 60/313,689, filed on Aug. 20, 2001.

(51) Int. Cl. A61K 31/5513; C12Q 1/68; A61K 31/55; A01N 43/62; C12N 7/00; C12N 7/01; C12N 5/00; C12N 5/02

(52) U.S. Cl. 435/6; 514/221; 435/235.1; 435/325

(57) ABSTRACT

The present invention relates to novel chemical compounds, methods for their discovery, and their therapeutic use. In particular, the present invention provides benzodiazepine derivatives and methods of using benzodiazepine derivatives as therapeutic agents to treat a number of conditions associated with the faulty regulation of the processes of programmed cell death, autoimmunity, inflammation, and hyperproliferation, and the like.

This application is a Continuation in Part of U.S. patent application Ser. No. 09/767,283, filed Jan. 22, 2001, which is a continuation of U.S. patent application Ser. No. 09/700,101, filed Nov. 8, 2000, which is the National entry of PCTUS00/11599 filed Apr. 27, 2000, which claims priority to U.S. Provisional Application Serial No. 60/131,761, filed Apr. 30, 1999, to U.S. Provisional Application Serial No. 60/165,511, filed Nov. 15, 1999, and to U.S. Provisional Application Serial No. 60/191,855, filed Mar. 24, 2000. This application also claims priority to U.S. Provisional Application Serial No. 60/312,560, filed Aug. 15, 2001, to U.S. Provisional Application Serial No. 60/313,689, filed Aug. 20, 2001, and to U.S. Provisional Application Express Mail No.: EV092300423, filed Jul. 18, 2002. Each aforementioned application is specifically incorporated herein by reference in it entirety.

This invention was supported in part with NIH grants GM46831 and A147450. The United States government may have rights in this invention.

### FIELD OF THE INVENTION

The present invention relates to novel chemical compounds, methods for their discovery, and their therapeutic use. In particular, the present invention provides benzodiazepine derivatives and methods of using benzodiazepine derivatives as therapeutic agents to treat a number of conditions associated with the faulty regulation of the processes of programmed cell death, autoimmunity, inflammation, and hyperproliferation, and the like.

### BACKGROUND OF THE INVENTION

Multicellular organisms exert precise control over cell number. A balance between cell proliferation and cell death achieves this homeostasis. Cell death occurs in nearly every type of vertebrate cell via necrosis or through a suicidal form of cell death, known as apoptosis. Apoptosis is triggered by a variety of extracellular and intracellular signals that engage a common, genetically programmed death mechanism.

Multicellular organisms use apoptosis to instruct damaged or unnecessary cells to destroy themselves for the good of the organism. Control of the apoptotic process therefore is very important to normal development, for example, fetal development of fingers and toes requires the controlled removal, by apoptosis, of excess interconnecting tissues, as does the formation of neural synapses within the brain. Similarly, controlled apoptosis is responsible for the sloughing off of the inner lining of the uterus (the endometrium) at the start of menstruation. While apoptosis plays an important role in tissue sculpting and normal cellular maintenance, it is also the primary defense against cells and invaders (e.g., viruses) which threaten the well being of the organism.

Not surprisingly many diseases are associated with dysregulation of the process of cell death. Experimental models have established a cause-effect relationship between aberrant apoptotic regulation and the pathenogenicity of various neoplastic, autoimmune and viral diseases. For instance, in the cell mediated immune response, effector cells (e.g., cytotoxic T lymphocytes “CTLs”) destroy virus-infected cells by inducing the infected cells to undergo apoptosis. The organism subsequently relies on the apoptotic process to destroy the effector cells when they are no longer needed. Autoimmunity is normally prevented by the CTLs inducing apoptosis in each other and even in themselves. Defects in this process are associated with a variety of autoimmune diseases such as lupus erythematosus and rheumatoid arthritis.

Multicellular organisms also use apoptosis to instruct cells with damaged nucleic acids (e.g., DNA) to destroy themselves prior to becoming cancerous. Some cancer-causing viruses overcome this safeguard by reprogramming infected (transformed) cells to abort the normal apoptotic process. For example, several human papilloma viruses (HPVs) have been implicated in causing cervical cancer by suppressing the apoptotic removal of transformed cells by producing a protein (E6) which inactivates the p53 apoptosis promoter. Similarly, the Epstein-Barr virus (EBV), the causative agent of mononucleosis and Burkitt's lymphoma, reprograms infected cells to produce proteins that prevent normal apoptotic removal of the aberrant cells thus allowing the cancerous cells to proliferate and to spread throughout the organism.

Still other viruses destructively manipulate a cell's apoptotic machinery without directly resulting in the development of a cancer. For example, the destruction of the immune system in individuals infected with the human immunodeficiency virus (HIV) is thought to progress through infected CD4⁺ T cells (about 1 in 100,000) instructing uninfected sister cells to undergo apoptosis.

Some cancers that arise by non-viral means have also developed mechanisms to escape destruction by apoptosis. Melanoma cells, for instance, avoid apoptosis by inhibiting the expression of the gene encoding Apaf-1. Other cancer cells, especially lung and colon cancer cells, secrete high levels of soluble decoy molecules that inhibit the initiation of CTL mediated clearance of aberrant cells. Faulty regulation of the apoptotic machinery has also been implicated in various degenerative conditions and vascular diseases.

It is apparent that the controlled regulation of the apoptotic process and its cellular machinery is vital to the survival of multicellular organisms. Typically, the biochemical changes that occur in a cell instructed to undergo apoptosis occur in an orderly procession. However, as shown above, flawed regulation of apoptosis can cause serious deleterious effects in the organism.

There have been various attempts to control and restore regulation of the apoptotic machinery in aberrant cells (e.g., cancer cells). For example, much work has been done to develop cytotoxic agents to destroy aberrant cells before they proliferate. As such, cytotoxic agents have widespread utility in both human and animal health and represent the first line of treatment for nearly all forms of cancer and hyperproliferative autoimmune disorders like lupus erythematosus and rheumatoid arthritis.

Many cytotoxic agents in clinical use exert their effect by damaging DNA (e.g., cis-diaminodichroplatanim(II) cross-links DNA, whereas bleomycin induces strand cleavage). The result of this nuclear damage, if recognized by cellular factors like the p53 system, is to initiate an apoptotic cascade leading to the death of the damaged cell.

However, existing cytotoxic chemotherapeutic agents have serious drawbacks. For example, many known cytotoxic agents show little discrimination between healthy and diseased cells. This lack of specificity often results in severe side effects that can limit efficacy and/or result in early mortality. Moreover, prolonged administration of many existing cytotoxic agents results in the expression of resistance genes (e.g., bcl-2 family or multi-drug resistance (MDR) proteins) that render further dosing either less effective or useless. Some cytotoxic agents induce mutations into p53 and related proteins. Based on these considerations, ideal cytotoxic drugs should only kill diseased cells and not be susceptible to chemo-resistance.

One strategy to selectively kill diseased cells is to develop drugs that selectively recognize molecules expressed in diseased cells. Thus, effective cytotoxic chemotherapeutic agents, would recognize disease indicative molecules and induce (e.g., either directly or indirectly) the death of the diseased cell. Although markers on some types of cancer cells have been identified and targeted with therapeutic antibodies and small molecules, unique traits for diagnostic and therapeutic exploitation are not known for most cancers. Moreover, for diseases like lupus, specific molecular targets for drug development have not been identified.

What are needed are improved compositions and methods for regulating the apoptotic processes in subjects afflicted with diseases and conditions characterized by faulty regulation of these processes (e.g., viral infections, hyperproliferative autoimmune disorders, chronic inflammatory conditions, and cancers).

### SUMMARY OF THE INVNETION

The present invention relates to novel chemical compounds, methods for their discovery, and their therapeutic use. In particular, the present invention provides benzodiazepine derivatives and methods of using benzodiazepine derivatives as therapeutic agents to treat a number of conditions associated with the faulty regulation of the processes of programmed cell death, autoimmunity, inflammation, and hyperproliferation, and the like.

For example, the present invention provides methods for regulating cell death comprising the step of providing: target cells having mitochondria; an agent that binds to oligomycin sensitivity conferring protein; and exposing the cells to the agent under conditions such that the agent binds to the oligomycin sensitivity conferring protein so as to increase superoxide levels or alter ATP levels in the cells. In some of embodiments, the target cells are in vitro cells. In other embodiments, the target cells are in vivo cells. In still further embodiments, the target cells are ex vivo cells.

The present invention contemplates a number of target cells are suitable for with the methods and compositions of the present invention, for example, target cells include, but are not limited to, cancer cells, B cells, T cells, and granulocytes.

Preferred agents for use in the methods of the present invention are the benzodiazepine and benzodione derivatives disclosed herein. The present invention is not intended to be limited, however, to the benzodiazepine and benzodione derivatives disclosed herein. Indeed, in some additional embodiments, any agent that binds to the oligomycin sensitivity conferring protein (OSCP) portion of the mitochondrial ATP synthase complex and that is useful for treating any one or more of a number of conditions including, but not limited to, hyperproliferative, autoimmune, inflammatory disease, graft-versus-host disease, transplant rejection, cancer, and the like, is used in the methods of the present invention. The present invention is not limited to agents that bind the OSCP portion of mitochondrial ATP synthase. As described herein, a number of non-limiting mechanisms and therapeutic agents useful in the methods of the present invention are provided.

In particularly preferred embodiments, the methods of the present invention provide an effective amount (e.g., therapeutically useful) of BZ-432 to patient. The skilled artisan will appreciate the numerous formulations that may be employed (e.g., tablets, powders, salves, creams, oral suspensions, injectable formulations, etc.). The skilled artisan is also familiar with dosing considerations and with the regulatory and administrative steps that must be taken when administering pharmaceutical compositions to a patient. In preferred embodiments, contemplated patients include but are not limited to mammals. In particularly preferred embodiments the methods and compositions of the present invention are directed to, and optimized for, administration to humans.

The present invention further provides compositions (e.g., benzodiazepine or a benzodione derivative) and methods (e.g., administration) directed to increasing cell death in target cells.

The present invention also provides compositions (e.g., benzodiazepine or a benzodione derivative) and methods (e.g., administration) for inhibiting proliferation in cells comprising the steps of providing: proliferating target cells having mitochondria; an agent that binds to mitochondrial ATP synthase complex; and exposing the cells to the agent under conditions such that the agent binds to the mitochondrial ATP synthase complex so as to increase superoxide levels or alter ATP levels in the cells.

In some embodiments of the present invention, the contemplated agents of the present invention binds to the oligomycin sensitivity conferring protein such that superoxide levels in the treated cells/tissues increase. In some of these embodiments, the target cells are proliferating cells. In particularly preferred embodiments, therapeutic levels of Bz-423 are administered to patient.

In still further embodiments, the present invention provides pharmaceutical compositions comprising: a sufficient dose of an agent that binds to oligomycin sensitivity conferring protein so as to increase superoxide or alter ATP levels in cells of a subject exposed to the agent; and instructions for using the agent for treating a condition (e.g., cancer, proliferative diseases, autoimmune diseases, graft-versus-host disease, transplant rejection, and the like).

The present invention provides, in still further embodiment, pharmaceutical compositions comprising: a sufficient dose of an agent (e.g., benzodiazepine or a benzodione derivative) that binds to mitochondrial ATP synthase complex so as to increase superoxide or alter ATP levels in cells of a subject exposed to the agent; and instructions for using the agent for treating an autoimmune disease, a proliferative disease, or cancer. In some of these embodiments, a preferred agent comprises Bz-432.

Additional embodiments of the present invention provide methods for identifying agents useful for treating proliferative disease, autoimmune diseases, or cancer comprising: providing: mitochondrial ATP synthase complex; benzodiazepine or a benzodione derivative; a candidate agent; exposing the mitochondrial ATP synthase complex to the benzodiazepine or a benzodione derivative and the candidate agent; and comparing the binding of the benzodiazepine or a benzodione derivative and the candidate agent to the mitochondrial ATP synthase complex. The methods of the present invention are limited by the measure being observed. For instance, in some embodiments, the comparing comprises observing cell death, growth rate, or cell number in cells containing the mitochondrial ATP synthase complex. In other embodiments, the comparing comprises measuring superoxide levels in cells containing the mitochondrial ATP synthase complex. In still further embodiments, the comparing comprises measuring binding affinities of the benzodiazepine or a benzodione derivative and the candidate agent to the mitochondrial ATP synthase complex. Alternatively, other embodiments contemplate a comparing step comprising detecting the binding of the candidate agent to oligomycin sensitivity conferring protein.

Also provided are methods for identifying pharmaceutical agents, comprising: providing an agent that binds to mitochondrial ATP synthase complex so as to generate superoxide free radicals, alter ATP levels, initiate cell death, or alter cellular proliferation; chemically modifying the agent to generate a library of candidate pharmaceutical agents; and selecting one or more individual members of the library of candidate agents based on their increased ability to generate superoxide free radicals, initiate cell death, or alter cellular proliferation compared to the agent. Additionally, some embodiments further comprise the step of testing the one or more individual members of the library for toxicity in a tissue or animal.

The therapeutic methods disclosed herein may additionally comprise the step of submitting the one or more individual members of the library to a regulatory agency for approval as a commercial product (e.g., The U.S. Food and Drug Administration).

Yet other embodiments of the present invention provide methods for screening for agents that selectively induce cell death or inhibit the growth or proliferation of activated cells, comprising: providing: a first cell sample comprising at least one unactivated cell; a second cell sample comprising at least one unactivated cell; a third cell sample comprising at least one unactivated cell; an effective amount of an activating agent; and an effective amount of a candidate agent; an effective ratio and amount of the activating agent and the candidate agent; and contacting the first cell sample with the effective amount of the activating agent; contacting the second cell sample with the effective amount of the candidate agent; contacting the third cell sample with the effective ratio and amount of the activating agent and the candidate agent; comparing the level of cell death or cell number in the third cell sample to the level of cell death or cell number in the first cell sample and the second cell sample; and comparing the amount of cell death or growth inhibition in the third cell sample to the level of cell death or growth inhibition in the first cell sample and the second cell sample. Optionally some of these embodiments further comprise the step of selecting a candidate agent contacted to the third sample if the level of cell death or growth inhibition in the third cell sample is greater than the cell death in the first cell sample and the second cell sample. Suitable samples for use in these embodiments comprise B cells, T cells, granulocytes, cancer cells, and the like.

Activating agents suitable for use in the methods of the present invention include, but are not limited to, T cell ligand, BAFF ligand, TNF, Fas ligand (FasL), Toll ligand, APRIL, CD40 ligand, cytokines, chemokines, hormones, steroids, a B cell ligand, gamma irradiation, UV irradiation, an agent or condition that enhances cell stress, and antibodies that specifically recognize and bind cell surface receptors (e.g., anti-CD4, anti-CD8, anti-CD20, anti-BAFF, anti-TNF, anti-CD40, anti-CD3, anti-CD28, anti-B220, anti-Toll receptor, anti-APRIL receptor, anti-B cell receptor, anti-T cell receptor, and the like).

Additional embodiments of the present invention also provide methods for inhibiting induced cell death in an activated target cell (e.g., in vitro or in vivo activated target cells) by contacting the activated target cell with an effective amount of an agent (e.g., tacrolimus or the like) that inhibits the formation of superoxide in said activated target cell prior to mitochondrial permeability transition.

Some other embodiments of the present invention provide methods for screening for agents that selectively induce cell death or inhibit the growth or proliferation of activated cells, comprising: providing: a first cell sample comprising at least one unactivated cell; second cell sample comprising at least one unactivated cell; a candidate agent; and contacting the first cell sample with the candidate agent; and comparing the intracellular concentration of superoxide in first and second cells. Some of these methods further comprise the additional step of selecting the candidate agent contacted to the first cell sample if the intracellular concentration of superoxide is greater in the first cell sample than in the second cell sample. In still further embodiments, the present invention further provides in these methods the additional step of providing: an agent known to increase superoxide levels in treated unactivated cells; a third cell sample comprising at least one unactivated cell; a fourth cell sample comprising at least one unactivated cell; treating the third cell sample with the agent known to increase superoxide levels; treating the fourth cell sample with said with said agent known to increase superoxide levels and said selected candidate agent; and identifying whether or not the candidate agent synergistically increases superoxide levels with the agent known to increase superoxide levels by determining whether superoxide levels are higher in the treated fourth cell sample as compared to the treated third cell sample. In yet another embodiment, the present invention provides a pharmaceutical cocktail comprising the agent known to increase superoxide levels and the identified candidate agent.

### DESCRIPTION OF THE FIGURES

FIG. 1 shows cell death of (NZB×NZW)F₁ (“NZB/W”) splenocytes measured by permeability to propidium iodide (“PI”) after in vivo treatment (24 h) comparing Bz-423 (10 μM) to other benzodiazepine receptor ligands at the same concentration. Control=media, Bz=Bz-423, Naph=1-naphthol, Cz=clonazepam, Dz=diazepam, Cl-Dz=4′-chlorodiazepam, PK=PK 11195.

FIGS. 2A through 2H show that Bz-423 reduces autoimmune nephritis and splenic hyperplasia in NZB/W mice. Renal histopathology (400×) in control (FIGS. 2A and 2C) and treated animals (FIGS. 2B an 2D) after 12 wks of dosing identified by hemotoxilin and eosin (“H&E”) (FIGS. 2A and 2B) or immunofluoresent staining of IgG deposition (FIGS. 2C and 2D). Spleen sections from a control (FIG. 2E) and treated mouse (FIG. 2F) stained with anti-B220. Germinal centers (“GC”) from control (FIG. 2G) and treated (FIG. 2H) spleen sections (frozen) stained for simultaneous detection of B220 (background) and fragmented DNA (white spots).

FIGS. 3A and 3B show the effect of activation and co-stimulation on Bz-423-induced apoptosis. Data are expressed as percent of cells PI-positive at 24 h. FIG. 3A shows dose-response of Ramos cells to Bz-423 with (soluble anti-IgM) or without stimulation. FIG. 3B shows the specific killing of primary B cells in the presence of indicated stimuli and 4 μM Bz-423. White bars=anti-IgM, black bars=Bz-423, and gray bars=anti-IgM plus Bz-423.

FIGS. 4A through 4C show that Bz-423 increases superoxide which functions as an apoptotic signal in B cell receptor (“BCR”)-activated cells. Ramos cells were treated with anti-IgM alone (control, --) or with Bz-423 at the indicated concentrations (−). Inserts show effect of vitamin E. (FIG. 4A) Superoxide levels 1 hr after treatment with Bz-423. (FIG. 4B) PI staining after 24 h demonstrates hypodiploid DNA content consistent with apoptosis. (FIG. 4C) Interference contrast microscopy (400×) demonstrates similar apoptotic appearance of BCR-activated and un-activated cells treated with Bz-423.

FIGS. 5A through 5D show properties of Bz-423. (FIG. 5A) Structure of Bz-423 and inactive congeners. (FIG. 5B) Effect of Bz-423, 4′-chlorodiazepam (4-ClDz), PK11195, ΔNAP, and ΔOH on Ramos cell viability at 24 h determined by permeability to PI. (FIG. 5C) Morphology of cells treated for 24 h with vehicle, Bz-423 (10 μM), or Bz-423 (10 μM) plus z-VAD (100 μM) determined by interference contrast microscopy (400×). (FIG. 5D) After treatment as in (FIG. 5C), cells were analyzed by flow cytometry to determine DNA content. Panels A-D are representative of >5 separate determinations.

FIGS. 6A and 6B show mediators of Bz-423-induced apoptosis. (FIG. 6A) Ramos cells were incubated with vehicle or Bz-423 for 1 h. Fluorescence intensity increased above control (shaded histogram) with 5 and 10 μM Bz-423 (green and red histograms, respectively). (FIG. 6B) Time-course of changes detected in Ramos cells upon treatment with Bz-423 (10 μM). Data are presented as the percentage of cells with indicated response relative to time-matched vehicle controls. No changes in cells treated with vehicle during this time frame were observed. Inset—Cytochrome c release. Lane 1—purified cytochrome c (20 ng); Lane 2—cytosolic fraction isolated 5 h after incubation with vehicle; Lanes 3 and 4—cytosolic fractions isolated 1 and 5 h after incubation with Bz-423, respectively. Panels A and B represent data from >5 separate determinations.

FIGS. 7A through 7D show Bz-423 generates ROS in isolated mitochondria. Rat liver mitochondria were incubated with DCFH-DA and the desired agent, and the fluorescence intensity was monitored. These measurement were conducted in duplicate and repeated with four mitochondrial preparations. (FIG. 7A) Data obtained in state 3 respiration. The slopes of each curve after the induction phase (1200-1500 s) are 2.1, 1.5, 1.1 (×10⁻³±15%) for, antimycin A (0.5 μM), Bz-423 (10 μM), and vehicle, respectively. A greater slope corresponds to a higher level of ROS production. (FIG. 7B) Swelling of mitochondria in state 3 buffer. Only Ca²⁺ (400 μM) triggers the MPT pore, which results in swelling. (FIG. 7C) Data obtained in state 3 respiration using the S15 fraction. (FIG. 7D) Data obtained with mitochondria in state 4 respiration. (FIG. 7E) Representative micrographs (630×) of mitochondria stained with DHE (red) or with DIOC₃(6) (green) in states 3 and 4.

FIGS. 8A and 8B show regulating ROS preserves mitochondrial function and blocks Bz-423-induced killing. (FIG. 8A) After pre-incubating Ramos cells for 30 min with either FK506 (1 μM, blue), vitamin E (100 μM, red), MnTBAP (100 μM, green), or no inhibitor (black), Bz-423 (10 μM) was added to the cultures. White bars indicate control samples treated with vehicle alone. Using flow cytometry, DHE fluorescence (O₂⁻) was measured at 1 h, caspase activation at 5 h, ΔΨₘ (DiOC₆(3)) at 5 h, and PI permeability and hypodiploid DNA at 24 h. (FIG. 8B) Effect on cell viability of adding FK506 at times relative to the addition of Bz-423. The results in panels A and B represent >5 separate determinations.

FIG. 9 depicts disease progression analysis for MRL-lpr mice treated according to the methods described herein (solid line) as compared to controls (dotted line). The percentage of disease-free animals (y-axis) is plotted over time α-axis).

FIGS. 10A-10C depict footpad swelling in MRL-lpr mice treated according to the methods described herein (FIG. 10A) as compared to controls (FIG. 10B). FIG. 10C is a graphical analysis.

FIG. 11 is a bar graph depicting the efficacy of using benzodiazepine to kill D2 neuroblastoma cells in vitro.

FIG. 12 is a graph that shows that ovarian cancer cells are killed by application of benzodiazepine in vitro.

### DEFINITIONS

To facilitate an understanding of the present invention, a number of terms and phrases are defined below.

As used herein, the term “benzodiazepine” refers to a seven membered non-aromatic heterocyclic ring fused to a phenyl ring wherein the seven-membered ring has two nitrogen atoms, as part of the heterocyclic ring. In some aspects, the two nitrogen atoms are in 1 and 4 positions, as shown in the general structure below.

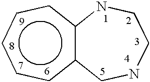

The benzodiazepine can be substituted with one keto group (typically at the 2-position), or with two keto groups, one each at the 2- and 5-positions. When the benzodiazepine has two keto groups, one each at the 2- and 5-positions, it is referred to as benzodiazepine-2,5-dione. Most generally, the benzodiazepine is further substituted either on the six-membered phenyl ring or on the seven-membered heterocyclic ring or on both rings by a variety of substituents. These substituents are described more fully herein.

As used herein, the term “substituted aliphatic” refers to an alkane possessing less than 10 carbons where at least one of the aliphatic hydrogen atoms has been replaced by a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic, etc.). Examples of such include, but are not limited to, 1-chloroethyl and the like.

As used herein, the term “substituted aryl” refers to an aromatic ring or fused aromatic ring system consisting of no more than three fused rings at least one of which is aromatic, and where at least one of the hydrogen atoms on a ring carbon has been replaced by a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, hydroxyphenyl and the like.

As used herein, the term “cycloaliphatic” refers to a cycloalkane possessing less than 8 carbons or a fused ring system consisting of no more than three fused cycloaliphatic rings. Examples of such include, but are not limited to, decalin and the like.

As used herein, the term “substituted cycloaliphatic” refers to a cycloalkane possessing less than 8 carbons or a fused ring system consisting of no more than three fused rings, and where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to, 1-chlorodecalyl and the like.

As used herein, the term “heterocyclic” refers to a cycloalkane and/or an aryl ring system, possessing less than 8 carbons, or a fused ring system consisting of no more than three fused rings, where at least one of the ring carbon atoms is replaced by oxygen, nitrogen or sulfur. Examples of such include, but are not limited to, morpholino and the like.

As used herein, the term “substituted heterocyclic” refers to a cycloalkane and/or an aryl ring system, possessing less than 8 carbons, or a fused ring system consisting of no more than three fused rings, where at least one of the ring carbon atoms is replaced by oxygen, nitrogen or sulfur, and where at least one of the aliphatic hydrogen atoms has been replaced by a halogen, hydroxy, a thio, nitro, an amino, a ketone, an aldehyde, an ester, an amide, a lower aliphatic, a substituted lower aliphatic, or a ring (aryl, substituted aryl, cycloaliphatic, or substituted cycloaliphatic). Examples of such include, but are not limited to 2-chloropyranyl.

As used herein, the term “linker” refers to a chain containing up to and including eight contiguous atoms connecting two different structural moieties where such atoms are, for example, carbon, nitrogen, oxygen, or sulfur. Ethylene glycol is one non-limiting example.

As used herein, the term “lower-alkyl-substituted-amino” refers to any alkyl unit containing up to and including eight carbon atoms where one of the aliphatic hydrogen atoms is replaced by an amino group. Examples of such include, but are not limited to, ethylamino and the like.

As used herein, the term “lower-alkyl-substituted-halogen” refers to any alkyl chain containing up to and including eight carbon atoms where one of the aliphatic hydrogen atoms is replaced by a halogen. Examples of such include, but are not limited to, chlorethyl and the like.

As used herein, the term “acetylamino” shall mean any primary or secondary amino that is acetylated. Examples of such include, but are not limited to, acetamide and the like.

The term “derivative” of a compound, as used herein, refers to a chemically modified compound wherein the chemical modification takes place either at a functional group of the compound or on the aromatic ring. Non-limiting examples of 1,4-benzodiazepine derivatives of the present invention may include N-acetyl, N-methyl, N-hydroxy groups at any of the available nitrogens in the compound. Additional derivatives may include those having a trifluoromethyl group on the phenyl ring.

As used herein, the term “subject” refers to organisms to be treated by the methods of the present invention. Such organisms preferably include, but are not limited to, mammals (e.g., murines, simians, equines, bovines, porcines, canines, felines, and the like), and most preferably includes humans. In the context of the invention, the term “subject” generally refers to an individual who will receive or who has received treatment (e.g., administration of benzodiazepine compound(s), and optionally one or more other agents) for a condition characterized by the dysregulation of apoptotic processes.

The term “diagnosed,” as used herein, refers to the to recognition of a disease by its signs and symptoms (e.g., resistance to conventional therapies), or genetic analysis, pathological analysis, histological analysis, and the like.

As used herein, the terms “anticancer agent,” or “conventional anticancer agent” refer to any chemotherapeutic compounds, radiation therapies, or surgical interventions, used in the treatment of cancer.

As used herein the term, “in vitro” refers to an artificial environment and to processes or reactions that occur within an artificial environment. In vitro environments can consist of, but are not limited to, test tubes and cell cultures. The term “in vivo” refers to the natural environment (e.g., an animal or a cell) and to processes or reaction that occur within a natural environment.

As used herein, the term “host cell” refers to any eukaryotic or prokaryotic cell (e.g., mammalian cells, avian cells, amphibian cells, plant cells, fish cells, and insect cells), whether located in vitro or in vivo.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro, including oocytes and embryos.

In preferred embodiments, the “target cells” of the compositions and methods of the present invention include, refer to, but are not limited to, lymphoid cells or cancer cells. Lymphoid cells include B cells, T cells, and granulocytes. Granulocyctes include eosinophils and macrophages. In some embodiments, target cells are continuously cultured cells or uncultered cells obtained from patient biopsies.

Cancer cells include tumor cells, neoplastic cells, malignant cells, metastatic cells, and hyperplastic cells. Neoplastic cells can be benign or malignant. Neoplastic cells are benign if they do not invade or metastasize. A malignant cell is one that is able to invade and/or metastasize. Hyperplasia is a pathologic accumulation of cells in a tissue or organ, without significant alteration in structure or function.

In one specific embodiment, the target cells exhibit pathological growth or proliferation. As used herein, the term “pathologically proliferating or growing cells” refers to a localized population of proliferating cells in an animal that is not governed by the usual limitations of normal growth.

As used herein, the term “un-activated target cell” refers to a cell that is either in the Gₒ phase or one in which a stimulus has not been applied.

As used herein, the term “activated target lymphoid cell” refers to a lymphoid cell that has been primed with an appropriate stimulus to cause a signal transduction cascade, or alternatively, a lymphoid cell that is not in Gₒ phase. Activated lymphoid cells may proliferate, undergo activation induced cell death, or produce one or more of cytotoxins, cytokines, and other related membrane-associated proteins characteristic of the cell type (e.g., CD8⁺ or CD4⁺). They are also capable of recognizing and binding any target cell that displays a particular antigen on its surface, and subsequently releasing its effector molecules.

As used herein, the term “activated cancer cell” refers to a cancer cell that has been primed with an appropriate stimulus to cause a signal transduction. An activated cancer cell may or may not be in the Gₒ phase.

An activating agent is a stimulus that upon interaction with a target cell results in a signal transduction cascade. Examples of activating stimuli include, but are not limited to, small molecules, radiant energy, and molecules that bind to cell activation cell surface receptors. Responses induced by activation stimuli can be characterized by changes in, among others, intracellular Ca²⁺, superoxide, or hydroxyl radical levels; the activity of enzymes like kinases or phosphatases; or the energy state of the cell. For cancer cells, activating agents also include transforming oncogenes.

In one aspect, the activating agent is any agent that binds to a cell surface activation receptor. These can be selected from the group consisting of a T cell receptor ligand, a B cell activating factor (“BAFF”), a TNF, a Fas ligand (FasL), a CD40 ligand, a proliferation inducing ligand (“APRIL”), a cytokine, a chemokine, a hormone, an amino acid (e.g., glutamate), a steroid, a B cell receptor ligand, gamma irradiation, UV irradiation, an agent or condition that enhances cell stress, or an antibody that specifically recognizes and binds a cell surface activation receptor (e.g., anti-CD4, anti-CD8, anti-CD20, anti-TACI, anti-BCMA, anti-TNF receptor, anti-CD40, anti-CD3, anti-CD28, anti-B220, anti-CD38, and-CD19, and anti-CD21). BCMA is B cell maturation antigen receptor and TACI is transmembrane activator and CAML interactor. (J. A. Gross et al., Nature, 404:995-999 [2000]; X. Laabi et al., EMBO J., 11:3897-3904 [19921]). Antibodies include monoclonal or polyclonal or a mixture thereof.

Examples of a T cell ligand include, but are not limited to, a peptide that binds to an MHC molecule, a peptide MHC complex, or an antibody that recognizes components of the T cell receptor.

Examples of a B cell ligand include, but are not limited to, a molecule or antibody that binds to or recognizes components of the B cell receptor.

Examples of reagents that bind to a cell surface activation receptor include, but are not limited to, the natural ligands of these receptors or antibodies raised against them (e.g., anti-CD20). RITUXIN (Genentech, Inc., San Francisco, Calif.) is a commercially available anti-CD 20 chimeric monoclonal antibody.

Examples of agents or conditions that enhance cell stress include heat, radiation, oxidative stress, or growth factor withdrawal and the like. Examples of growth factors include, but are not limited to serum, IL-2, platelet derived growth factor (“PDGF”), and the like.

As used herein, the term “effective amount” refers to the amount of a compound (e.g., benzodiazepine) sufficient to effect beneficial or desired results. An effective amount can be administered in one or more administrations, applications or dosages and is not limited intended to be limited to a particular formulation or administration route.

As used herein, the term “dysregulation of the process of cell death” refers to any aberration in the ability of (e.g., predisposition) a cell to undergo cell death via either necrosis or apoptosis. Dysregulation of cell death is associated with or induced by a variety of conditions, including for example, autoimmune disorders (e.g., systemic lupus erythematosus, rheumatoid arthritis, graft-versus-host disease, myasthenia gravis, Sjögren's syndrome, etc.), chronic inflammatory conditions (e.g., psoriasis, asthma and Crohn's disease), hyperproliferative disorders (e.g., tumors, B cell lymphomas, T cell lymphomas, etc.), viral infections (e.g., herpes, papilloma, HIV), and other conditions such as osteoarthritis and atherosclerosis.

It should be noted that when the dysregulation is induced by or associated with a viral infection, the viral infection may or may not be detectable at the time dysregulation occurs or is observed. That is, viral-induced dysregulation can occur even after the disappearance of symptoms of viral infection.

A “hyperproliferative disorder,” as used herein refers to any condition in which a localized population of proliferating cells in an animal is not governed by the usual limitations of normal growth. Examples of hyperproliferative disorders include tumors, neoplasms, lymphomas and the like. A neoplasm is said to be benign if it does not undergo, invasion or metastasis and malignant if it does either of these. A metastatic cell or tissue means that the cell can invade and destroy neighboring body structures. Hyperplasia is a form of cell proliferation involving an increase in cell number in a tissue or organ, without significant alteration in structure or function. Metaplasia is a form of controlled cell growth in which one type of fully differentiated cell substitutes for another type of differentiated cell. Metaplasia can occur in epithelial or connective tissue cells. A typical metaplasia involves a somewhat disorderly metaplastic epithelium.

The pathological growth of activated lymphoid cells often results in an autoimmune disorder or a chronic inflammatory condition. As used herein, the term “autoimmune disorder” refers to any condition in which an organism produces antibodies or immune cells which recognize the organism's own molecules, cells or tissues. Non-limiting examples of autoimmune disorders include rheumatoid arthritis, Sjögren's syndrome, graft versus host disease, myasthenia gravis, systemic lupus erythematosus (“SLE”), and the like.

As used herein, the term “chronic inflammatory condition” refers to a condition wherein the organism's immune cells are activated. Such condition is characterized by a persistent inflammatory response with pathologic sequelae. This state is characterized by infiltration of mononuclear cells, proliferation of fibroblasts and small blood vessels, increased connective tissue, and tissue destruction. Examples of chronic inflammatory diseases include, but are not limited to, Crohn's disease, psoriasis, chronic obstructive pulmonary disease, inflammatory bowel disease, multiple sclerosis, and asthma. Autoimmune diseases such as rheumatoid arthritis and systemic lupus erythematosus can also result in a chronic inflammatory state.

As used herein, the term “co-administration” refers to the administration of at least two agent(s) (e.g., benzodiazepines) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “toxic” refers to any detrimental or harmful effects on a cell or tissue as compared to the same cell or tissue prior to the administration of the toxicant.

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺, (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the terms “solid phase supports” or “solid supports,” are used in their broadest sense to refer to a number of supports that are available and known to those of ordinary skill in the art. Solid phase supports include, but are not limited to, silica gels, resins, derivatized plastic films, glass beads, cotton, plastic beads, alumina gels, and the like. As used herein, “solid supports” also include synthetic antigen-presenting matrices, cells, liposomes, and the like. A suitable solid phase support may be selected on the basis of desired end use and suitability for various protocols. For example, for peptide synthesis, solid phase supports may refer to resins such as polystyrene (e.g., PAM-resin obtained from Bachem, Inc., Peninsula Laboratories, etc.), POLYHIPE) resin (obtained from Aminotech, Canada), polyamide resin (obtained from Peninsula Laboratories), polystyrene resin grafted with polyethylene glycol (TENTAGEL, Rapp Polymere, Tubingen, Germany) or polydimethylacrylamide resin (obtained from Milligen/Biosearch, California).

As used herein, the term “pathogen” refers a biological agent that causes a disease state (e.g., infection, cancer, etc.) in a host. “Pathogens” include, but are not limited to, viruses, bacteria, archaea, fungi, protozoans, mycoplasma, prions, and parasitic organisms.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms which are gram negative or gram positive. “Gram negative” and “gram positive” refer to staining patterns with the Gram-staining process which is well known in the art. (See e.g., Finegold and Martin, Diagnostic Microbiology, 6th Ed., C V Mosby St. Louis, pp. 1³-15 [1982]). “Gram positive bacteria” are bacteria which retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red.

As used herein, the term “microorganism” refers to any species or type of microorganism, including but not limited to, bacteria, archaea, fungi, protozoans, mycoplasma, and parasitic organisms. The present invention contemplates that a number of microorganisms encompassed therein will also be pathogenic to a subject.

As used herein, the term “fungi” is used in reference to eukaryotic organisms such as the molds and yeasts, including dimorphic fungi.

As used herein, the term “virus” refers to minute infectious agents, which with certain exceptions, are not observable by light microscopy, lack independent metabolism, and are able to replicate only within a living host cell. The individual particles (i.e., virions) typically consist of nucleic acid and a protein shell or coat; some virions also have a lipid containing membrane. The term “virus” encompasses all types of viruses, including animal, plant, phage, and other viruses.

The term “sample” as used herein is used in its broadest sense. A sample suspected of indicating a condition characterized by the dysregulation of apoptotic function may comprise a cell, tissue, or fluids, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the terms “purified” or “to purify” refer, to the removal of undesired components from a sample. As used herein, the term “substantially purified” refers to molecules that are at least 60% free, preferably 75% free, and most preferably 90%, or more, free from other components with which they usually associated.

As used herein, the term “antigen binding protein” refers to proteins which bind to a specific antigen. “Antigen binding proteins” include, but are not limited to, immunoglobulins, including polyclonal, monoclonal, chimeric, single chain, and humanized antibodies, Fab fragments, F(ab′)2 fragments, and Fab expression libraries. Various procedures known in the art are used for the production of polyclonal antibodies. For the production of antibody, various host animals can be immunized by injection with the peptide corresponding to the desired epitope including but not limited to rabbits, mice, rats, sheep, goats, etc. In a preferred embodiment, the peptide is conjugated to an immunogenic carrier (e.g., diphtheria toxoid, bovine serum albumin (BSA), or keyhole limpet hemocyanin [KLH]). Various adjuvants are used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Köhler and Milstein (Köhler and Milstein, Nature, 256:495-497 [1975]), as well as the trioma technique, the human B-cell hybridoma technique (See e.g., Kozbor et al., Immunol. Today, 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

According to the invention, techniques described for the production of single chain antibodies (U.S. Pat. No. 4,946,778; herein incorporated by reference) can be adapted to produce specific single chain antibodies as desired. An additional embodiment of the invention utilizes the techniques known in the art for the construction of Fab expression libraries (Huse et al., Science, 246:1275-1281 [1989]) to allow rapid and easy identification of monoclonal Fab fragments with the desired specificity.

Antibody fragments that contain the idiotype (antigen binding region) of the antibody molecule can be generated by known techniques. For example, such fragments include but are not limited to: the F(ab′)2 fragment that can be produced by pepsin digestion of an antibody molecule; the Fab′ fragments that can be generated by reducing the disulfide bridges of an F(ab′)2 fragment, and the Fab fragments that can be generated by treating an antibody molecule with papain and a reducing agent.

Genes encoding antigen binding proteins can be isolated by methods known in the art. In the production of antibodies, screening for the desired antibody can be accomplished by techniques known in the art (e.g., radioimmunoassay, ELISA (enzyme-linked immunosorbant assay), “sandwich” immunoassays, immunoradiometric assays, gel diffusion precipitin reactions, immunodiffusion assays, in situ immunoassays (using colloidal gold, enzyme or radioisotope labels, for example), Western Blots, precipitation reactions, agglutination assays (e.g., gel agglutination assays, hemagglutination assays, etc.), complement fixation assays, immunofluorescence assays, protein A assays, and immunoelectrophoresis assays, etc.) etc.

As used herein, the term “immunoglobulin” or “antibody” refer to proteins that bind a specific antigen. Immunoglobulins include, but are not limited to, polyclonal, monoclonal, chimeric, and humanized antibodies, Fab fragments, F(ab′)₂ fragments, and includes immunoglobulins of the following classes: IgG, IgA, IgM, IgD, IbE, and secreted immunoglobulins (sIg). Immunoglobulins generally comprise two identical heavy chains and two light chains. However, the terms “antibody” and “immunoglobulin” also encompass single chain antibodies and two chain antibodies.

The term “epitope” as used herein refers to that portion of an antigen that makes contact with a particular immunoglobulin. When a protein or fragment of a protein is used to immunize a host animal, numerous regions of the protein may induce the production of antibodies which bind specifically to a given region or three-dimensional structure on the protein; these regions or structures are referred to as “antigenic determinants”. An antigenic determinant may compete with the intact antigen (i.e., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (i.e., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the terms “non-specific binding” and “background binding” when used in reference to the interaction of an antibody and a protein or peptide refer to an interaction that is not dependent on the presence of a particular structure (i.e., the antibody is binding to proteins in general rather that a particular structure such as an epitope).

As used herein, the term “modulate” refers to the activity of a compound (e.g., benzodiazepine compound) to affect (e.g., to promote or retard) an aspect of cellular function, including, but not limited to, cell growth, proliferation, apoptosis, and the like.

As used herein, the term “competes for binding” is used in reference to a first molecule (e.g., a first benzodiazepine derivative) with an activity that binds to the same substrate (e.g., the oligomycin sensitivity conferring protein in mitochondrial ATP synthase) as does a second molecule (e.g., a second benzodiazepine derivative or other molecule that binds to the oligomycin sensitivity conferring protein in mitochondrial ATP synthase, etc.). The efficiency (e.g., kinetics or thermodynamics) of binding by the first molecule may be the same as, or greater than, or less than, the efficiency of the substrate binding to the second molecule. For example, the equilibrium binding constant (K_{D}) for binding to the substrate may be different for the two molecules.

As used herein, the term “instructions for administering said benzodiazepine compound to a subject,” and grammatical equivalents thereof, includes instructions for using the compositions contained in a kit for the treatment of conditions characterized by the dysregulation of apoptotic processes in a cell or tissue. The term also specifically refers to instructions for using the compositions contained in the kit to treat autoimmune disorders (e.g., systemic lupus erythematosus, rheumatoid arthritis, graft-versus-host disease, myasthenia gravis, Sjögren's syndrome, etc.), chronic inflammatory conditions (e.g., psoriasis, asthma and Crohn's disease), hyperproliferative disorders (e.g., tumors, B cell lymphomas, T cell lymphomas, etc.), viral infections (e.g., herpes virus, papilloma virus, HIV), and other conditions such as osteoarthritis and atherosclerosis, and the like.

In some embodiments, the instructions further comprise a statement of the recommended or usual dosages of the compositions contained within the kit pursuant to 21 CFR §201 et seq. Additional information concerning labeling and instruction requirements applicable to the methods and compositions of the present are available at the Internet web page of the U.S. Food and Drug Administration (FDA).

In some embodiments, the instructions further comprise the statement of intended use required by the FDA in labeling in vitro diagnostic products. The FDA classifies in vitro diagnostics as medical devices and required that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use, including photographs or engineering drawings, where applicable; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; and 8) Any additional information regarding the in vitro diagnostic product requested that is necessary for the FDA to make a substantial equivalency determination. Additional information is available at the Internet web page of the U.S. FDA.

The term “test compound” refers to any chemical entity, pharmaceutical, drug, and the like, that can be used to treat or prevent a disease, illness, sickness, or disorder of bodily function, or otherwise alter the physiological or cellular status of a sample (e.g., the level of dysregulation of apoptosis in a cell or tissue). Test compounds comprise both known and potential therapeutic compounds. A test compound can be determined to be therapeutic by using the screening methods of the present invention. A “known therapeutic compound” refers to a therapeutic compound that has been shown (e.g., through animal trials or prior experience with administration to humans) to be effective in such treatment or prevention. In preferred embodiments, “test compounds” are agents that modulate apoptosis in cells.

As used herein, the term “third party” refers to any entity engaged in selling, warehousing, distributing, or offering for sale a test compound contemplated for administered with a benzodiazepine compound for treating conditions characterized by the dysregulation of apoptotic processes.

### GENERAL DESCRIPTION OF THE INVENTION

As a class of drugs, benzodiazepine compounds have been widely studied and reported to be effective medicaments for treating a number of disease. For example, U.S. Pat. Nos. 4,076823, 4,110,337, 4,495,101, 4,751,223 and 5,776,946, each incorporated herein by reference in its entirety, report that certain benzodiazepine compounds are effective as analgesic and anti-inflammatory agents. Similarly, U.S. Pat. No. 5,324,726 and U.S. Pat. No. 5,597,915, each incorporated by reference in its entirety, report that certain benzodiazepine compounds are antagonists of cholecystokinin and gastrin and thus might be useful to treat certain gastrointestinal disorders.

Other benzodiazepine compounds have been studied as inhibitors of human neutrophil elastase in the treating of human neutrophil elastase-mediated conditions such as myocardial ischemia, septic shock syndrome, among others. (See e.g., U.S. Pat. No. 5,861,380 incorporated herein by reference in its entirety). U.S. Pat. No. 5,041,438, incorporated herein by reference in its entirety, reports that certain benzodiazepine compounds are useful as anti-retroviral agents.

Despite the attention benzodiazepine compounds have drawn, it will become apparent from the description below, that the present invention provides novel benzodiazepine compounds and methods of using these compounds that are useful in treating a variety of disease characterized by the dysregulation of processes associated with cell death.

Experimental models have established a cause-effect relationship between derangement in the mechanism regulating apoptosis or necrosis and the pathenogenicity of various neoplastic, autoimmune, and viral diseases. (C. B. Thompson, Science, 267:1456-1462 [1995]). A well-defined example is the effect of aberrant, high-level expression of bcl-2 on lymphoma development. The bcl-2 oncogene was originally identified as the genetic element located at the t(14:18) chromosomal translocation breakpoint present in many B-cell follicular lymphomas. (S. J. Korsmeyer, Blood, 359:554-556 [1992]). Since that discovery, it has been reported that the bcl-2 gene product inhibits apoptosis induced by a variety of stimuli and that its oncogenic potential stems from its ability to derail apoptosis. (C. L. Sentman et al., Cell, 67:878-888 [1994]; and T. J. McDonnell S. J. and Korsmeyer, Nature, 349:254-256 [1991]).

Failed or reduced apoptosis has been reported to be associated with the development of human autoimmune lymphoproliferative syndrome as well as mouse models of this disease. MRL-lpr or gld mice develop lymphadenopathy, splenomegaly, nephritis and arthritis, as well as producing large quantities of autoantibodies. (P. L. Cohen, and R. A. Eisenberg, Annul. Rev. Immunol., 9:243-269 [1991]).

MRL-lpr mice carry loss of function mutations in the genes encoding FAS and Fas ligand, respectively. (M. Adachi et al., Proc. Natl. Acad. Sci. USA, 90:1756-1760 [1993]); T. Takakashi et al., Cell, 76:969-976 [1994]). FAS, a ubiquitously expressed cell surface receptor, normally generates an apoptotic response upon binding with Fas ligand. (N. Itoh, et al., Cell, 66:233-243 [1991]). In mice carrying these loss of function mutations, the disruption of FAS signaling renders T cells resistant to peripheral deletion by apoptosis. (H. Russell et al., Proc. Natl. Acad. Sci., USA 90:4409-4413). The inappropriate survival of these cells results in a pathologic accumulation of T and B cells evidenced by the neoplastic-like growth of lymphoid tissues and high-level autoantibody production. In humans, autoimmune lymphoproliferative syndromes shares similarities with the mouse phenotype including lymphadenopathy, splenomegaly, autoantibodies and autoimmune manifestations. Patients with this disease likewise have been reported to carry mutations in the FAS gene. (See, S. Nagata, J. Hum. Genet., 43:2-8 [1998]).

Benzodiazepine compounds are known to bind to benzodiazepine receptors in the central nervous system (CNS) and thus have been used to treat various CNS disorders including anxiety and epilepsy. Peripheral benzodiazepine receptors have also been identified, which receptors may incidentally also be present in the CNS. Benzodiazepines and related structures have pro-apoptotic and cytotoxic properties useful in the treatment of transformed cells grown in tissue culture. There is therapeutic potential for this class of agents against cancer and other neoplastic diseases. Two specific examples shown are neuroblastoma and ovarian cancer.

Neuroblastoma is the most common extracranial solid tumor found in children. Modern treatments, which include chemotherapy, radiation therapy and surgery, have not significantly reduced the mortality of metastatic neuroblastoma. Novel therapies are needed to improve survival of children with this disease. Some embodiments of the present invention provide compositions and methods that slow the growth of these tumors.

Likewise, ovarian cancer is difficult to treat due to chemoresistance shown by the patient to standard chemotherapy drugs. Treatment failures are usually attributed to the emergence of chemotherapy resistant cells. Some embodiments of the present invention provide benzodiazepine compounds that kill chemoresistant cancer cells (e.g., ovarian cancer cells).

### DETAILED DESCRIPTION OF THE INVENTION

The present invention relates to novel chemical compounds, methods for their discovery, and their therapeutic use. In particular, the present invention provides benzodiazepine derivatives and methods of using benzodiazepine derivatives as therapeutic agents to treat a number of conditions associated with the faulty regulation of the processes of programmed cell death, autoimmunity, inflammation, and hyperproliferation, and the like. Additionally, the present invention provides compositions and methods to regulate the processes of programmed cell death, autoimmunity, inflammation, and hyperproliferation, and the like, under pathological conditions.

Exemplary compositions and methods of the present invention are described in more detail in the following sections: I. Benzodiazepine derivative modulators of cell death; II. Benzodiazepine derivative modulators of cell growth and proliferation; III. Synthesis of exemplary benzodiazepine derivatives; IV. Pharmaceutical compositions, formulations, and exemplary administration routes and dosing considerations; V. Mitochondrial ATP synthase (mitochondrial F₀F₁ ATPase) activity modulators; and VI. Drug screens.

The practice of the present invention employs, unless otherwise indicated, conventional techniques of organic chemistry, pharmacology, molecular biology (including recombinant techniques), cell biology, biochemistry, and immunology, which are within the skill of the art. Such techniques are explained fully in the literature, such as, “Molecular cloning: a laboratory manual” Second Edition (Sambrook et al., 1989); “Oligonucleotide synthesis” (M. J. Gait, ed., 1984); “Animal cell culture” (R. I. Freshney, ed., 1987); the series “Methods in enzymology” (Academic Press, Inc.); “Handbook of experimental immunology” (D. M. Weir & C. C. Blackwell, eds.); “Gene transfer vectors for mammalian cells” (J. M. Miller & M. P. Calos, eds., 1987); “Current protocols in molecular biology” (F. M. Ausubel et al., eds., 1987, and periodic updates); “PCR: the polymerase chain reaction” (Mullis et al., eds., 1994); and “Current protocols in immunology” (J. E. Coligan et al., eds., 1991).

I. Benzodiazepine Derivative Modulators of Cell Death

In some embodiments of the present invention, the benzodiazepine compounds have the structure:

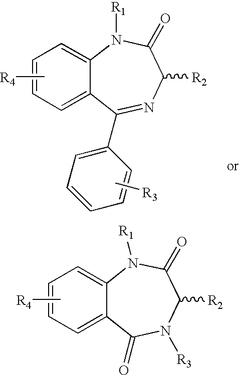

or its enantiomer, wherein, R₁ is aliphatic or aryl; R₂ is aliphatic, aryl, —NH₂, —HC(═O)—R₅, or a moiety that participates in hydrogen bond formation, wherein R₅ is aryl, heterocyclic, —R₆—NH—C(═O)—R₇ or —R₆—C(═O)—NH—R₇, wherein R₆ is an aliphatic linker of 1-6 carbons and R₇ is aliphatic, aryl, or heterocyclic; and each of R₃ and R₄ is independently hydrogen, hydroxy, alkoxy, halo, amino, lower-alkyl-substituted-amino, acylamino, hydroxyamino, an aliphatic group having 1-8 carbons and 1-20 hydrogens, aryl, or heteroaryl; or a pharmaceutically acceptable salt, prodrug or derivative thereof.

The cell death can be induced by necrosis, apoptosis or regulation of the FAS pathway. The conditions associated with the dysregulation of a process of cell death include but are not limited to: autoimmune diseases such as systemic lupus erythematosus, rheumatoid arthritis, Sjögren's syndrome, graft-versus-host-disease, and myasthenia gravis; chronic inflammatory conditions such as psoriasis, asthma, and Crohn's disease; hyperproliferative disorders or neoplasms such as a B-cell or a T-cell lymphomas; and other conditions such as osteoarthritis and atherosclerosis. Methods are also provided for using the benzodiazepine compounds to treat the conditions associated with the dysregulation of cell death, wherein the condition is induced by a viral infection. In addition, in some aspects, methods are provided to treat a viral infection by using the benzodiazepines of the present invention.

Methods are also provided to co-administer one or more additional agents with the benzodiazepines of the present invention, wherein such additional agents may include antineoplastic agents, immunosuppressants, anti-inflammatory agents, antiviral agents, or radiation.

The cell death to be achieved by the methods and compositions of this invention involve the cell or cells present in a tissue that are: autoimmunogenic or affected by an autoimmune disorder; inflammatory or affected by inflammation; hyperproliferative; viral-infected; atherosclerosed or osteoarthritic.

Assay and diagnostic methods are also provided to identify agents useful to treat a condition associated with dysregulation of the process of cell death in a subject wherein the ability of a potential candidate agent to induce cell death is assayed by contacting the dysregulated cell with a benzodiazepine compound. The assay includes maintaining the suitable cell or tissue preferably in a low serum.

Methods are also presented to prepare medicaments to treat a condition associated with dysregulation of the process of cell death in a subject, wherein the conditions, the affected cells or tissue and the benzodiazepine compounds are described as above. The invention also provides novel 1,4-benzodiazepine compounds having the structure:

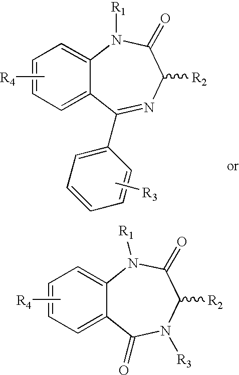

or its enantiomer, wherein, R₁ is aliphatic or aryl; R₂ is —NHC(═O)—R₅, wherein R₅ is aryl, heterocyclic, —R₆—NH—C(═O)—R₇ or —R₆—C(═O)—NH—R₇, wherein R₆ is an aliphatic linker of 1-6 carbons and R₇ is aliphatic, aryl, or heterocyclic; and each of R₃ and R₄ is independently hydrogen, hydroxy, alkoxy, halo, amino, lower-alkyl-substituted-amino, acylamino, hydroxyamino, an aliphatic group having 1-8 carbons and 1-20 hydrogens, aryl, or heterocyclic; or a pharmaceutically acceptable salt, prodrug or derivative thereof.

Exemplary target diseases and methods for identifying disease targets are presented below.

A. Target Diseases and Conditions

The present invention provides methods of treating conditions that are, in some embodiments, related in that they arise as the result of dysregulation of the normal processes of cell death (e.g., necrosis and/or apoptosis) in the cells or tissues of a subject. For the purpose of illustration only, such conditions include, but are not limited to, autoimmune disorders (e.g., systemic lupus erythematosus, rheumatoid arthritis, graft-versus-host disease, Sjögren's syndrome and myasthenia gravis); hyperproliferative disorders (e.g., B or T cell lymphoma, neuroblastoma, glioblastoma, chronic lymphocytic leukemia, breast cancer, prostate cancer, lung cancer, skin cancer, pancreatic cancer, colon cancer, melanoma, ovarian cancer, brain cancer, head and neck cancer, liver cancer, bladder cancer, non-small lung cancer, and cervical carcinoma); chronic inflammatory conditions (e.g., psoriasis, asthma, or Crohn's disease); other conditions such as osteoarthritis and atherosclerosis; and other conditions induced by DNA and/or RNA viral infections, wherein the viruses include, but are not limited to, herpes virus, papilloma virus and human immunodeficiency virus (HIV).

These disorders are treated by administering an effective amount of the benzodiazepine compounds described herein. The various benzodiazepine compounds are described more fully below. In preferred embodiments, these compounds are therapeutically effective on their own, and have few or no toxic effects when administered in large doses. Furthermore in some additional embodiments, as described in detail below, co-administration of these compounds with other agents provides an unexpected synergistic therapeutic benefit. In methods of co-administration, the claimed compounds are also useful in reducing deleterious side-effects of known therapeutic agents by decreasing the amount which must be administered to the subject.

The conditions which benefit from treatment with the compounds described herein appear to share the common etiology of dysregulation of the process of cell death. Normal apoptosis occurs via several pathways, with each pathway having multiple steps. The compositions and methods described herein are useful in treating dysregulated apoptosis regardless of the pathway or the step in the pathway where the dysfunction is occurring. In some embodiments, the conditions are caused by dysregulation of the FAS apoptotic pathway.

Similarly, the compounds are also useful in treating dysregulated necrosis regardless of the pathway or the step in the pathway where the dysfunction is occurring.

Dysregulation of the process of cell death is associated with many conditions.

In neoplasms, for example, normal cell death is inhibited, allowing hyperproliferative growth of cells. Aberrant functioning of this process can also result in serious pathologies including autoimmune disorders, viral infections, conditions induced by viral infections, neurodegenerative disease, and the like. The present invention provides methods of treating these and other conditions. While the present invention is not limited to any particular mechanism, nor to any understanding of the action of the agents being administered, it seems that the compounds described herein induce or promote cell death when this process is malfunctioning. The compounds of the present invention, however, are also useful for treating conditions not caused by defects in the apoptotic processes. For example, in certain viral infections, while there may not be any apoptotic defect, cell death may be promoted by inducing necrosis.

The condition to be treated is generally determined by noting the presence of symptoms in the subject or by noting phenotypic or genotypic changes in the cells of the subject, in particular, the inability of the cell to undergo apoptosis or necrosis. Phenotypic changes associated with the neoplastic state of a cell (e.g., a set of in vitro characteristics associated with a tumorigenic ability in vivo) include more rounded cell morphology, looser substratum attachment, loss of contact inhibition, loss of anchorage dependence, release of proteases, increased sugar transport, decreased serum requirement, expression of fetal antigens, etc. (See e.g., Luria et al., GENERAL VIROLOGY, 3rd edition, pp. 436-446 [1978] John Wiley & Sons, New York).

In some embodiments, treating cells or tissues refers to inducing cell death (wherein the cell death is either apoptotic or necrotic) in the cells or tissue which are causative (primary or distal) of the disorder being treated. For example, in hyperproliferative disorders, the method will treat the disorder by inducing apoptosis of the hyperproliferative cells, such as neoplastic cells. In this embodiment, reduction in tumor size or tumor burden is one means to identify that the object of the method has been met. In other aspects, treating encompasses restoration of immune function or regulation of immune dysfunction, as in autoimmune disorders and chronic inflammatory conditions. In further embodiments, treating encompasses ameliorating the symptoms associated with a particular disease (e.g., cachexia in cancer or HIV infection or inflammation in arthritis). In still further embodiments, prophylactic as well as therapeutic uses of the compounds and methods of this invention are intended.

In some embodiments, where the condition being treated is an autoimmune disease, the use of the methods disclosed herein reduce autoantibody production and lead to a decrease in inflammation and tissue destruction. Thus, a cell that is being treated may be the cell that itself is autoimmunogenic or is affected distally by an autoimmune reaction, wherein it is desirable to induce cell death in such a cell or in tissues containing such cells. Similarly, in the case of treating inflammatory conditions, the cell that is being treated may be the inflammatory cell itself or it may be distally affected by inflammation wherein it is desirable to induce cell death in such cells or tissues containing such cells and thus reduce inflammation.

In further embodiments, the cell being treated is a virally infected cell or a cell or tissue that previously has been infected. In some embodiments, successful therapies induce cell death and therefore a reduction in viral titer. This result is easily determined by assaying viral titer or by noting a reduction in cell number. It should be noted that in some instances it is desirable to induce cell death even among cells that do not have any viral remnants or other signs of viral infections at the time of treatment because a viral infection that occurred much earlier in time can cause disruption of cell death at a much later time.

Cell death may be assayed as described herein and in the art. In preferred embodiments, cell lines are maintained under appropriate cell culturing conditions (e.g., gas (CO₂), temperature and media) for an appropriate period of time to attain exponential proliferation without density dependent constraints. Cell number and or viability are measured using standard techniques, such as trypan blue exclusion/hemo-cytometry, or MTT dye conversion assay. Alternatively, the cell may be analyzed for the expression of genes or gene products associated with aberrations in apoptosis or necrosis.

In other embodiments of the present invention, the compounds of the present invention have antiviral activity independent of their efficacy to induce cell death. One aspect or method for inhibiting viral replication and/or propagation comprises contacting the virus with an effective amount of one or more compounds and/or compositions of the present invention. The contacting is conducted under suitable conditions to inhibit viral replication and/or propagation. In further embodiments, the methods comprises preventing viral infection and/or propagation in a cell or tissue by contacting the cell or tissue with an effective amount of the compounds and/or compositions as defined above. The contacting is conducted under suitable conditions to such that viral infection and/or propagation is inhibited.

It is contemplated that by inhibiting and reducing viral replication and proliferation, viral infectivity is also inhibited and reduced and the host cells are suitably treated for viral infection with the additional benefit that associated pathologies also are treated.

The viruses that are contemplated under the present methods include, but are not limited to, RNA and/or DNA viruses. By way of example only, such viruses are of herpes, non-herpes and retroviral origins. Major examples of human pathogens of the herpes virus family include herpes simplex viruses (HSV) 1, 2, and cercopithecine herpes virus 1 (B-virus); varicella-zoster; Epstein-Barr virus (EBV); Lymphocryptovirus; human herpes viruses 6-8 (HHV6-S); kaposi-associated herpes virus (KHV); herpes virus simiae, and human cytomegalovirus (HCMV). (See e.g., J. E. Gallant et al., J. Infect. Dis., 166:1223-1227 [1992]).

Animal pathogens of herpes viral origin include infectious bovine rhinotracheitis virus, bovine mammillitis virus, bovine leukemia virus (BLV) and cercopithecine herpes virus (B-virus), among others.

The human viruses of non-herpes origin include, but are not limited to, influenza viruses A, B and C; parainfluenza viruses-1,2, 3 and 4; adenovirus; reovirus; respiratory syncytial virus; rhinovirus; coxsackle virus; echo virus; rubeola virus; hepatitis viruses of the types Band C (HBV and HCV); and papovavirus.

The animal viruses of non-herpes origin include, but are not limited to, pseudorabies virus (PRV, of swine), equine rhinopneumonitis, coital exanthema viruses (varicella viruses); lymphocryptovirus; Marek's disease virus, Bovine Herpes virus-1 (BHV-1), herpes virus Pseudorabies virus (PRV).

The viruses of retroviral origin that are contemplated to be treatable by the compounds and compositions of this invention include, but are not limited to, human immunodeficiency viruses (HIV) of the types 1 and 2 and human lymphotropic 1 and 2 viruses (HTLV-I and II).

B. Methods of Identifying Potential Therapeutic Agents

Also provided herein are assays to identify potential agents to treat conditions associated with the dysregulation of the apoptotic or necrotic pathway. In some embodiments, the methods comprise contacting the dysregulated cell, i.e., a cell affected by the disorder (e.g., a tumor cell when the condition is hyperproliferative) or an immune cell (a neutrophil, basophil, eosinophil, monocyte, or lymphocyte) when the condition is a chronic inflammatory condition or an autoimmune disorder) with the potential therapeutic agent. In further aspects of the invention, control cells are further assayed with or without a benzodiazepine compound. The benzodiazepine compound may be a 1,4-benzodizepine compound as described herein. Cell death as compared to the control cells is also noted and compared. To identify potential therapeutic agents, appropriate assay conditions (e.g., incubation time, temperature, culture maintenance medium, etc.) are used and can be readily determined by one of skill in the art. Serum may be obtained from any commercial source, for example, fetal bovine serum from Gibco BRL (Gaithersburg, N. Mex.). The cells are cultured with the test agent for a sufficient amount of time for the test agent to affect apoptotic processes and/or necrosis. Following the appropriate incubation period, cell death is assayed by any means known, for example by MTT dye trypan exclusion. Novel cytotoxic agents are identified by their ability to induce the death of dysregulated cells versus cell death in control cells.

The present inventors have discovered that when the cells are maintained in low serum conditions, cytotoxicity is greatly exacerbated and the incubation time is reduced to about 2 hours or less. This is quite an unexpected result since under standard incubation conditions, which employ higher serum levels, the required incubation time is often several hours, approaching in some cases 24 hours or more. The term “low serum,” as used herein, refers to culture media containing less than about 10% per volume down to or equal to less than about 0.1% (v/v). It should be understood that within this range the concentration is flexible, and the applicants contemplate any possible subrange in increments of about 0.1% within this range, for example, less than or equal to about any of 0.1, 0.2, 0.3, 0.4, 0.5, 0.6, 0.7, 0.8, . . . 1.0, . . . 1.5, . . . 2.0, . . . 2.5, . . . 3.0, . . . 3.5, . . . 5%, etc., to about 9.9, and to about 10% of serum (% v/v).

Thus, in some embodiments, the benzodiazepines of this invention induce apoptosis in low serum as defined above.

In further embodiments, the compositions of the invention are further characterized and identified by their inability to bind either to a central benzodiazepine receptor or to bind with low affinity to a peripheral benzodiazepine receptor. However, in particularly preferred embodiments, the compositions and methods of the present invention do not target (e.g., selectively bind) either the central or peripheral benzodiazepine receptors. These compounds can be identified by using methods well-known in the art.

For example, the binding affinity of a benzodiazepine compound for a peripheral benzodiazepine receptor can be determined according to well-established methodology as described in H. Schoemaker et al., J. Pharm. Exp Ther., 225:61-69 (1983); and A. Doble et al., Brain Res. Bull., 18:49 1987).

Briefly, the method comprises comparing the potency of a benzodiazepine compound with that of a well-known high affinity binding agent such as 1-(2-chlorophenyl)-N-methyl-N-(-1, methylpropyl)-3-isoquinolinecarboxamide (PK11195), wherein the ability of the benzodiazepine compound to displace PK511195 from the peripheral benzodiazepine receptors in a competitive binding assay.

In any of the above assay methods, the benzodiazepine compound can be detectably labeled. Suitable detectable labels include, but are not limited, isotopes, chromophores, fluorophores, magnetic particles, high affinity binding partners (e.g., strepavidin/biotin), and antibodies, etc. Examples of isotope labeling include stable or radioactive isotopes of one or more atoms on the benzodiazepine molecule.

Methods for introducing detectable labels and for detecting the labels are well-known in the art. For example, the radioisotope label can be detected using special instrumentation, including electron spin resonance spectrometers. Stable isotopes can be detected using mass spectrometers, or magnetic resonance spectrometers. Fluorescent labels can be detected using fluorescent spectrometers. These instruments are commercially available and their operation is within the ordinary skill in the art.

The benzodiazepine compounds that can be used in the assay and diagnostic methods are described in greater detail below. It should be understood that all the compounds described therein, including the many general and specific embodiments, can be used in the assay and diagnostic methods.

II. Benzodiazepine Derivative Modulators of Cell Growth and Proliferation

The selectivity of many cytotoxic agents is limited and generally relies on the differential ability of diseased and healthy cells to tolerate and repair drug-induced cellular damage. However, developing cytotoxic therapies that exploit disease-specific targets remains challenging. For many diseases, suitable targets have not been identified, and in cases where targets exist (P. Huang and A. Oliff, Trends Cell. Biol., 11:343-248 [2001]), relatively few have been validated to the extent that it is known that blocking their function controls disease (D. W. Nicholson, Nature, 407:810-816 [2000]).

Diversity-oriented synthesis and phenotype screening (sometimes referred to as “chemical genetics”) have been advanced as robust methods for identifying bioactive compounds (S. L. Schreiber, Science, 287:1964-1969 [2000]). In these methods, arrays of molecules are synthesized and screened to identify lead compounds based directly on function rather than affinity for a target. Preferred embodiments, of the present invention provides compositions and methods that therapeutically target cytotoxic agents to diseased cells.

In one embodiment, the present invention provides selective compositions and methods for the therapeutic treatment of systemic lupus erythematosus (SLE) in a subject. SLE is characterized by a spectrum of antibodies that recognize self antigens. Autoantibodies are the products of B cells that escape peripheral tolerance. Autoantibody-antigen immune complexes deposit in the tissues of the heart, brain, lungs, and kidney, incite tissue destruction and impair organ function by activating complement and recruiting inflammatory cells. The kidney is an important target; at least 40% of all SLE patients have renal involvement, and lupus is associated with a mortality rate of 28% over 10 years.

Effective drugs to treat lupus include immunosuppressive lymphotoxic agents. Although effective, immunosuppressive drugs often induce severe side effects that account for a significant portion of lupus-related deaths. Agents with greater specificity toward disease-causing lymphocytes would clearly advance the treatment of SLE and related disorders. However, it is not yet possible to precisely identify the population of autoreactive B cells causing disease. Therefore, a specific therapy that only targets disease-causing cells does not exist. Despite its drawbacks, lymphocyte toxicity offers a reasonable basis by which to select new therapeutics.

The present invention provides methods for screening for candidate agents that selectively kill activated cells or inhibit cell growth or proliferation of an activated target cell by first contacting unactivated counterpart target cells (e.g., cells that have not been exposed to an activation stimulus) with varying amounts of the candidate agent and a separate sample of cells with equal varying amounts of buffer or an equivalent thereof, and selecting those candidate agents that increase the intracellular concentration of superoxide prior to the mitochondrial permeability transition (“MPT”) in the unactivated cell. As used herein, the term “an increase in intracellular superoxide prior to the MPT” is any statistically significant (p≦0.05) increase induced by a candidate agent compared to cells treated with buffer alone. Several methods to measure intracellular superoxide in cells are known in the art. One method employs hydroethidium as described by L. Benov et al., Free Radic. Biol. Med., 25:826-831 (1998). Several methods to measure the MPT in cells are known in the art. One method employs the dyes DIOC₆(3) and JC-1 as described by Zanzami et al. (1995).

Agents that produce increased superoxide concentration in the unactivated target cell prior to the MPT, are contacted with cells in the presence of micromolar amounts of FK506 or MnTBAP (or their equivalents) and the cells are assayed for levels of superoxide after treatment. These “inhibitors” can be added to the cells prior to or at the same time as the candidate compound. The FK506 screen identifies agents that, at their 50% effective concentrations (EC₅₀), have the change in superoxide concentration inhibited (to 75% inhibition) whereby FK506 is not acting solely by inhibiting the calcineurin pathway. (R. Zini et al., Life Sciences, 63(5):357-368 [1998]). The MnTBAP screen identifies agents that have the change in superoxide concentration inhibited (to >50% inhibition) with micromolar amounts of manganese (III) meso-tetrakis (4-benzoic acid) porphyrin (“MnTBAP”). In preferred embodiments, only after a candidate agent: 1) increases superoxide concentration in an unactivated cell prior to MPT; and 2) the increase in superoxide concentration in the unactivated cell is inhibited or reduced by FK506 or MnTBAP (or an equivalent) is the candidate agent selected for the third step of the screen. Both the second and third steps of the screen distinguishe the compounds having the preferred properties from those known in the art to also increase the intracellular concentration of superoxide prior to MPT. (See e.g., D. A Fennell et al., Br. J. of Cancer, 84(10):1397-1404 [2001]).

These agents are then further screened for their ability to inhibit the growth or proliferation (e.g., affect cell number), or induce death of an activated counterpart target cell to a greater extent than the combined effects of the activating agent alone and the candidate agent on unactivated cells.

In a separate embodiment, the screen is performed by first selecting an unactivated target, which can be a cultured cell or one obtained from a tissue biopsy. Serial dilutions from 0.01 nM through 100 mM of the compound or agent are made for testing. Serial dilutions are contacted with the cells by mixing with culture medium. Superoxide levels and the MPT in the unactivated target cells are measured for each dilution, and an EC₅₀ for each endpoint is determined. In an alternative aspect, the test cells receive serial dilutions (as above) of an inhibitor prior to contacting with the candidate agent or compound, and superoxide levels are then measured. The inhibition assay screens for compounds whose production of superoxide is inhibited by FK506 or MnTBAP or their equivalents at the agent's EC₅₀. Candidate agents that are inhibited by FK506 and MnTBAP (or their equivalents) are then assayed against counterpart activated target cells by making serial dilutions of the candidate agent cell (from 0.01 nM through 100 mM) and an activating agent (of a suitable amount) for the target which are then separately contacted with the unactivated target cells. An agent that inhibits growth or proliferation, or kills activated cells to a greater extent than the combined effects of the activating agent alone and the candidate compound on unactivated cells is a compound of this invention.

These candidate agents have the ability to selectively inhibit the growth or proliferation, or kill activated target cells and therefore, are useful for therapies to treat conditions or diseases associated with the pathological growth of the relevant target cell type in a subject. The agents also are useful to ameliorate the symptoms associated with the presence of pathologically growing activated target cells in a subject.

Agents or compounds identified by this screen have the general structure:

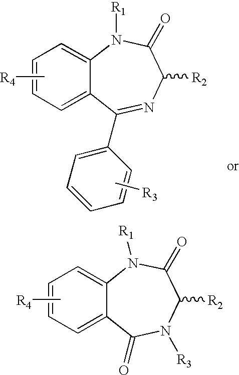

or their enantiomers, wherein R₁ is selected from the group consisting of an aliphatic, an aryl, a substituted aliphatic, and a substituted aryl and wherein R₂ is selected from the group consisting of a substituted aliphatic, a substituted aryl, a cycloaliphatic, a substituted cycloaliphatic, a heterocyclic, and a substituted heterocyclic NH₂, NHC(═O)—R₅ and any substituent that participates in hydrogen bond formation. R₅ is selected from the group consisting of an aryl, a heterocycle, and —R₆—NH—C(═O)—NH—R₇, wherein R₆ is an aliphatic linker of 1 to 6 carbons and R₇ is selected from the group consisting of an aliphatic, an aryl, and a heterocycle. Each of R₃ and R₄ may be the same or different and is selected from the group consisting of hydrogen, hydroxy, alkoxy, halo, amino, thio, nitro, lower-alkyl-substituted-amino, lower-alkyl-substituted-halo, acetylamino, hydroxyamino, an aliphatic group having 1 to 8 carbons, aryl, substituted aryl, cycloaliphatic, substituted cycloaliphatic or heterocyclic, a ketone, an aldehyde, an ester and an amide. In one aspect: R₁ is H, R₄ is a halogen, R₃ is hydroxyl or halogen, and R₂ is an aromatic or heterocycle. Unless specifically recited, all substituents are substituted or unsubstituted. Pharmaceutically acceptable salts of such compounds are further provided by this invention.

More specific examples of compounds identified by the screen are compounds having the structures:

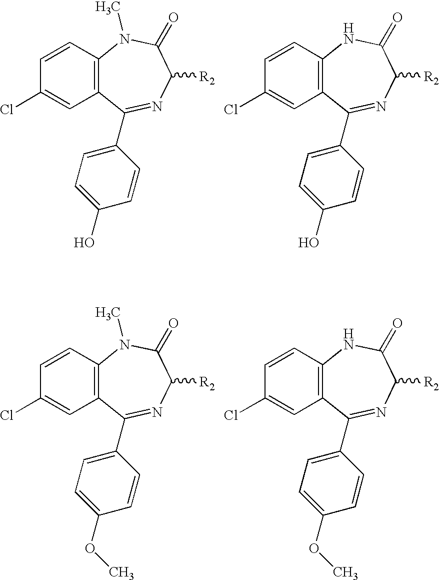

wherein R₂ is selected from the group consisting of:

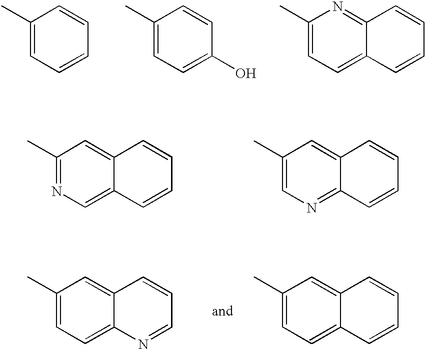

and dimethylphenyl (all isomers) and ditrifluoromethyl (all isomers), and

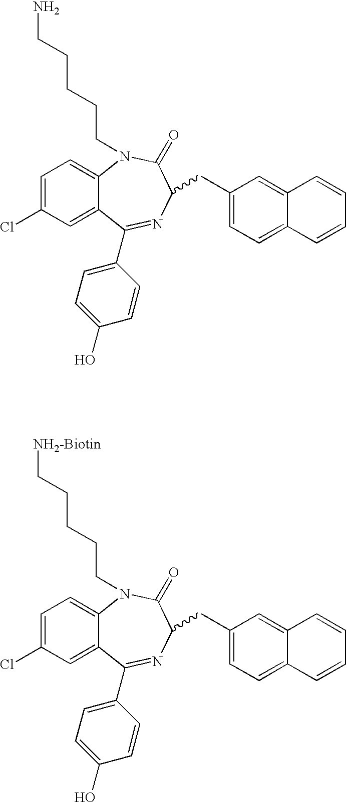

This invention also provides the compound Bz-423.

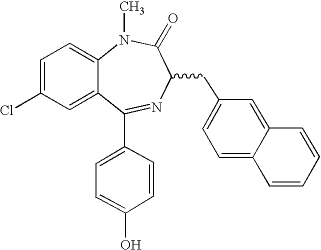

Bz-423 differs from benzodiazepines in clinical use by the presence of a hydrophobic substituent at C-3. This substitution renders binding to the peripheral benzodiazepine receptor (“PBR”) weak (K_{d} ca. 1 μM) and prevents binding to the central benzodiazepine receptor so that Bz-423 is not a sedative.

The agents or compounds that possess the above noted three properties are examples of agents of this invention. The compounds of this invention were identified by this screen and confirmed in an animal model. Further provided by this invention are the agents identified by this screen.

Although Applicants utilized this screen for small molecules that possess the desired properties, this invention encompasses other therapeutic modalities that are identified using this screen, e.g., polynucleotides or polypeptides, and thus are intended within the term “agents.”

The invention also provides methods for selectively killing and/or inhibiting cell growth or proliferation of activated target cells by contacting a target cell with an effective amount of an agent of this invention. Further provided are methods for selectively inducing cell death of a target cell in either its activated or un-activated state by contacting the target cell with an effective amount of an activating agent and an effective amount of an agent of this invention.

Also provided by the invention are methods for inhibiting cell death of a target cell by contacting the target cell with an effective amount of an agent that inhibits the formation of superoxide in the target cell prior to the cells' mitochondrial permeability transition, e.g., FK506 or its equivalent. These methods ameliorate the symptoms of neurodegenerative diseases such as Alzheimers and ischemia reprofusion injury, e.g., neuromotor problems and the like, and stroke, respectively.

The above methods of this invention can be practiced in vivo or in vivo. When practiced in vivo, the method provides a convenient animal model to confirm biological efficacy of agents identified by the screen of this invention.

Further provided is a method for selectively inhibiting the pathology of activated target cells in a subject in need of such therapy by administering to the subject an effective amount of an agent of this invention. Administration of an effective amount of the agents of this invention also serves to ameliorate the symptoms associated with the presence of pathologically growing activated target cells or to treat diseases associated with their presence in a subject. Suitable subjects include, but are not limited to a non-Hodgkin's lymphoma patient, a chronic lymphocytic leukemia patient, a cutaneous T cell leukemia patient, a patient with an autoimmune disorder, or a cancer patient (solid tumors, lymphomas, leukemias). Therapeutic compounds for use in these methods include the compounds described herein as well as those provided by PCT/US00/00578.

A method for selectively inhibiting the pathological growth of unactivated target cells in a subject in need of such therapy is further provided by this invention. An effective amount of an agent that selectively activates the target cells and an effective amount of an agent of this invention are administered to the subject. Administration of agents of the present invention can be simultaneous or sequential.

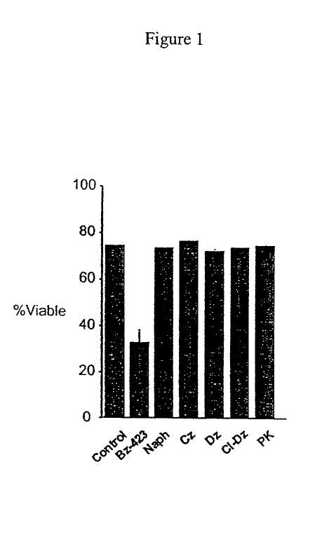

In one embodiment of the present invention, Bz-423 was selected from a 1,4-benzodiazepine library based on its ability to induce lymphoid cell death in vivo. Comparison of Bz-423 with other benzodiazepines and ligands of the peripheral benzodiazepine receptor reveals that Bz-423 has unique cytotoxicity. (FIG. 1). The activity of Bz-423 against NZB/W lymphocytes is concentrated on B cells such that treatment kills twice as many B cells as T cells. In some embodiments, the potential in vivo lymphotoxicity of Bz-423 was tested by administering (60 mg/kg/d for 7 d) of Bz-423 to autoimmune NZB/W and normal BALB/c mice. After treatment, splenic lymphocytes were analyzed for evidence of cell death. In the NZB/W mice, lymphocyte viability was decreased and B cell apoptosis increased (Table 1). Table 1 shows splenocyte viability (PI) and lineage specific apoptosis (TUNEL) after 7 days. By contrast, Bz-423 neither decreased viability nor increased apoptosis of BALB/c splenic lymphocytes. Thus, Bz-423 specifically effects B cells from the autoimmune mice.

TABLE 1

| | Cell | Apoptotic | Apoptotic |
|---|---|---|---|
| | Viability | B cells | T cells |
| Control | 85 ± 1 | 25 ± 2 | 12 ± 1 |
| Bz-423 | 81 ± 1 | 32 ± 2 | 12 ± 3 |
| | p = 0.006 | p = 0.0237 | p = 0.27 |

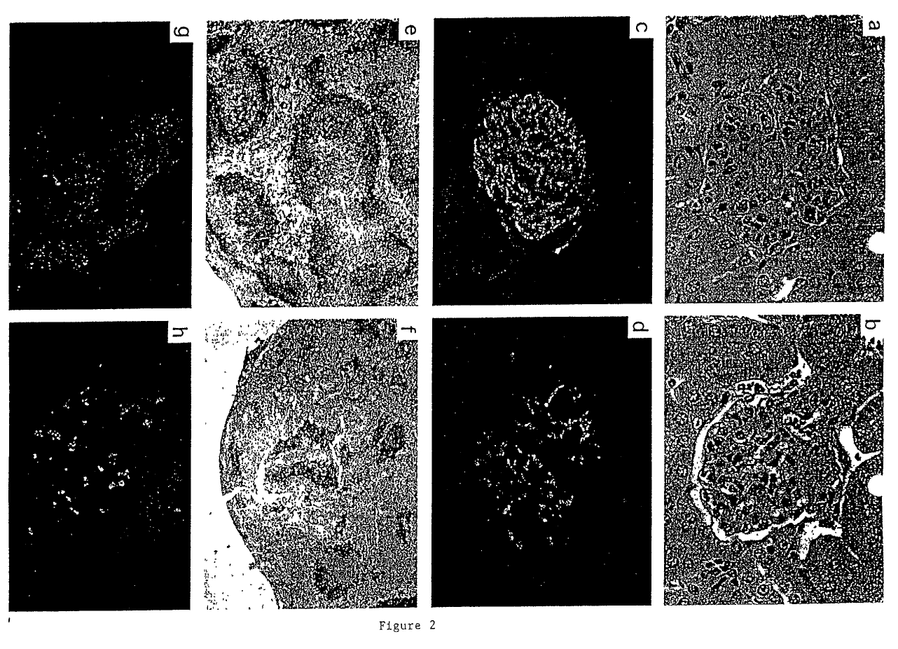

Given the selective action on autoimmune B cells, further studies were made to determine if Bz-423 affects the progression of autoimmune nephritis. For example, female NZB/W mice were given Bz-423 (60 mg/kg), or vehicle every other day from 6.5 to 9.5 months of age. Control mice developed diffuse, proliferative glomerulonephritis with expansion of all cellular elements and occasional wire-loop formation resulting in an average histopathologic score of 3+. (FIG. 2A). In contrast, mice dosed with Bz-423 had milder nephritic changes with an average score of 1+(p=0.002) (FIG. 2B). Bz-423 treated mice also had less glomerular IgG (FIGS. 2C and 2D) (IgG, p=0.002; C3, p=0.034). At the time of sacrifice, 83% of control mice had abnormally high BUN levels (≧30 mg/dL), compared to 27% of the treatment group (chi-square: p=0.001). A strong correlation was identified between the histological score for nephritis and proteinuria (>100 mg/dL; p=0.001) or elevated BUN (p=0.039). In aggregate, these measurements indicate less disease in treated animals compared to controls in some embodiments.

In still further embodiments, NZB/W mice with disease-related lymphoid hyperplasia characterized by the pathologic expansion of GC B cells were used in further studies. Flow cytometric measurements revealed that Bz-423 reduced the fraction of B cells in the spleen (49%±3 vs. 58%±3 in controls; p=0.05) with no statistical change in the fractional representation of T cells. Immunohistochemical staining demonstrated that this decrease resulted from a specific effect on GC B cells (FIGS. 2E and 2F). Mice treated with Bz-423 had fewer GCs relative to control mice (17±5 vs. 10±7 per 10 mm², p=0.01) and the GCs present in the drug group were almost 50% smaller than those present in the control animals (p=0.01). Furthermore, Bz-423-treated animals had increased TUNEL-positive B cells (ca. 3+ vs. 1+, p=0.038) that were predominantly concentrated in GCs (FIGS. 2G and 2H). This observation indicates that the decrease in GC size and number results from apoptosis within this compartment in some embodiments.

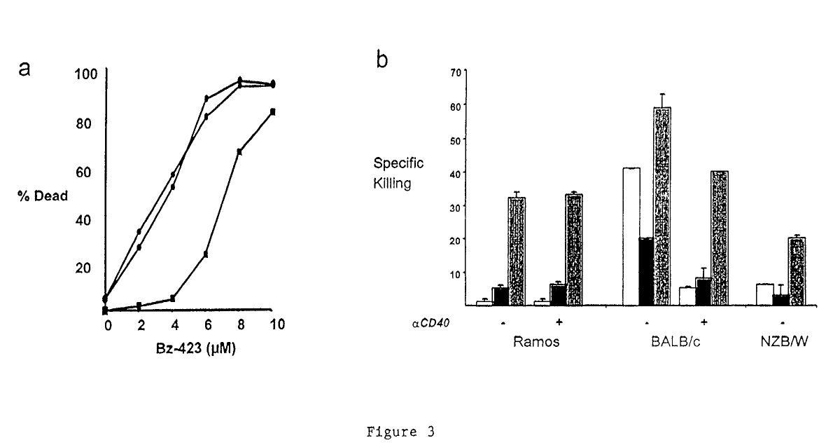

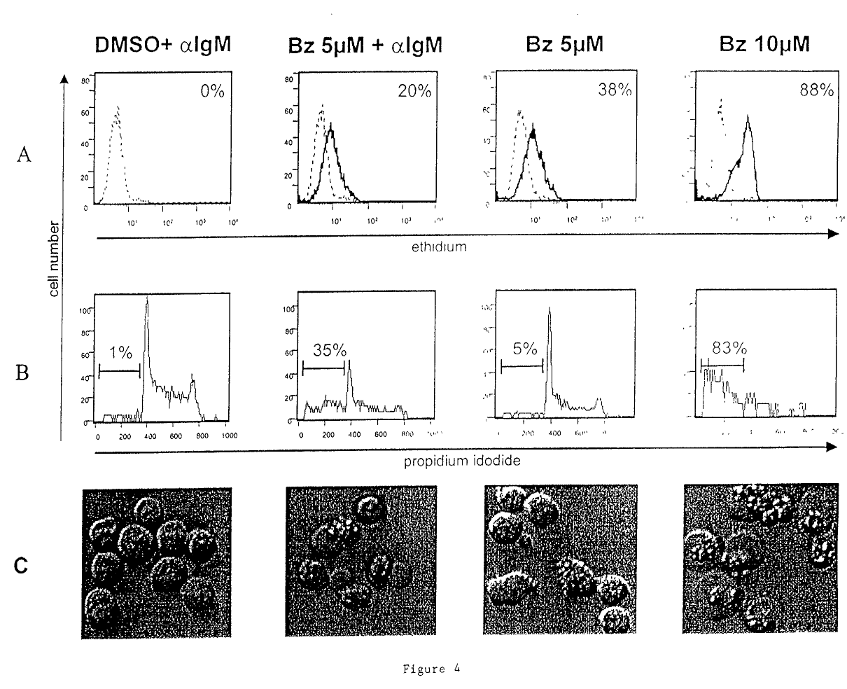

GC B cells require B cell receptor (BCR) stimulation for development and survival, a property that distinguishes them from other mature cells. To account for the selective reduction of GC in treated animals whether activation via BCR stimulation facilitates killing by Bz-423 was investigated. To test this hypothesis, two models of BCR stimulation were used that differ with respect to the degree of receptor cross-linking. In the first, Ramos cells were treated with soluble anti-IgM Fab₂ to provide a modest BCR signal that is itself insufficient to induce apoptosis. Ramos cells were utilized here because they display surface markers characteristic of GC cells, demonstrate a response to BCR ligation characteristic of mature B cells, and survive in culture with little spontaneous death. Soluble anti-IgM Fab₂, that alone fails to induce death, sensitizes to Bz-423 (FIG. 3A). In fact, treatment of activated Ramos cells with Bz-423 results in a synergistic, supra-additive death response. Stimulatory antibody directed against CD40, a signal that abrogates anti-IgM activation-induced cell death (AICD), offers no protection against Bz-423 alone, or anti-IgM sensitization to Bz-423 (FIG. 3B).

In a second activation model of BCR stimulation, primary NZB/W B cells were incubated with immobilized, whole anti-IgM which extensively cross-links BCRs and Fc receptors and provokes AICD in normal immune B cells. Consistent with previous reports, NZB/W B cells are resistant to apoptosis induced by receptor cross-linking compared to normal immune B cells. Nevertheless, stimulation with anti-IgM in the presence of Bz-423 kills NZB/W cells. While co-stimulation of CD40 completely blocks AICD in BALB/c cells, it provides little protection against Bz-423 alone, or death in the synergistic conditions. These results show that irrespective of the degree of receptor cross-linking, activated cells are more sensitive to Bz-423 in some embodiments.

The signals and markers associated with apoptosis in BCR simulated and un-activated Ramos cells were compared. With respect to morphology, treatment of both activated and unactivated cells results in cytoplasmic vaculization, nuclear condensation, and plasma membrane blebbing (FIG. 4C).

The dependence of sensitized death on pro-apoptotic signals that function in B cells was also tested. Chelating extracellular calcium with 1,2-bis(2-aminophenoxy)ethane-N,N,N′,N′-tetraaceticacid (BAPTA) minimally reduced non-sensitized death while substantially protecting sensitized cells (7% vs. 71% inhibition, respectively). The effect of BAPTA is unrelated to its ability to blunt the rapid BCR-induced calcium flux because it is protective even when added 1 hour after stimulation. Inhibition of caspase activity with z-VAD (un-activated: 31% vs. activated: 86%), the mitochondrial permeability transition (MPT) with cyclosporin (CsA) (un-activated: 9% vs. activated: 54%) and protein synthesis with cyclohexamide (un-activated: 19% vs. activated: 64%) only protected sensitized cells. Inhibiting calcineurin (with FK506 10 nM) was not protective in either condition. The antioxidants vitamin E and superoxide (O₂⁻)-specific MnTBAP each protected activated and unactivated cells to a similar extent (˜80%) suggesting that O₂⁻ is an essential event in Bz-423-induced signaling. These experiments show that high concentrations of Bz-423 generates superoxide which kills cells independently of these other mediators of apoptosis.

O₂⁻ in activated and un-activated cells using hydroethidium which is a selective indicator of O₂⁻, was also measured. O₂⁻ increases in both un-activated and activated cells within 1 hour of exposure to Bz-423 (FIG. 4A) which is prior to the MPT. MnTBAP and vitamin E reduced O₂⁻ levels, but z-VAD, BAPTA and CsA did not. The amount of O₂⁻ increases with increasing concentration of Bz-423, and in the absence of BCR stimulation, correlates with cell death measured at 24 h. However, for a given concentration of Bz-423, BCR stimulation does not increase O₂⁻ relative to un-activated cells (FIGS. 4A-4C). Hence, the role of superoxide in cell death differs in activated cells. BCR activated cells are killed by lower concentration of Bz-423 through a mechanism in which BCR cross-linking sensitizes cells to O₂⁻, gene expression, caspase, and mitochondria-dependent processes to occur.

These results demonstrate that in some preferred embodiments, the element(s) of the BCR response render B cells vulnerable to killing by low concentrations of Bz-423. Accordingly, in preferred embodiments, the selective toxicity of low concentrations of Bz-423 to BCR activated B cells provides a mechanism for this compound to target the expanded GC population based on their unique dependence on BCR stimulation. Although an understanding of the mechanism is not necessary to practice the present invention and the present invention is not so limited, it is contemplated that Bz-423 compensates for either of the two pathogenic mechanisms proposed to support persistent GCs and autoreactive B cells, by acting in concert with either modest or robust BCR cross-linking resulting in a supra-additive death response. Thus, the same stimulus that supports autoimmune pathogenesis provides a selective therapeutic target for some diseased cells. In human SLE, moreover, hyperactivated B cells are also implicated as critical determinants of disease and are similarly dependent on BCR-generated responses.

In preferred embodiments, benzodiazepines are selected because they are amenable to combinatorial synthesis (See, B. A. Bunin et al., Proc. Natl. Acad. Sci. U.S.A., 91:4708-4712 [1994]), and under certain conditions, some benzodiazepines influence cell survival (A. Beurdeley-Thomas et al., J. Neurooncol., 46:45-56 [2000]). Also, because benzodiazepines do not damage DNA or interfere with nucleotide metabolism, cytotoxic benzodiazepines would likely possess unique modes of action.

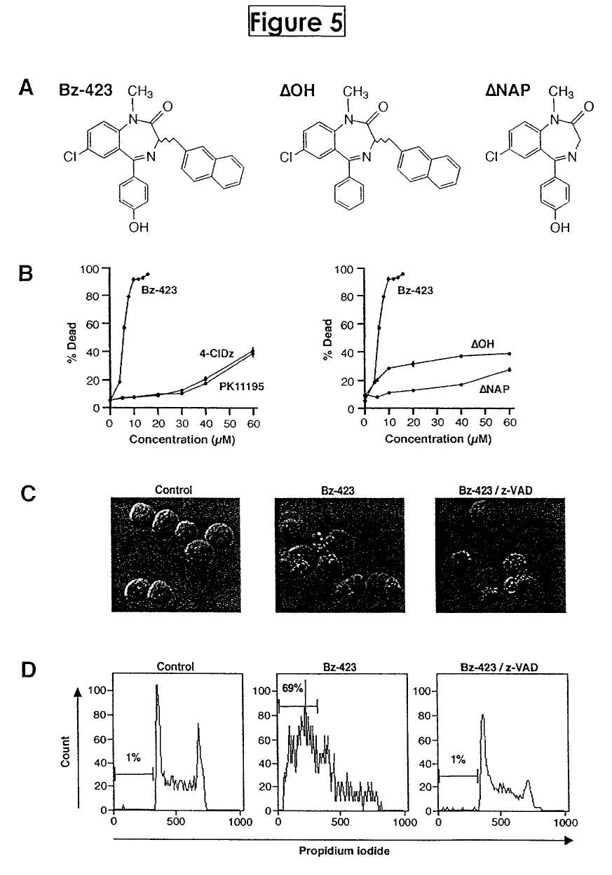

As described above, some preferred embodiments of the present invention identified Bz-423 (FIG. 5A) as a potent lead compound. Unlike benzodiazepines with anxiolytic properties, Bz-423 does not bind to the central benzodiazepine receptor. Certain embodiments of the present invention show that incubation of transformed Ramos B cells with Bz-423 rapidly generates O₂⁻ and this reactive oxygen species (ROS) functions as an upstream signal to commence an apoptotic death process. Although an understanding of the mechanism is not necessary to practice the present invention and the present invention is not so limited, it is contemplated that the O₂⁻ response results from the interaction between Bz-423 and a target within mitochondria, possibly in the mitochondrial respiratory chain (MRC). Because Ramos cells model aspects of germinal center (GC) B cell physiology (C. T. Gregory et al., J. Immunol., 139:313-318 [1987]), the present invention contemplates that Bz-423 has activity against GCs in vivo. In some embodiments, the activity of Bz-423 is shown using the (NZB×NZW)F1 (NZB/W) model of lupus where aberrant survival and expansion of GC B cells drives disease (See, J. P. Portanova et al., Mol. Immunol., 32:117-135 [1987]; and M. J. Shlomchik et al., Nat. Rev. Immunol., 1: 147-153 [2001]). In preferred embodiments, Bz-423 specifically controls GC hyperplasia and the subsequent development of glomerulonephritis in the NZB×NZW)F1 (NZB/W) mice model. Accordingly, the present invention contemplates a novel role for O₂⁻ in B cell apoptosis and identifies Bz-423 as a novel lead compound for the development of selective cytotoxic molecules to manage SLE and related disorders.

High levels of ROS that accompany the late stages of apoptosis damage macromolecules, whereas physiologic concentrations regulate a range of intracellular signaling pathways (T. Finkel, J. Leukoc. Biol., 65:337-340 [1999]). Some apoptotic stimuli (e.g., ceramide [See, C. Garcia-Ruiz et al., J. Biol. Chem., 272:11369-11377 (1997)]) dexamethasone (See, J. F. Torres-Roca et al., J. Immunol., 165:4822-4830 [2000]), or TNFα. (See, H. Albrecht et al., FEBS Lett., 351:45-48 [1994]), induce ROS early in their death responses as a means to initiate downstream effector mechanisms such as caspase activation. Similarly, the present invention shows that the early O₂⁻ response induced by interaction of Bz-423 with mitochondria signals an apoptotic program in B lymphocytes. Several B cell-specific signaling pathways respond to ROS and, when so engaged, initiate apoptosis. For example, activation of Bruton's tyrosine kinase by ROS (See, S. Qin et al., Proc. Natl. Acad. Sci. U.S.A., 97:7118-7123 [2000]) results in phosphorylation of phospholipase Cg (See, M. Takata and T. Kurosaki, J. Exp. Med., 184:31-40 [1996]), which can lead to Ca²⁺-dependent apoptosis. This and related ROS-dependent processes may contribute to the susceptibility of B cells to Bz-423.

The present invention provides a number of small molecules that increase intracellular O₂⁻ in a variety of ways, including, but not limited to, release of O₂⁻ from oxygenases, single electron reductions, inhibition of oxido-reductases, and disruption of MRC activity. (See, A. G. Siraki et al., Free Radic. Biol. Med., 32:2-10 [2002]).

Traditional chemotherapeutics compromise mitochondrial function indirectly. However, the some embodiments, of the present invention contemplate directly targeting the MRC or MPT pore. The critical elements of the Bz-423 response in vivo, O₂⁻ production and decreased B cell survival, are observed in NZB/W mice after dosing with Bz-423. In the NZB/W model of lupus, pathogenic autoantibodies are the products of activated, class-switched B cells that emerge from GCs (J. P. Portanova et al., Mol. Immunol., 32:117-135 [1987]; M. J. Shlomchik et al., Nat. Rev. Immunol., 1:147-153 [2001; and Y. Munakata et al., Eur. J. Immunol., 28:1435-1444 [1998]). In one preferred embodiment, after dosing for 12 wk, Bz-423 dramatically reduced the number and size of GCs and increased apoptosis in remaining GCs. Moreover, no significant decreases in other splenic lymphocyte populations, evidence of lymphopenia, or changes in cytokines was detected (unpublished data). Thus, the present invention contemplates that this B cell population is an important target for SLE intervention. (See e.g., M. J. Shlomchik et al., infra.).

The survival and apoptotic threshold GC B cells in normal mice is tightly regulated by c-FLIP and signaling through the BCR, CD40, CD80, and Fas (M. V. Eijk et al., Trends Immunol., 22:677-682 [2001]; and V. K. Tsiagbe et al., Crit. Rev. Immunol., 16:381-421 [1996]). Although an understanding of the mechanism is not necessary to practice the present invention and the present invention is not so limited, it is contemplated that the longevity of NZB/W GC B cells is tied to increased expression of co-stimulatory molecules like CD81, down regulation of inhibitory receptors such as FcgRIIB1 (See e.g., Y. Jiang et al., Int. Immunol., 11:1685-1691 [1999]), and hypo-responsiveness to persistent BCR stimulation (e.g., defective activation induced B cell death) (See e.g., Y. Kozono et al., J. Immunol., 156:4498-4503 [1999]). In this context, the present invention also notes that BCR stimulation also sensitizes B cells to cytotoxic agents. (See e.g., C. E. Lin et al., Exp. Cell. Res., 244:1-13 [1998]). Hence, in some embodiments it is contemplated that receptor ligation contributes to the selectivity of Bz-423. For example, studies show that anti-IgM sensitizes B cells to Bz-423. While the molecular basis for BCR sensitization is not fully understood, it is known that cross-linking BCRs can itself elevate intracellular ROS (See e.g., W. Fang et al., J. Immunol., 155:66-75 [1995]) and that lymphocytes from lupus patients have decreased stores of reduced glutathione and increased ROS relative to normal cells (See e.g., P. Gergely Jr. et al., Arthritis Rheum., 46:175-190 [2002]). Thus, the vulnerability of NZB/W GC B cells may, in part, result from an additive effect of Bz-423-induced O₂⁻ and endogenously generated ROS, such that radicals from all sources combine to overwhelm the limited reducing potential of these lymphocytes and trigger apoptosis.

Bz-423 is a pro-apoptotic molecule that engages the cell-death machinery in an O₂⁻ dependent manner. The present invention provides a new structure-function relationship for benzodiazepines and points to a new molecular target and pharmacological mechanism valuable for the management of SLE.

The present invention further contemplates that Bz-423 kills Ramos B cells in a dose-dependent fashion (FIG. 5B). The activity of Bz-423 was compared to ligands of the peripheral benzodiazepine receptor (PBR), an 18 KDa transmembrane protein located in the mitochondrial membrane, because some ligands of the PBR are thought to modulate death signals from mitochondria. (See e.g., A. Beurdeley-Thomas et al., J. Neurooncol., 46:45-56 [2000]). Both 1-(2-chlorophenyl)-N-methyl-N-(1-methylpropyl)-3-isoquinolinecarboxamide (PK1 1195) and 4′-chlorodiazepam bind tightly to the PBR (KD=0.3 and 30 nM, respectively; but are only cytotoxic at >10-times the ED50 of Bz-423. Competition assays for the PBR demonstrate that a 1000-fold excess of Bz-423 is necessary to reduce [3H]-PK11195 binding by 50%, and pre-incubation of cells with excess PK11195 (>20 μM) does not block the activity of Bz-423. These data indicate that cell killing by Bz-423 does not result from binding to the PBR. The activity of Bz-423 depends on its specific structure, since deleting either the napthyl (DNAP) or the phenolic hydroxyl (DOH) groups, elements that distinguish Bz-423 from diazepam, dramatically reduces cytotoxic activity (FIG. 5B). Bz-423-induced cytotoxicity is characterized by cell shrinkage, nuclear condensation, cytoplasmic vacuolization, membrane blebbing, and DNA fragmentation (hypodiploid DNA; FIGS. 5C and 5D), consistent with apoptosis. To probe the role of caspases in Bz-423 killing, cells were pre-incubated with z-VAD, an irreversible caspase inhibitor. z-VAD completely prevents Bz-423-mediated apoptosis as measured by DNA fragmentation. Less than 5% of cells treated with z-VAD and Bz-423 have hypodiploid DNA, compared to 69% of cells treated with Bz-423 alone (FIG. 5D). However, inhibiting caspase activity does not protect against cell death; cellular morphology demonstrates that Bz-423 still kills >80% of cells in cultures containing z-VAD (See, FIG. 5C). Similar results have been reported for other apoptotic stimuli, where blocking caspase activity prevents DNA fragmentation but does not inhibit overall cell death (A. S. Belzacq et al., Cancer Res., 61:1260-1264 [2001]).

In yet other embodiments, in experiments with isolated mitochondria under conditions supporting state 3 respiration demonstrate an O₂— response analogous to that in whole cells. Mitochondria do not respond to Bz-423 in state 4 where energy is supplied in the absence of ADP. Although an understanding of the mechanism is not necessary to practice the present invention and the present invention is not so limited, it is contemplated that, in transitioning from state 3 to 4, the proton motive force becomes sufficiently high that intermediates competent in one electron reduction reactions (e.g., ubiquinol) have extended half-lives, and these conditions favor reduction of O₂ to O₂— at complex III. (See, V. P. Skulachev, Mol. Aspects Med., 20:139-184 [1999]). Hence, in some embodiments, it is possible that Bz-423 generates O₂— by inducing a state 3 to 4 conversion. Oligomycin, a macrolide natural product that binds to complex V, induces a state 3 to 4 transition and generates ° 2-like Bz-423. These similarities indicate that complex V is also the molecular target for Bz-423. Complex V is a large multi-protein assembly and can be inhibited by small molecules in a number of different ways. However, it is contemplated in some embodiments that the similarities to oligomycin suggests that Bz-423 may inhibit the ATPase activity of complex V by binding to an element of complex V that includes the oligomycin sensitivity conferring protein, which is in the F1 domain.

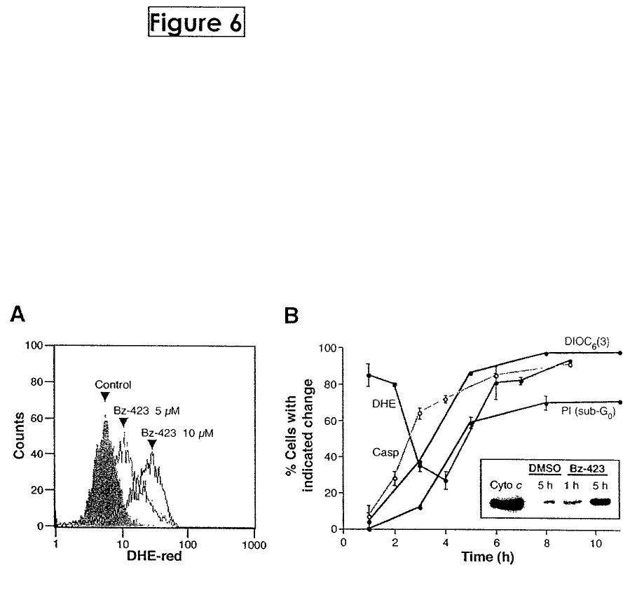

In additional embodiments, to further characterize the death mechanism engaged by Bz-423, intracellular ROS, ΔΨₘ, cytochrome c release, caspase activation, and DNA fragmentation were measured over time. In preferred embodiments, the present invention used endpoints as previously implicated in B cell apoptosis. (See e.g., T. Doi et al., Int. Immunol., 11:933-941 [1999]). The first event detected after exposure to Bz-423 is an increase in the fraction of cells that stain with DHE, a redox-sensitive agent that reacts specifically with O₂⁻ (a dose-dependent increase in the mean fluorescence intensity was also observed; FIG. 6A). Levels of O₂⁻ diminish after an early maximum at 1 h and then increase again after 4 h of continued treatment (FIG. 6B). This bimodal pattern points to a cellular mechanism limiting O₂⁻ and suggests that the “early” and “late” O₂⁻ maxima result from different processes. Collapse of ΔΨₘ was detected using DiOC₆(3), a mitochondria-selective potentiometric probe (N. Zamzami et al., J. Exp. Med. 182:367-377 [1995]). The gradient change begins after the early O₂⁻ response and is observed in >90% of cells by 5 h. Cytochrome c release from mitochondria, a key step enabling apoptosome formation and caspase activation (P. Li et al., Cell, 91:479-489 [1997]), was studied by immunoblotting cytosolic fractions. Levels of cytosolic cytochrome c above amounts in cells treated with vehicle are detected by 5 h (inset, FIG. 6B). This release is coincident with the disruption of ΔΨₘ, and together, these results are consistent with opening of the mitochondrial permeability transition (MPT) pore. Indeed, the late increase in O₂⁻ closely tracks with the ΔΨₘ collapse and the release of cytochrome c, suggesting that the secondary rise in O₂⁻ results from these processes. (See e.g., C. M. Luetjens et al., J. Neurosci., 20:5715-5723 [2000]). Caspase activation, measured by processing of the pan-caspase sensitive fluorescent substrate FAM-VAD-fluoromethylketone, tracks the gradient changes, whereas the appearance of hypodiploid DNA is slightly delayed with respect to caspase activation (FIG. 6B). Although an understanding of the mechanism is not necessary to practice the present invention and the present invention is not so limited, it is contemplated that Bz-423 induces a mitochondrial dependent apoptotic pathway.

In some embodiments of the present invention, Bz-423 is contemplated to directly target mitochondria. Since early O₂⁻ precedes caspase activation, collapse of ΔΨₘ, and DNA fragmentation, it is possible that this ROS has a regulatory role. In non-phagocytic cells, redox enzymes, along with the MRC, are the primary sources of ROS. (See e.g., T. Finkel, J. Leukoc. Biol., 65:337-340 [1999]). Therefore, in some embodiments, the present invention assayed inhibitors of these systems for their ability to regulate Bz-423-induced O₂⁻ (at 1 h) to determine the basis for this response (Table 2). Of these reagents, only NaN₃, which acts primarily on complex IV of the MRC (E. Fosslien, Ann. Clin. Lab. Sci., 31:25-67 [2001]), and micromolar amounts of FK-506, which block the formation of O₂⁻ at complex III (R. Zini et al., Life Sci., 63:357-368 [1998]), modulate Bz-423. Other agents that inhibit components of the MRC (e.g., rotenone, myxothiazol, thenoyltrifluoroacetone, antimycin A, stigmatellin, and oligomycin), along with the flavoenzyme inhibitor diphenyleneiodonium, were less informative as they independently generate significant ROS in Ramos cells. Collectively, these findings suggested that mitochondria are the source of Bz-423-induced O₂⁻ and that a component of the MRC may be involved in the response. Although the inhibition by FK506 could result from binding to either calcineurin or FK506-binding proteins, natural products that bind tightly to these proteins (e.g., rapamycin and cyclosporin A, respectively) do not diminish the Bz-423 O₂⁻ response (Table 2). Table 2 shows the effect of ROS inhibitors on the activity of Bz-423.

TABLE 2

| | | | % DHE | |
|---|---|---|---|---|
| | | % DHE | positive: | |
| | | positive: | Bz-423 | |
| | | Inhibitor | plus | Relative |
| Target | Inhibitor | alone | inhibitor | response^{A} |
| | DMSO control | 7 | 72 | 100 (±13) |
| MRC | NaN₃ | 0 | 0 | 1 (±1) |
| | (1 mM) | | | |
| MRC/ | FK506 | 1 | 10 | 14 (±10) |
| Calcineurin | (1 μM) | | | |
| FK506-binding | Rapamycin | 1 | 60 | 83 (±2) |
| proteins | (1 μM) | | | |
| Calcineurin | Cyclosporin A | 2 | 60 | 83 (±2) |
| | (0.5 μM) | | | |
| Cytochrome | Benzyli- | 14 | 90 | 126 (±7) |
| P450s | midazole | | | |
| | (100 μM) | | | |
| Cytochrome | SKF525A | 12 | 92 | 128 (±6) |
| P450s | (5 μM) | | | |
| Cytochrome | Cimetidine | 6 | 83 | 116 (±7) |
| P450 | (100 μM) | | | |
| (CYP231) | | | | |
| Cyclooxygenase | Indomethacin | 4 | 69 | 97 (±4) |
| | (100 μM) | | | |
| Monoaminoxidase | Phenelzine | 5 | 78 | 109 (±4) |
| | (10 μM) | | | |
| Xanthine | Allopurinol | 6 | 79 | 111 (±7) |
| Oxidase | (100 μM) | | | |

^{A}Data are normalized to the response of Bz-423 alone.

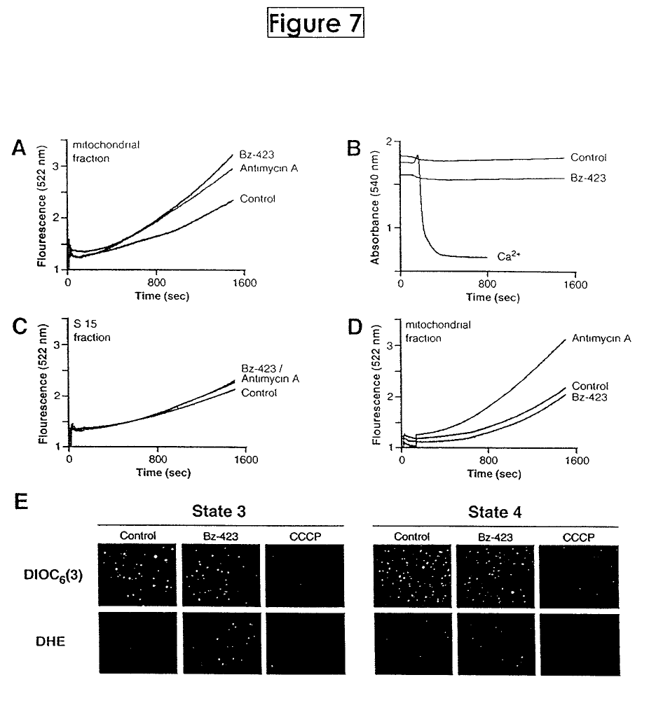

Although an understanding of the mechanism is not necessary to practice the present invention and the present invention is not so limited, it is contemplated that the way in which Bz-423 produces O₂⁻ involves binding to a protein within mitochondria or a target in another compartment that signals mitochondria to generate O₂⁻. To distinguish between these alternatives, isolated rat liver mitochondria were assayed for ROS production in the presence and absence of Bz-423. ROS were detected by monitoring the oxidation of DCFH-DA to DCF as previously described in Esposti. (See, M. D. Esposti, Methods Cell. Biol., 65:75-96 [2001]). In this assay, the rate of DCF production increases after a lag period of ca. 15 min, during which time endogenous reducing equivalents within mitochondria are consumed and acetate moieties on the probe are hydrolyzed to yield DCFH, which is the redox-active species. Under aerobic conditions supporting state 3 respiration (succinate plus ADP), both antimycin A, which generates O₂⁻ by inhibiting ubiquinol-cytochrome c reductase within complex III, and Bz-423 increase the rate of ROS production nearly two-fold relative to solvent control (See, Esposti, infra.) (FIG. 7A). Mitochondrial swelling is not observed demonstrating that Bz-423 does not directly target the MPT pore. (A. S. Belzacq et al., Cancer Res., 61:1260-1264 [2001]). (FIG. 7B). Neither Bz-423 nor antimycin A generate substantial ROS in the subcellular S115 fraction (cytosol and microsomes; FIG. 7C), and Bz-423 does not stimulate ROS if mitochondria are in state 4 (succinate plus oligomycin), even though antimycin A is active under these conditions (FIG. 7D). Together, these experiments demonstrate that mitochondria contain a molecular target for Bz-423, and state 3 respiration is required for the O₂⁻ response.

To relate data obtained with mitochondria to observations in whole cells, isolated mitochondria were also probed with DHE and DIOC₆(3). In both state 3 and 4, mitochondria fluoresce brightly with DIOC₆(3), indicating that ΔΨₘ is intact (FIG. 7E). Addition of Bz-423 does not alter the DIOC₆(3) signal over the course of measurement, indicating that ΔΨₘ has not collapsed. In contrast, the protonophore CCCP disrupts ΔΨₘ, which abolishes the green fluorescence. Oxidation of DHE by endogenous O₂⁻ produces red fluorescence within mitochondria. Bz-423 does not induce O₂⁻ over control in state 4. However, DHE fluorescence is markedly increased after incubating mitochondria with Bz-423 in buffer favoring state 3 respiration and reflects elevated levels of O₂⁻. Collectively, these experiments confirm the results of the DCF assay, demonstrate an ROS response in mitochondria consistent with that seen in whole cells, and provide evidence to support the hypothesis that the early O₂⁻ does not result from collapse of the mitochondrial transmembrane gradient (See, FIG. 6B).

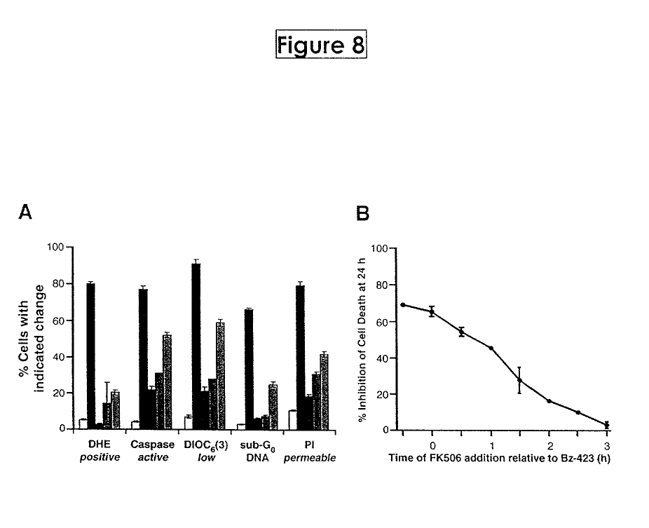

In some embodiments, the present invention contemplates that Bz-423-induced apoptosis depends on the early O₂⁻ response. To determine if O₂⁻ is needed for Bz-423-induced apoptosis, some embodiments of the present invention probed the dependency of the apoptotic process on ROS. Pre-treating cells with FK506, which prevents formation of Bz-423-induced O₂⁻, significantly inhibits caspase activation, mitochondrial depolarization, DNA fragmentation, and cell death (FIG. 8A). Moreover, after cells are incubated with Bz-423 for ca. 1 h, which is the point at which the early O₂⁻ response is maximal (See, FIG. 6B), addition of FK506 provides significantly less protection against cell death (FIG. 8B). Pre-incubating cells with vitamin E, an antioxidant that scavenges ROS, or MnTBAP, an O₂⁻ dismutase mimetic, also attenuates each of these endpoints (FIG. 8A). MnTBAP is somewhat less effective at influencing the downstream effectors engaged by Bz-423 because dismutation of O₂⁻ yields H₂O₂, which also triggers the same effectors (T. Ohse et al., J. Inor. Biochem., 85:201-208 [2001]) (FIG. 8A). These data demonstrate that cell killing by Bz-423 depends on O₂⁻.

Still further embodiments of the present invention provide methods of using Bz-423 that induce ROS and kill primary B lymphocytes in vivo. In the NZB/W model of lupus, splenic GC B cells are hyperactivated and pathologically expanded. Somatic hypernutation within this compartment produces anti-DNA and other autoantibodies, a subset of which are pathogenic and contribute to the development of glomerulonephritis (See e.g., J. P. Portanova et al., Mol. Immunol., 32:117-135 [1987]; Y. Munakata et al., Eur. J. Immunol., 28:1435-1444 [1998]; and A. N. Theofilopoulos and F. J. Dixon, Adv. Immunol., 37:269-390 [1985]). Because Ramos cell behavior parallels many of the responses of GC B cells (See, C. T. Gregory et al., J. Immunol., 139:313-318 [1987]), preferred embodiments of the present invention contemplate that Bz-423 has activity in NZB/W mice that could be therapeutically useful. To characterize the effect of Bz-423 on NZB/W lymphocytes, mice were given a single dose of either Bz-423 (60 mg/kg; n=4) or vehicle (n=4) and sacrificed after 2 h. This dose provides peak serum levels of ca. 5 μM 1 h post-injection, which is near the EC₅₀ in vitro (See, FIG. 5B). Bz-423 induces robust ROS production in the spleen compared to control. Based on these findings, Bz-423 was administered (60 mg/kg/d) for 7 d to determine whether it was lymphotoxic in the murine model. A short time frame was chosen so that cellular recruitment or proliferation would not significantly alter splenocyte populations. Flow cytometric analysis after sacrifice shows that lymphocyte viability in the Bz-423-treated animals is decreased (81±1 vs. 85±1; P=0.006) and B cell apoptosis is increased versus controls (32±2 vs. 25±2; P=0.02). The decrease in viability is relatively small because early apoptotic cells are rapidly cleared by the reticuloendothelial system in vivo (N. Zamzami et al., J. Exp. Med. 182:367-377 [1995]). An increase in the frequency of T cell apoptosis was not observed (12±4 for both control and treatment; P=0.3).

Bz-423 reduces lymphoproliferative and autoimmune disease in NZB/W mice. In certain embodiments, additional studies were conducted to determine if Bz-423 alters the progression of disease in NZB/W mice. The endpoints of these studies were measures of GC B cell hyperplasia, glomerulonephritis, and autoantibody titers, respectively. In one protocol, female NZB/W mice were given Bz-423 (60 mg/kg/every other day; n=25) or vehicle (aqueous DMSO; n=20) from 6.5 to 9.5 months of age. This time frame begins after the histological onset of disease and continues to when severe nephritis is usually observed. (See e.g., A. N. Theofilopoulos and F. J. Dixon, Adv. Immunol., 37:269-390 [1985]). Disease-related GC hyperplasia in NZB/W mice leads to an expansion of the white pulp that distorts normal splenic architecture. Analyzing spleen sections from all treatment and control animals revealed a reduction in the white pulp in mice receiving Bz-423 (1-2+ vs. 3-4+; P=0.018). Mice dosed with Bz-423 have 40% fewer GCs relative to controls (10±2 vs. 17±1 per 10 mm2, P=0.009), and the GCs in treated mice are 40% smaller than in the controls (20±2 vs. 35+5×103 mm2, P=0.013). Furthermore, spleens from Bz-423-treated animals have more TUNEL-positive B cells within GCs (ca. 3+vs. 1+, P=0.038) than controls, and such differences in TUNEL staining are not observed in other areas of the spleen. These observations suggest that the decrease in GCs may result from increased apoptosis within this compartment. In some embodiments, the changes in splenocyte populations were also investigated by flow cytometry. At the end of the study, a specific effect on B cells was observed (Bz-423: 59±3, control: 67±2%; P=0.03) with no measurable effect on T cells (Bz-423: 16±2, control: 17±2%; P=0.5), which is consistent with the histochemical findings.

Based on histology, 60% of controls (12 of 20) had severe nephritis (>2+), while only 16% of Bz-423-treated mice (4 of 25) had disease (c2: P=0.003). Disease in the control animals is characterized by diffuse, proliferative glomerulonephritis with expansion of all cellular elements and occasional wire-loop formation, consistent with an average histopathological score of 3+. In contrast, Bz-423 mice have milder changes (1+, P=0.002) and less glomerular deposition of IgG (1+for Bz-423 vs. 3+in controls, P=0.002). At the end of the study, 83% of controls had abnormally high BUN (≧30 mg/dL), compared to 27% of the treatment group (c2: P=0.001). A similar trend was observed with proteinuria: 49% of controls had significant proteinuria (>100 mg/dL) compared to 18% of Bz-423-treated mice (c2: P=0.1). Collectively, these data show that mice administered Bz-423 had less disease than controls.

Treating NZB/W mice with lymphotoxic drugs like azathioprine and methylprednisolone can reduce nephritis without significantly altering total serum anti-DNA levels. (See e.g., M. C. Gelfand and A. D. Steinberg, Arthritis Rheum., 15:247-255 [1972]). Similarly, anti-DNA and IgG titers were measured after terminating the study and significant differences between the groups were not observed (e.g., Bz-423 anti-dsDNA: 789±145, control: 733±198 U/mL; P=0.5; Bz-423 IgG: 6.7±0.8, control: 5.5±1.0 mg/mL; P=0.3). Although autoantibodies are produced by several B cell subtypes, Bz-423 reduces GCs, the site of pathogenic autoantibody development.

III. Synthesis of Exemplary Benzodiazepine Derivatives

The compounds of the present invention are benzodiazepine compounds. In some aspects, the benzodiazepine compounds have the following structure:

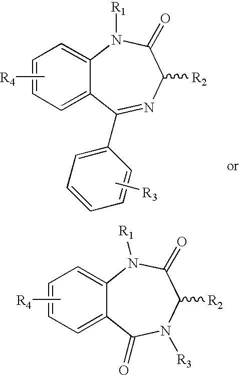

or its enantiomer, wherein, R₁ is aliphatic or aryl; R₂ is aliphatic, aryl, —NH₂, —NHC(═O)—R₅; or a moiety that participates in hydrogen bonding, wherein R₅ is aryl, heterocyclic, —R₆—NH—C(═O)—R₇ or —R₆—C(═O)—NH—R₇, wherein R₆ is an aliphatic linker of 1-6 carbons and R₇ is aliphatic, aryl, or heterocyclic, each of R₃ and R₄ is independently a hydroxy, alkoxy, halo, amino, lower-alkyl-substituted-amino, acetylamino, hydroxyamino, an aliphatic group having 1-8 carbons and 1-20 hydrogens, aryl, or heterocyclic; or a pharmaceutically acceptable salt, prodrug or derivative thereof.

In the above structures, R₁ is a hydrocarbyl group of 1-20 carbons and 1-20 hydrogens. Preferably, R₁ has 1-15 carbons, and more preferably, has 1-12 carbons. Preferably, R₁ has 1-12 hydrogens, and more preferably, 1-10 hydrogens. Thus R₁ can be an aliphatic group or an aryl group.

The term “aliphatic” represents the groups commonly known as alkyl, alkenyl, alkynyl, alicyclic. The term “aryl” as used herein represents a single aromatic ring such as a phenyl ring, or two or more aromatic rings that are connected to each other (e.g., bisphenyl) or fused together (e.g., naphthalene or anthracene). The aryl group can be optionally substituted with a lower aliphatic group (e.g., C₁-C₄ alkyl, alkenyl, alkynyl, or C₃-C₆ alicyclic). Additionally, the aliphatic and aryl groups can be further substituted by one or more functional groups such as —NH₂, —NHCOCH₃, —OH, lower alkoxy (C₁-C₄), halo (—F, —Cl, —Br, or —I). It is preferable that R₁ is primarily a nonpolar moiety.

In the above structures, R₂ can be aliphatic, aryl, —NH₂, —NHC(═O)—R₅, or a moiety that participates in hydrogen bonding, wherein R₅, is aryl, heterocyclic, R₆—NH—C(═O)—R₇ or —R₆—C(═O)—NH—R₇, wherein R₆ is an aliphatic linker of 1-6 carbons and R₇ is an aliphatic, aryl, or heterocyclic. The terms “aliphatic” and “aryl” are as defined above.

The term “a moiety that participates in hydrogen bonding” as used herein represents a group that can accept or donate a proton to form a hydrogen bond thereby.

Some specific non-limiting examples of moieties that participate in hydrogen bonding include a fluoro, oxygen-containing and nitrogen-containing groups that are well-known in the art. Some examples of oxygen-containing groups that participate in hydrogen bonding include: hydroxy, lower alkoxy, lower carbonyl, lower carboxyl, lower ethers and phenolic groups. The qualifier “lower” as used herein refers to lower aliphatic groups (C₁-C₄) to which the respective oxygen-containing functional group is attached.

Thus, for example, the term “lower carbonyl” refers to inter alia, formaldehyde, acetaldehyde.

Some nonlimiting examples of nitrogen-containing groups that participate in hydrogen bond formation include amino and amido groups. Additionally, groups containing both an oxygen and a nitrogen atom can also participate in hydrogen bond formation. Examples of such groups include nitro, N-hydroxy and nitrous groups.

It is also possible that the hydrogen-bond acceptor in the present invention can be the π electrons of an aromatic ring. However, the hydrogen bond participants of this invention do not include those groups containing metal atoms such as boron. Further the hydrogen bonds formed within the scope of practicing this invention do not include those formed between two hydrogens, known as “dihydrogen bonds.” (See, R. H. Crabtree, Science, 282:2000-2001 [1998], for further description of such dihydrogen bonds).

The term “heterocyclic” represents, for example, a 3-6 membered aromatic or nonaromatic ring containing one or more heteroatoms. The heteroatoms can be the same or different from each other. Preferably, at least one of the heteroatom's is nitrogen. Other heteroatoms that can be present on the heterocyclic ring include oxygen and sulfur.

Aromatic and nonaromatic heterocyclic rings are well-known in the art. Some nonlimiting examples of aromatic heterocyclic rings include pyridine, pyrimidine, indole, purine, quinoline and isoquinoline. Nonlimiting examples of nonaromatic heterocyclic compounds include piperidine, piperazine, morpholine, pyrrolidine and pyrazolidine. Examples of oxygen containing heterocyclic rings include, but not limited to furan, oxirane, 2H-pyran, 4H-pyran, 2H-chromene, and benzofuran. Examples of sulfur-containing heterocyclic rings include, but are not limited to, thiophene, benzothiophene, and parathiazine.

Examples of nitrogen containing rings include, but not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine.

Examples of heterocyclic rings containing two different heteroatoms include, but are not limited to, phenothiazine, morpholine, parathiazine, oxazine, oxazole, thiazine, and thiazole.

The heterocyclic ring is optionally further substituted with one or more groups selected from aliphatic, nitro, acetyl (i.e., —C(═O)—CH₃), or aryl groups.

Each of R₃ and R₄ can be independently a hydroxy, alkoxy, halo, amino, or substituted amino (such as lower-alkyl-substituted-amino, or acetylamino or hydroxyamino), or an aliphatic group having 1-8 carbons and 1-20 hydrogens. When each of R₃ and R₄ is an aliphatic group, it can be further substituted with one or more functional groups such as a hydroxy, alkoxy, halo, amino or substituted amino groups as described above. The terms “aliphatic” is defined above. Alternatively, each of R₃ and R₄ can be hydrogen.

It is well-known that many 1,4-benzodiazepines exist as optical isomers due to the chirality introduced into the heterocyclic ring at tile C₃ position. The optical isomers are sometimes described as L- or D-isomers in the literature. Alternatively, the isomers are also referred to as R— and S— enantiomorphs. For the sake of simplicity, these isomers are referred to as enantiomorphs or enantiomers. The 1,4-benzodiazepine compounds described herein include their enantiomeric forms as well as racemic mixtures. Thus, the usage “benzodiazepine or its enantiomers” herein refers to the benzodiazepine as described or depicted, including all its enantiomorphs as well as their racemic mixture.

From the above description, it is apparent that many specific examples are represented by the generic formulas presented above. Thus, in one example, R₁ is aliphatic, R₂ is aliphatic, whereas in another example, R₁ is aryl and R₂ is a moiety that participates in hydrogen bond formation. Alternatively, R₁ can be aliphatic, and R₂ can be an —NHC(═O)—R₅, or a moiety that participates in hydrogen bonding, wherein R₅ is aryl, heterocyclic, —R₆—NH—C(═O)—R₇ or —R₆—C(═O)—NH—R₇, wherein R₆ is an aliphatic linker of 1-6 carbons and R₇ is an aliphatic, aryl, or heterocyclic. A wide variety of sub combinations arising from selecting a particular group at each substituent position are possible and all such combinations are within the scope of this invention.

Further, it should be understood that the numerical ranges given throughout this disclosure should be construed as a flexible range that contemplates any possible subrange within that range. For example, the description of a group having the range of 1-10 carbons would also contemplate a group possessing a subrange of, for example, 1-3, 1-5, 1-8, or 2-3, 2-5, 2-8, 3-4, 3-5, 3-7, 3-9, 3-10, etc., carbons. Thus, the range 1-10 should be understood to represent the outer boundaries of the range within which many possible subranges are clearly contemplated. Additional examples contemplating ranges in other contexts can be found throughout this disclosure wherein such ranges include analogous subranges within.

Some specific examples of the benzodiazepine compounds of this invention include:

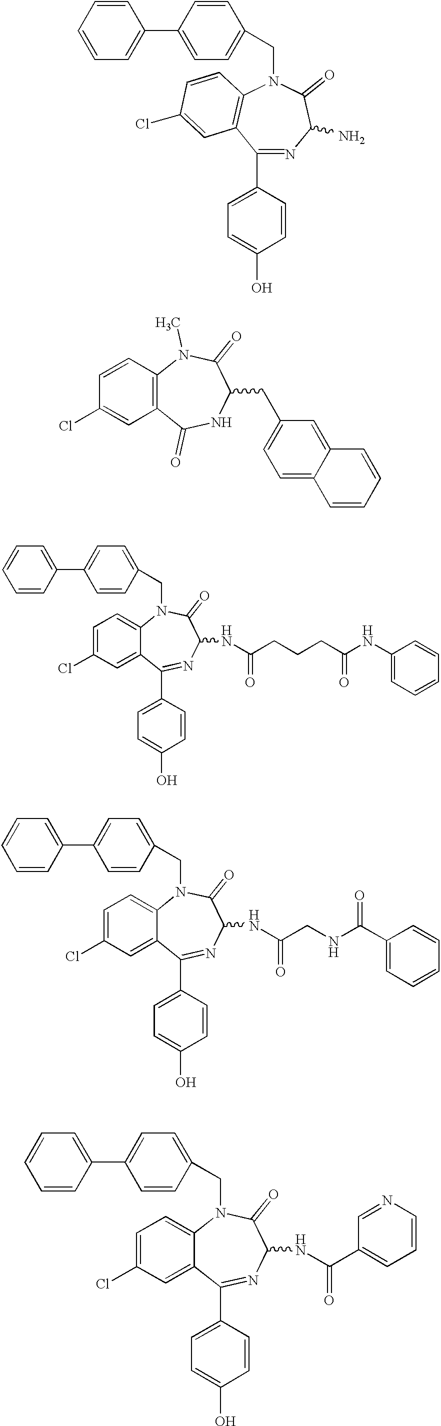

In summary, a large number of benzodiazepine compounds are presented herein. Any one or more of these benzodiazepine compounds can be used to treat a variety of dysregulatory disorders related to cellular death as described elsewhere herein. The above-described benzodiazepines can also be used in drug screening assays and other diagnostic methods.

IV. Pharmaceutical Compositions, Formulations, and Exemplary Administration Routes and Dosing Considerations

Exemplary embodiments of various contemplated medicaments and pharmaceutical compositions are provided below.

A. Benzodiazepine Derivatives as Compounds for Preparing Medicaments

The benzodiazepine compounds of the present invention useful in the preparation of medicaments to treat a variety of conditions associated with dysregulation of cell death, aberrant cell growth and hyperproliferation.

In addition, the compounds are also useful for preparing medicaments for treating other disorders wherein the effectiveness of the benzodiazepines are known or predicted. Such disorders may include, but are not limited to, neurological (e.g., epilepsy) or neuromuscular disorders. The methods and techniques for preparing medicaments of a compound are well-known in the art. Exemplary pharmaceutical formulations and routes of delivery are described below.

One of skill in the art will appreciate that any one or more of the compounds described herein, including the many specific embodiments, are prepared by applying standard pharmaceutical manufacturing procedures. Such medicaments can be delivered to the subject by using delivery methods that are well-known in the pharmaceutical arts.

B. Exemplary Pharmaceutical Compositions and Formulation

In some embodiments of the present invention, the compositions are administered alone, while in some other embodiments, the compositions are preferably present in a pharmaceutical formulation comprising at least one active ingredient/agent (e.g., benzodiazepine derivative), as defined above, together with a solid support or alternatively, together with one or more pharmaceutically acceptable carriers and optionally other therapeutic agents. Each carrier must be “acceptable” in the sense that it is compatible with the other ingredients of the formulation and not injurious to the subject.

Contemplated formulations include those suitable oral, rectal, nasal, topical (including transdermal, buccal and sublingual), vaginal, parenteral (including subcutaneous, intramuscular, intravenous and intradermal) and pulmonary administration. In some embodiments, formulations are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present invention suitable for oral administration may be presented as discrete units such as capsules, cachets or tablets, wherein each preferably contains a predetermined amount of the active ingredient; as a powder or granules; as a solution or suspension in an aqueous or non-aqueous liquid; or as an oil-in-water liquid emulsion or a water-in-oil liquid emulsion. In other embodiments, the active ingredient is presented as a bolus, electuary, or paste, etc.

In some embodiments, tablets comprise at least one active ingredient and optionally one or more accessory agents/carriers are made by compressing or molding the respective agents. In preferred embodiments, compressed tablets are prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose)surface-active or dispersing agent. Molded tablets are made by molding in a suitable machine a mixture of the powdered compound (e.g., active ingredient) moistened with an inert liquid diluent. Tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention are optionally formulated as ointments, creams, suspensions, lotions, powders, solutions, pastes, gels, sprays, aerosols or oils. In alternatively embodiments, topical formulations comprise patches or dressings such as a bandage or adhesive plasters impregnated with active ingredient(s), and optionally one or more excipients or diluents. In preferred embodiments, the topical formulations include a compound(s) that enhances absorption or penetration of the active agent(s) through the skin or other affected areas. Examples of such dermal penetration enhancers include dimethylsulfoxide (DMSO) and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this invention are constituted from known ingredients in an known manner. This phase typically comprises an lone emulsifier (otherwise known as an emulgent), it is also desirable in some embodiments for this phase to further comprises a mixture of at least one emulsifier with a fat or an oil or with both a fat and an oil.

Preferably, a hydrophilic emulsifier is included together with a lipophilic emulsifier so as to act as a stabilizer. It some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate.

Formulations suitable for vaginal administration may be presented as pessaries, creams, gels, pastes, foams or spray formulations containing in addition to the agent, such carriers as are known in the art to be appropriate.

Formulations suitable for nasal administration, wherein the carrier is a solid, include coarse powders having a particle size, for example, in the range of about 20 to about 500 microns which are administered in the manner in which snuff is taken, i.e., by rapid inhalation (e.g., forced) through the nasal passage from a container of the powder held close up to the nose. Other suitable formulations wherein the carrier is a liquid for administration include, but are not limited to, nasal sprays, drops, or aerosols by nebulizer, an include aqueous or oily solutions of the agents.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. In some embodiments, the formulations are presented/formulated in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of an agent.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoleic acid), extenders, and stabilizers, etc.

C. Exemplary Administration Routes and Dosing Considerations

Various delivery systems are known and can be used to administer a therapeutic agents (e.g., benzodiazepine derivatives) of the present invention, e.g., encapsulation in liposomes, microparticles, microcapsules, receptor-mediated endocytosis, and the like. Methods of delivery include, but are not limited to, intra-arterial, intramuscular, intravenous, intranasal, and oral routes. In specific embodiments, it may be desirable to administer the pharmaceutical compositions of the invention locally to the area in need of treatment; this may be achieved by, for example, and not by way of limitation, local infusion during surgery, injection, or by means of a catheter.

The agents identified herein as effective for their intended purpose can be administered to subjects or individuals susceptible to or at risk of developing pathological growth of target cells and condition correlated with this. When the agent is administered to a subject such as a mouse, a rat or a human patient, the agent can be added to a pharmaceutically acceptable carrier and systemically or topically administered to the subject. To determine patients that can be beneficially treated, a tissue sample is removed from the patient and the cells are assayed for sensitivity to the agent.

Therapeutic amounts are empirically determined and vary with the pathology being treated, the subject being treated and the efficacy and toxicity of the agent. When delivered to an animal, the method is useful to further confirm efficacy of the agent. One example of an animal model is MLR/MpJ-lpr/lpr (“MLR-lpr”) (available from Jackson Laboratories, Bal Harbor, Me.). MLR-lpr mice develop systemic autoimmune disease. Alternatively, other animal models can be developed by inducing tumor growth, for example, by subcutaneously inoculating nude mice with about 10⁵ to about 10⁹ hyperproliferative, cancer or target cells as defined herein. When the tumor is established, the compounds described herein are administered, for example, by subcutaneous injection around the tumor. Tumor measurements to determine reduction of tumor size are made in two dimensions using venier calipers twice a week. Other animal models may also be employed as appropriate. Such animal models for the above-described diseases and conditions are well-known in the art.

In some embodiments, in vivo administration is effected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

Suitable dosage formulations and methods of administering the agents are readily determined by those of skill in the art. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including, but not limited to, oral, rectal, nasal, topical (including, but not limited to, transdermal, aerosol, buccal and sublingual), vaginal, parental (including, but not limited to, subcutaneous, intramuscular, intravenous and intradermal) and pulmonary. It is also appreciated that the preferred route varies with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient.

Desirable blood levels of the agent may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

D. Exemplary Co-Administration Routes and Dosing Considerations

The present invention also includes methods involving co-administration of the compounds described herein with one or more additional active agents. Indeed, it is a further aspect of this invention to provide methods for enhancing prior art therapies and/or pharmaceutical compositions by co-administering a compound of this invention. In co-administration procedures, the agents may be administered concurrently or sequentially. In one embodiment, the compounds described herein are administered prior to the other active agent(s). The pharmaceutical formulations and modes of administration may be any of those described above. In addition, the two or more co-administered chemical agents, biological agents or radiation may each be administered using different modes or different formulations.

The agent or agents to be co-administered depends on the type of condition being treated. For example, when the condition being treated is cancer, the additional agent can be a chemotherapeutic agent or radiation. When the condition being treated is an autoimmune disorder, the additional agent can be an immunosuppressant or an anti-inflammatory agent. When the condition being treated is chronic inflammation, the additional agent can be an anti-inflammatory agent. The additional agents to be co-administered, such as anticancer, immunosuppressant, anti-inflammatory, and can be any of the well-known agents in the art, including, but not limited to, those that are currently in clinical use. The determination of appropriate type and dosage of radiation treatment is also within the skill in the art or can be determined with relative ease.

Treatment of the various conditions associated with abnormal apoptosis is generally limited by the following two major factors: (1) the development of drug resistance and (2) the toxicity of known therapeutic agents. In certain cancers, for example, resistance to chemicals and radiation therapy has been shown to be associated with inhibition of apoptosis. Some therapeutic agents have deleterious side effects, including non-specific lymphotoxicity, renal and bone marrow toxicity.

The methods described herein address both these problems. Drug resistance, where increasing dosages are required to achieve therapeutic benefit, is overcome by co-administering the compounds described herein with the known agent. The compounds described herein appear to sensitize target cells to known agents and, accordingly, less of these agents are needed to achieve a therapeutic benefit.

The sensitizing function of the claimed compounds also addresses the problems associated with toxic effects of known therapeutics. In instances where the known agent is toxic, it is desirable to limit the dosages administered in all cases, and particularly in those cases were drug resistance has increased the requisite dosage. When the claimed compounds are co-administered with the known agent, they reduce the dosage required which, in turn, reduces the deleterious effects. Further, because the claimed compounds are themselves both effective and non-toxic in large doses, co-administration of proportionally more of these compounds than known toxic therapeutics will achieve the desired effects while minimizing toxic effects.

V. Mitochondrial ATP Synthase (Mitochondrial F₀F₁ ATPase) Activity Modulators

In particularly preferred embodiments, the compositions (e.g., benzodiazepine derivatives) of the present invention provide therapeutic benefits to patients suffering from any one or more of a number of conditions (e.g., diseases characterized by dysregulation of necrosis and/or apoptosis processes in a cell or tissue, disease characterized by aberrant cell growth and/or hyperproliferation, etc.) by modulating (e.g., inhibiting or promoting) the activity of the mitochondrial ATP synthase (as referred to as mitochondrial F₀F₁ ATPase) complexes in affected cells or tissues. In particularly preferred embodiments, the compositions of the present invention inhibit the activity of mitochondrial ATP synthase complex by binding to a specific subunit of this multi-subunit protein complex. While the present invention is not limited to any particular mechanism, nor to any understanding of the action of the agents being administered, in some embodiments, the compositions of the present invention bind to the oligomycin sensitivity conferring protein (OSCP) portion of the mitochondrial ATP synthase complex. Likewise, it is further contemplated that when the compositions of the present invention bind to the OSCP the initial affect is overall inhibition of the mitochondrial ATP synthase complex, and that the downstream consequence of binding is a change in ATP level and the production of reactive oxygen species (e.g., O₂—). In still other preferred embodiments, while the present invention is not limited to any particular mechanism, nor to any understanding of the action of the agents being administered, it is contemplated that the generation of free radicals ultimately results in cell killing. In yet other embodiments, while the present invention is not limited to any particular mechanism, nor to any understanding of the action of the agents being administered, it is contemplated that the inhibiting mitochondrial ATP synthase complex using the compositions and methods of the present invention provides therapeutically useful inhibition of cell proliferation.

Accordingly, preferred methods embodied in the present invention, provide therapeutic benefits to patients by providing compounds of the present invention that modulate (e.g., inhibiting or promoting) the activity of the mitochondrial ATP synthase complexes in affected cells or tissues via binding to the oligomycin sensitivity conferring protein (OSCP) portion of the mitochondrial ATP synthase complex. Importantly, by itself the OSCP has no biological activity.

Thus, in one broad sense, preferred embodiments of the present invention are directed to the discovery that many diseases characterized by dysregulation of necrosis and/or apoptosis processes in a cell or tissue, or diseases characterized by aberrant cell growth and/or hyperproliferation, etc., can be treated by modulating the activity of the mitochondrial ATP synthase complex including, but not limited to, by binding to the oligomycin sensitivity conferring protein (OSCP) component thereof. The present invention is not intended to be limited, however, to the practice of the compositions and methods explicitly described herein. Indeed, those skilled in the art will appreciate that a number of additional compounds not specifically recited herein (e.g., non-benzodiazepine derivatives) are suitable for use in the methods disclosed herein of modulating the activity of mitochondrial ATP synthase.

The present invention thus specifically contemplates that any number of suitable compounds presently known in the art, or developed later, can optionally find use in the methods of the present invention. For example, compounds including, but not limited to, oligomycin, ossamycin, cytovaricin, apoptolidin, bafilomyxcin, and dicyclohexylcarbodiimide (DCCD), and the like, find use in the methods of the present invention. The present invention is not intended, however, to be limited to the methods or compounds specified above. In one embodiment, that compounds potentially useful in the methods of the present invention may be selected from those suitable as described in the scientific literature. (See e.g., K. B. Wallace and A. A. Starkov, Annu. Rev. Pharmacol. Toxicol., 40:353-388 [2000]; A. R. Solomon et al., Proc. Nat. Acad. Sci. U.S.A., 97(26):14766-14771 [2000]).

In some embodiments, compounds potentially useful in methods of the present invention are screened against the National Cancer Institute's (NCI-60) cancer cell lines for efficacy. (See e.g., A. Monks et al., J. Natl. Cancer Inst., 83:757-766 [1991]; and K. D. Paull et al., J. Natl. Cancer Inst., 81:1088-1092 [1989]). Additional screens suitable screens (e.g., autoimmunity disease models, etc.) are within the skill in the art.

In one aspect, derivatives (e.g., pharmaceutically acceptable salts, analogs, stereoisomers, and the like) of the exemplary compounds or other suitable compounds are also contemplated as being useful in the methods of the present invention.

Those skilled in the art of preparing pharmaceutical compounds and formulations will appreciate that when selecting optional compounds for use in the methods disclosed herein, that suitability considerations include, but are not limited to, the toxicity, safety, efficacy, availability, and cost of the particular compounds.

VI. Drug Screens

In preferred embodiments of the present invention, the compounds of the present invention, and other potentially useful compounds, are screened for their binding affinity to the oligomycin sensitivity conferring protein (OSCP) portion of the mitochondrial ATP synthase complex. In particularly preferred embodiments, compounds are selected for use in the methods of the present invention by measuring their biding affinity to recombinant OSCP protein. A number of suitable screens for measuring the binding affinity of drugs and other small molecules to receptors are known in the art. In some embodiments, binding affinity screens are conducted in in vitro systems. In other embodiments, these screens are conducted in in vivo or ex vivo systems. While in some embodiments quantifying the intracellular level of ATP following administration of the compounds of the present invention provides an indication of the efficacy of the methods, preferred embodiments of the present invention do not require intracellular ATP level quantification.

Additional embodiments are directed to measuring levels (e.g., intracellular) of superoxide in cells and/or tissues to measure the effectiveness of particular contemplated methods and compounds of the present invention. In this regard, those skilled in the art will appreciate and be able to provide a number of assays and methods useful for measuring superoxide levels in cells and/or tissues.

In some embodiments, structure-based virtual screening methodologies are contemplated for predicting the binding affinity of compounds of the present invention with OSCP.

Any suitable assay that allows for a measurement of the rate of binding or the affinity of a benzodiazepine or other compound to the OSCP may be utilized. Examples include, but are not limited to, surface plasma resonace (SPR) and radio-immunopreciptiation assays (Lowman et al., J. Biol. Chem. 266:10982 [1991]). Surface Plasmon Resonance techniques involve a surface coated with a thin film of a conductive metal, such as gold, silver, chrome or aluminum, in which electromagnetic waves, called Surface Plasmons, can be induced by a beam of light incident on the metal glass interface at a specific angle called the Surface Plasmon Resonance angle. Modulation of the refractive index of the interfacial region between the solution and the metal surface following binding of the captured macromolecules causes a change in the SPR angle which can either be measured directly or which causes the amount of light reflected from the underside of the metal surface to change. Such changes can be directly related to the mass and other optical properties of the molecules binding to the SPR device surface. Several biosensor systems based on such principles have been disclosed (See e.g., WO 90/05305). There are also several commercially available SPR biosensors (e.g., BiaCore, Uppsala, Sweden).

In some embodiments, benzodiazepine compounds are screened in cell culture or in vivo (e.g., non-human or human mammals) for their ability to modulate mitochondrial ATP synthase activity. Any suitable assay may be utilized, including, but not limited to, cell proliferation assays (Commercially available from, e.g., Promega, Madison, Wis. and Stratagene, La Jolla, Calif.) and cell based dimerization assays. (See e.g., Fuh et al., Science, 256:1677 [1992]; Colosi et al., J. Biol. Chem., 268:12617 [1993]). Additional assay formats that find use with the present invention include, but are not limited to, assays for measuring cellular ATP levels, and cellular superoxide levels.

The present invention also provides methods of modifying and derivatizing the compositions of the present invention to increase desirable properties (e.g., binding affinity, activity, and the like), or to minimize undesirable properties (e.g., nonspecific reactivity, toxicity, and the like). The principles of chemical derivatization are well understood. In some embodiments, iterative design and chemical synthesis approaches are used to produce a library of derivatized child compounds from a parent compound. In other embodiments, rational design methods are used to predict and model in silico ligand-receptor interactions prior to confirming results by routine experimentation.

### EXAMPLES

The following examples are provided to demonstrate and further illustrate certain preferred embodiments of the present invention and are not to be construed as limiting the scope thereof.

### Example 1

### Preparation of Compounds

The benzodiazepine compounds are prepared using either solid-phase or soluble-phase combinatorial synthetic methods as well as on an individual basis from well-established techniques. (See e.g., C. J. Boojamra et al., J. Org. Chem., 62:1240-1256 [1996]); B. A. Bunin et al., Proc. Natl. Acad. Sci. USA, 91:4708-4712 [1994]; S. Y. Stevens et al., J. Am. Chem. Soc., 118:10650-10651 [1996]; E. M. Gordon et al., J. Med. Chem., 37:51385-1401 [1994]; and U.S. Pat. Nos. 4,110,337 and 4,076,823, which are all incorporated by reference herein in their entirety. For illustration, the following general methodologies are provided.

Preparation of 1,4-benzodiazepine-2-one Compounds

Improved solid-phase synthetic methods for the preparation of a variety of 1,4-benzodiazepine-2-one derivatives with very high overall yields have been reported in the literature. (See e.g., Bunin and Ellman, J. Am. Chem. Soc., 114:10997-10998 [1992]). Using these improved methods, the 1,4-benzodiazepine-2-ones is constructed on a solid support from three separate components: 2-aminobenzophenones, α-amino acids, and (optionally) alkylating agents.

Preferred 2-aminobenzophenones include the substituted 2-aminobenzophenones, for example, the halo-, hydroxy-, and halo-hydroxy-substituted 2-aminobenzophenones, such as 4-halo-4′-hydroxy-2-aminobenzophenones. A preferred substituted 2-aminobenzophenone is 4-chloro-4′-hydroxy-2-aminobenzophenone. Preferred α-amino acids include the 20 common naturally occurring α-amino acids as well as α-amino acid mimicking structures, such as homophenylalanine, homotyrosine, and thyroxine.

Alkylating agents include both activated and inactivated electrophiles, of which a wide variety are well known in the art. Preferred alkylating agents include the activated electrophiles p-bromobenzyl bromide and t-butyl-bromoacetate.

In the first step of such a synthesis, the 2-aminobenzophenone derivative is attached to a solid support, such as a polystyrene solid support, through either a hydroxy or carboxylic acid functional group using well known methods and employing an acid-cleavable linker, such as the commercially available [4-(hydroxymethyl)phenoxy]acetic acid, to yield the supported 2-aminobenzophenone. (See e.g., Sheppard and Williams, Intl. J. Peptide Protein Res., 20:451-454 [1982]). The 2-amino group of the aminobenzophenone is preferably protected prior to reaction with the linking reagent, for example, by reaction with FMOC-C1 (9-fluorenylmethyl chloroformate) to yield the protected amino group 2′-NHFMOC.

In the second step, the protected 2-amino group is deprotected (for example, the —NHFMOC group may be deprotected by treatment with piperidine in dimethylformamide (DMF)), and the unprotected 2-aminobenzophenone is then coupled via an amide linkage to an α-amino acid (the amino group of which has itself been protected, for example, as an —NHFMOC group) to yield the intermediate. Standard activation methods used for general solid-phase peptide synthesis are used (such as the use of carbodiimides and hydroxybentzotriazole or pentafluorophenyl active esters) to facilitate coupling. However, a preferred activation method employs treatment of the 2-aminobenzophenone with a methylene chloride solution of the of α-N-FMOC-amino acid fluoride in the presence of the acid scavenger 4-methyl-2,6-di-tert-butylpyridine yields complete coupling via an amide linkage. This preferred coupling method has been found to be effective even for unreactive aminobenzophenone derivatives, yielding essentially complete coupling for derivatives possessing both 4-chloro and 3-carboxy deactivating substituents.

In the third step, the protected amino group (which originated with the amino acid) is first deprotected (e.g., —NHFMOC may be converted to —NH₂ with piperidine in DMF), and the deprotected Bz-423s reacted with acid, for example, 5% acetic acid in DMF at 60° C., to yield the supported 1,4-benzodiazepine derivative. Complete cyclization has been reported using this method for a variety of 2-aminobenzophenone derivatives with widely differing steric and electronic properties.

In an optional fourth step, the 1,4-benzodiazepine derivative is alkylated, by reaction with a suitable alkylating agent and a base, to yield the supported fully derivatized 1,4-benzodiazepine. Standard alkylation methods, for example, an excess of a strong base such as LDA (lithium diisopropylamide) or NaH, is used; however, such methods may result in undesired deprotonation of other acidic functionalities and over-alkylation. Preferred bases, which may prevent over-alkylation of the benzodiazepine derivatives (for example, those with ester and carbamate functionalities), are those which are basic enough to completely deprotonate the anilide functional group, but not basic enough to deprotonate amide, carbamate or ester functional groups. An example of such a base is lithiated 5-(phenylmethyl)-2-oxaxolidinone, which is reacted with the 1,4-benzodiazepine in tetrahydrofuran (THF) at −78° C. Following deprotonation, a suitable alkylating agent, as described above, is added.

In the final step, the fully derivatized 1,4-benzodiazepine is cleaved from the solid support. This is achieved (along with concomitant removal of acid-labile protecting groups), for example, by exposure to a suitable acid, such as a mixture of trifluoroacetic acid, water, and dimethylsulfide (85:5:10, by volume). Alternatively, the above benzodiazepines is prepared in soluble phase. The synthetic methodology was outlined by Gordon et al., J. Med. Chem., 37:1386-1401 [1994]) which is hereby incorporated by reference. Briefly, the methodology comprises trans-imidating an amino acid resin with appropriately substituted 2-aminobenzophenone imines to form resin-bound imines. These imines are cyclized and tethered by procedures similar to those in solid-phase synthesis described above. The general purity of benzodiazepines prepared using the above methodology is about 90% or higher.

Preparation of 1,4-benzodiazepine-2,5-diones

A general method for the solid-phase synthesis of 1,4-benzodiazepine-2,5-diones has been reported in detail by C. J. Boojamra et al., J. Org. Chem., 62:1240-1256 [1996]). This method is used to prepare the compounds of the present invention.

A Merrifield resin, for example, a (chloromethyl)polystyrene is derivatized by alkylation with 4-hydroxy-2,6-dimethoxybenzaldehyde sodium to provide resin-bound aldehyde. An α-amino ester is then attached to the derivatized support by reductive amination using NaBH(OAc)₃ in 1% acetic acid in DMF. This reductive amination results in the formation of a resin-bound secondary amine.

The secondary amine is acylated with a wide variety of unprotected anthranilic acids result in support-bound tertiary amides. Acylation is best achieved by performing the coupling reaction in the presence of a carbodiimide and the hydrochloride salt of a tertiary amine. One good coupling agent is l-ethyl-8-[8-(dimethylamino)propyl] carbodiimide hydrochloride. The reaction is typically performed in the presence of anhydrous 1-methyl-2-pyrrolidinone. The coupling procedure is typically repeated once more to ensure complete acylation.

Cyclization of the acyl derivative is accomplished through base-catalyzed lactamation through the formation of an anilide anion which would react with an alkylhalide for simultaneous introduction of the substituent at the 1-position on the nitrogen of the heterocyclic ring of the benzodiazepine. The lithium salt of acetanilide is a good base to catalyze the reaction. Thus, the Bz-423s reacted with lithium acetanilide in DMF/THF (1:1) for 30 hours followed by reaction with appropriate alkylating agent provides the fully derivatized support-bound benzodiazepine. The compounds are cleaved from the support in good yield and high purity by using TFA/DMS/H₂O (90:5:5).

Some examples of the α-amino ester starting materials, alkylating agents, and anthranilic acid derivatives that are used in the present invention are listed by C. J. Boojamra et al., J. Org. Chem., 62:1240-1256 [1996], supra at 1246. Additional reagents are readily determined and either are commercially obtained or readily prepared by one of ordinary skill in the art to arrive at the novel substituents disclosed in the present invention.

For example, from Boojamra, supra, one realizes that: alkylating agents provide the R₁ substituents; α-amino ester starting materials provide the R₂ substituents, and anthranilic acids provide the R₄ substituents. By employing these starting materials that are appropriately substituted, one arrives at the desired 1,4-benzodiazepine-2,5-dione. The R₃ substituent is obtained by appropriately substituting the amine of the α-aminoester starting material. If steric crowding becomes a problem, the R₃ substituent is attached through conventional methods after the 1,4-benzodiazepine-2,5-dione is isolated.

### Example 2

### Chirality

It should be recognized that many of the benzodiazepines of the present invention exist as optical isomers due to chirality wherein the stereocenter is introduced by the α-amino acid and its ester starting materials. The above-described general procedure preserves the chirality of the α-amino acid or ester starting materials. In many cases, such preservation of chirality is desirable. However, when the desired optical isomer of the α-amino acid or ester starting material is unavailable or expensive, a racemic mixture is produced which is separated into the corresponding optical isomers and the desired benzodiazepine enantiomer is isolated.

For example, in the case of the 2,5-dione compounds, Boojamra, supra, discloses that complete racemization is accomplished by preequilibrating the hydrochloride salt of the enantiomerically pure α-amino ester starting material with 0.3 equivalents of i-Pr₂EtN and the resin-bound aldehyde for 6 hours before the addition of NaBH(OAc)₃. The rest of the above-described synthetic procedure remains the same. Similar steps are employed, if needed, in the case of the 1,4-benzodiazepine-2-dione compounds as well.

Methods to prepare individual benzodiazepines are well-known in the art. (See e.g., U.S. Pat. Nos. 3,415,814; 3,384,635; and 3,261,828, which are hereby incorporated by reference). By selecting the appropriately substituted starting materials in any of the above-described methods, the benzodiazepines of this invention are prepared with relative ease.

### Example 3

### Reagents

Bz-423 is synthesized as described above. FK506 is obtained from Fujisawa (Osaka, Japan). N-benzoylcarbonyl-Val-Ala-Asp-fluoromethylketone (z-VAD) is obtained from Enzyme Systems (Livermore, CA). Dihydroethidium (DHE) and 3,3′-dihexyloxacarbocyanine iodide (DiOC₆(3)) are obtained from Molecular Probes (Eugene, Oreg.). FAM-VAD-fink is obtained from Intergen (Purchase, N.J.). Manganese(III)meso-tetrakis(4-benzoic acid)porphyrin (MnTBAP) is purchased from Alexis Biochemicals (San Diego, Calif.). Benzodiazepines is synthesized as described (See, B. A. Bunin et al., Proc. Natl. Acad. Sci. U.S.A., 91:4708-4712 [1994]). Other reagents were obtained from Sigma (St. Louis, Mo.).

### Example 4

### Animals and Drug Delivery

Female NZB/W mice (Jackson Labs, Bar Harbor, Me.) are randomly distributed into treatment and control groups. Control mice receive vehicle (50 μL aqueous DMSO) and treatment mice receive Bz-423 dissolved in vehicle (60 mg/kg) through intraperitoneal injections. Peripheral blood is obtained from the tail veins for the preparation of serum. Samples of the spleen and kidney are preserved in either 10% buffered-formalin or by freezing in OCT. An additional section of spleen from each animal is reserved for the preparation of single cell suspensions.

### Example 5

### Primary Splenocytes, Cell Lines, and Culture Conditions

Primary splenocytes are obtained from 6 month old mice by mechanical disruption of spleens with isotonic lysis of red blood cells. B cell-rich fractions are prepared by negative selection using magnetic cell sorting with CD4, CD8a and CD11b coated microbeads (Miltenyi Biotec, Auburn, Calif.). The Ramos line is purchased from the ATCC (Monassis, Ga.). Cells are maintained in RPMI supplemented with 10% heat-inactivated fetal bovine serum (FBS), penicillin (100 U/ml), streptomycin (100 μg/ml) and L-glutamine (290 μg/ml). Media for primary cells also contains 2-mercaptoethanol (50 μM). All in vivo studies are performed with 0.5% DMSO and 2% FBS. In vitro experiments are conducted in media containing 2% FBS. Organic compounds are dissolved in media containing 0.5% DMSO.

### Example 6

### Histology

Formalin-fixed kidney sections were stained with hematoxylin and eosin (H&E) and glomerular immune-complex deposition is detected by direct immunofluorescence using frozen tissue stained with FITC-conjugated goat anti-mouse IgG (Southern Biotechnology, Birmingham, Ala.). Sections are analyzed in a blinded fashion for nephritis and IgG deposition using a 0-4+ scale. The degree of lymphoid hyperplasia is scored on a 0-4+ scale using spleen sections stained with H&E. To identify B cells, sections are stained with biotinylated-anti-B220 (Pharmingen; 1 μg/mL) followed by streptavidin-Alexa 594 (Molecular Probes; 5 μg/mL). Frozen spleen sections are analyzed for TUNEL positive cells using an In situ Cell Death Detection kit (Roche) and are evaluated using a 0-4+ scale.

### Example 7

### TUNEL Staining

Frozen spleen sections are analyzed using an In situ Cell Death Detection kit (Roche Molecular Biochemicals, Indianapolis, Ind.). Sections are blindly evaluated and assigned a score (0-4+) on the basis of the amount of TUNEL-positive staining.

B cells are identified by staining with biotinylated-anti-B220 (Pharmingen, San Diego, Calif.; 1 μg/mL, 1 h, 22° C.) followed by streptavidin-Alexa 594 (Molecular Probes, Eugene, Oreg.; 5 μg/mL, 1 h, 22° C.).

### Example 8

### Flow Cytometric Analysis of Spleen Cells from Treated Animals

Surface markers are detected (15 m, 4° C.) with fluorescent-conjugated anti-Thy 1.2 (Pharmingen, 1 μg/mL) and/or anti-B220 (Pharmingen, 1 μg/mL). To detect outer-membrane phosphatidyl serine, cells are incubated with FITC-conjugated Annexin V and propidium iodide (PI) according to manufacturer protocols (Roche Molecular Biochemicals). Detection of TUNEL-positive cells by flow cytometry uses the APO-BRDU kit (Pharmingen). Superoxide and MPT are assessed by incubation of cells for 30 m at 27° C. with 10 μM dihydroethidium and 2 μM 3,3′-dihexyloxacarbocyanine iodide (DIOC₆(3)) (Molecular Probes). Prodidium idodie is used to determine viability and DNA content. Samples are analyzed on a FACSCalibur flow cytometer (Becton Dickinson, San Diego, Calif.).

### Example 9

### B Cell Stimulation

Ramos cells are activated with soluble goat Fab₂ anti-human IgM (Southern Biotechnology Associates, 1 μg/ml) and/or purified anti-human CD40 (Pharmingen, clone 5C3, 2.5 μg/ml). Mouse B cells are activated with affinity purified goat anti-mouse IgM (ICN, Aurora, Ohio; 20 μg/ml) immobilized in culture wells, and/or soluble purified anti-mouse CD40 (Pharmingen, clone HM40-3, 2.5 μg/ml). LPS is used at 10 μg/ml. Bz-423 is added to cultures immediately after stimuli are applied. Inhibitors are added 30 m prior to Bz-423.

### Example 10

### Statistical Analysis

Statistical analysis is conducted using the SPSS software package. Statistical significance is assessed using the Mann-Whitney U test and correlation between variables is assessed by two-way ANOVA. All p-values reported are one-tailed and data are presented as mean±SEM.

### Example 11

### Detection of Cell Death and Hypodiploid DNA

Cell viability is assessed by staining with propidium iodide (PI, 1 μg/mL). PI fluorescence is measured using a FACScalibur flow cytometer (Becton Dickinson, San Diego, Calif.). Measurement of hypodiploid DNA is conducted after incubating cells in DNA-labeling solution (50 μg/mL of PI in PBS containing 0.2% Triton and 10 μg/mL RNAse A) overnight at 4° C. The data is analyzed using the CellQuest software excluding aggregates.

### Example 12

### Detection of O₂⁻, ΔΨₘ, and Caspase Activation

To detect O₂⁻, cells are incubated with DHE (10 μM) for 30 min at 37° C. and are analyzed by flow cytometry to measure ethidium fluorescence. Flow analysis of mitochondrial transmembrane potential (ΔΨₘ) is conducted by labeling cells with DiOC₆(3) (20 nM) for 15 min at 37° C. A positive control for disruption of ΔΨₘ is established using carbonyl cyanide m-chlorophenylhydrazone (CCCP, 50 μM). Caspase activation assays are performed with FAM-VAD-fluoromethylketone. Processing of the substrate is evaluated by flow cytometry.

### Example 13

### Subcellular Fractionation and Cytochrome c Detection

Ramos cells (250×10⁶ cells/sample) are treated with Bz-423 (10 μM) or vehicle for 1 to 5 h. Cells are pelleted, re-suspended in buffer (68 mM sucrose, 220 mM mannitol, 10 mM HEPES-NaOH, pH 7.4, 10 mM KCl, 1 mM EDTA, 1 mM EGTA, 10 μg/mL leupeptin, 10 μg/mL aprotinin, 1 mM PMSF), incubated on ice for 10 min, and homogenized. The homogenate is centrifuged twice for 5 min at 4° C. (800 g) to pellet nuclei and debris and for 15 min at 4° C. (16,000 g) to pellet mitochondria. The supernatant is concentrated, electrophoresed on 12% SDS-PAGE gels, and transferred to Hybond ECL membranes (Amersham, Piscataway, N.J.). After blocking (PBS containing 5% dried milk and 0.1% Tween), the membranes are probed with an anti-cytochrome c monoclonal antibody (Pharmingen, San Diego, Calif.; 2 μg/mL) followed by an anti-mouse horseradish peroxidase-conjugated secondary with detection by chemiluminescence (Amersham).

### Example 14

### ROS Production in Isolated Mitochondria

Male Long Evans rats are starved overnight and sacrificed by decapitation. Liver samples are homogenized in ice cold buffer A (250 mM sucrose, 10 mM Tris, 0.1 mM EGTA, pH 7.4), and nuclei and cellular debris are pelleted (10 min, 830 g, 4° C.). Mitochondria are collected by centrifugation (10 min, 15,000 g, 4° C.), and the supernatant is collected as the S15 fraction. The mitochondrial pellet is washed three times with buffer B (250 mM sucrose, 10 mM Tris, pH 7.4), and re-suspended in buffer B at 20-30 mg/mL. Mitochondria are diluted (0.5 mg/mL) in buffer C (200 mM sucrose, 10 mM Tris, pH 7.4, 1 mM KH₂PO₄, 10 μM EGTA, 2.5 μM rotenone, 5 mM succinate) containing 2′,7′-dichlorodihydrofluorescin diacetate (DCFH-DA, 1 μM). For state 3 measurements, ADP (2 mM) is included in the buffer, and prior to the addition of Bz-423, mitochondria are allowed to charge for 2 min. To induce state 4, oligomycin (10 μM) is added to buffer C. The oxidation of DCFH to 2′,7′-dichlorofluorescein (DCF) is monitored at 37° C. with a spectrofluorimeter (λₑₓ: 503 nm; λₑₘ: 522 nm). To detect effects on O₂⁻ and ΔΨₘ, mitochondria are incubated for 15 min at 37° C. in buffer C with vehicle, Bz-423, or CCCP containing DHE (5 μM) or DIOC₆(3) (20 nM), and aliquots are removed for analysis by fluorescence microscopy.

### Example 15

### Flow Cytometric Analysis of Splenocytes

Splenocytes are prepared by mechanical disruption and red blood cells removed by isotonic lysis. Cells are stained at 4° C. with fluorescent-conjugated anti-Thy 1.2 (Pharmingen; 1 μg/mL) and/or anti-B220 (Pharmingen; 1 μg/mL) for 15 min. To detect outer-membrane phosphatidyl serine, cells are incubated with FITC-conjugated Annexin V and PI (Roche Molecular Biochemicals, Indianapolis, Ind.; 1 μg/mL).

### Example 16

### In vivo Determination of ROS

Spleens are removed from 4-mo old NZB/W mice treated with Bz-423 or vehicle and frozen in OCT. ROS production is measured using manganese(II)3,3,9-diaminobenzidine as described in E. D. Kerver et al. (See, E. D. Kerver et al., Histochem. J., 29:229-237 [1997).

### Example 17

### IgG Titers, BUN, and Proteinuria

Anti-DNA and IgG titers are determined by ELISA as described in P. C. Swanson et al. (See, P. C. Swanson et al., Biochemistry, 35:1624-1633 [1996]). Serum BUN is measured by the University of Michigan Hospital's clinical laboratory. Proteinuria is monitored using ChemStrip 6 (Boehringer Mannheim).

### Example 18

### Benzodiazepine Studies

General Methods

Cell Preparation:

Cell lines were cultured in complete media (RPMI or DMEM containing 10% fetal bovine serum supplemented with penicillin, streptomycin, and L-glutamine) at 37° C., 5% CO₂. For activity assays, cells in log-phase growth were removed and diluted to a concentration between 100,000 and 300,000/mL. Some cells were kept in complete media, while an identical aliquot was exchanged into reduced serum media (RPMI or DMEM containing 0.2% fetal bovine serum supplemented with penicillin, streptomycin, and L-glutamine) by centrifugation.

Activity Assays:

Cells in both complete media and reduced serum media were dispensed into 96 well plates in 100 μL aliquots giving 10,000 to 30,000 cells/well. Compound was then added to appropriate wells in the plate (2 μL of each 50× stock) at concentrations between 1 nM to 20 μM. Cells were then cultured overnight 37° C., 5% CO₂). Relative cell number/cell viability was measured using standard techniques (trypan blue exclusion/hemocytometry, MTT dye conversion assay).

### Example 18.1

### Ability to Induce Cell Death

Several individual representative compounds that induce apoptosis have been shown above. Of these, the most potent Bz-423s identified as Bz-423, which is shown below.

Bz-423 was used to induce cell death in a variety of cells by using the above-described materials and methods. Table 3 shows cell viability data after 18 hours of culture with Bz-423 in reduced serum media as described above.

TABLE 3

| | | | | | 10 μM |
|---|---|---|---|---|---|
| Cell Line | Source | Type | 4 μM Bz | 6 μM Bz | Bz |
| Jurkat | Human | T-cell | 0 | 0 | 0 |
| IMR | Human | Neuroblastoma | 100 | 0 | 0 |
| SHSY-5Y | Human | Neuroblastoma | Nd | | 0 |
| Shep | Human | Neuroblastoma | Nd | 80 | 0 |
| 293T | Human | Embryonic | Nd | Nd | 30 |
| | | fibroblast | | | |
| RAW 246.7 | Murine | monocytic | 70% | 0 | 0 |
| NIH 3T3 | Murine | fibroblast | Nd | Nd | 25 |

(lower numbers equal increased killing).
Nd = Not determined

### Example 18.2

MRL/MpJ-lpr/lpr (MRL-lpr) mice develop similar serological and histological manifestations of autoimmune disease as human SLE. These mice were developed by a series of cross-breeding of inbred strains until an autoimmune phenotype appeared. (A. N. Theofilopoulos and F. J. Dixon, Adv. Immunol., 37:269-390 [1985]). The MRL-lpr mice are characterized by the spontaneous development of systemic autoimmune disease. This disease is manifested in several physiological locations and resembles a variety of human diseases. For instance, the kidney damage in these mice is associated with high serological titers of anti-DNA as in human SLE. They also develop an erosive arthropathy and a lymphocytic infiltration of the salivary glands, similar to the human diseases rheumatoid arthritis (RA) and Sjörgen's disease, respectively (Theofilopoulos, supra).

In general MRL-lpr mice have a profound defect in apoptosis due the mutation of the lpr gene locus. (K. Sakata et al., Clin. Immunol. Immunopathol. 87:1-7 [1998]). The defect has been linked to a mutation in the Fas receptor gene, important in the signaling of apoptosis in activated lymphocytes. (R. Watanabe-Fukunaga et al., Nature, 356:314-317 [1992]). Consequently, these mice show a profound lymphoproliferation resulting in massive enlargement of the lymph nodes and spleen. Grossly, these mice demonstrate swollen footpads and erythematous skin lesions. Histologically, glomerulonephritis, arthritis, and inflammatory infiltration of the salivary glands are notable.

Methods

Mice:

Six week old, female MRL-lpr mice were purchased from Jackson Laboratories (Bar Harbor, Me.). The animals were allowed to adapt to their environment for one week prior to commencement of the treatment study. The mice were housed in a climate controlled specific pathogen-free environment on a 12 hour light dark cycle with food and water ad libitum. Once a week, weights were measured and proteinuria, was examined using a calorimetric reaction (Boehringer Mannheim ChemStrip 6).

Treatment Regimen:

Mice were randomized into three groups: controls receiving PBS (50 μL, qod), controls receiving DMSO (50 μL, qod), and mice receiving Bz-423 in 50 μL of DMSO (60 mg/kg qod ip for 20 mice and 30 mg/kg qod ip for 10 mice). Intraperitoneal injections were given with a 30 G needle and glass syringes (Hamilton) on an every other day dosing schedule. Treatment started at 7 weeks of age for the control mice (those receiving PBS and DMSO) and at 8 or 9 weeks for the treatment mice. At the end of the study, blood was collected by tail bleeds. The mice were subsequently anesthetized by metophane inhalation and were sacrificed by exsanguination by axillary dissection. Sample organs were then removed for histological analysis.

Statistical Analysis:

Analysis of statistical significance was done using the computer program SPSS. Unless otherwise noted, the Mann-Whitney U test (one-tailed) was used and probability values >5% were considered insignificant.

Results

Disease Progression.

MRL-lpr mice are known to develop a kidney disease very similar to that seen in the human autoimmune disease Systemic Lupus Erythematosus (SLE). The hallmark of this disease is a glomerulonephritis that results in loss of kidney function and eventual death due to kidney failure. A marker for the development of kidney disease is the amount of protein present in the urine. As kidney function deteriorates, the glomerular filtration mechanism fails and proteinuria increases. Unlike the periodicity of the human disease, the murine form of lupus is progressive; thus, once a mouse develops nephritis and the ensuing proteinuria the disease progresses on a continuum until death. This allows the use of proteinuria measurements to follow the progression of kidney disease in the MRL-lpr mice.

The development of disease in our study was followed by weekly measurement of the proteinuria. Any given mouse was determined to have lupus if she had proteinuria values >2+(>100 mg/dL) for 2 or more consecutive weeks. No mice meeting this criterion were ever found to have drops in proteinuria below 2+. Furthermore, the mice that died with >2+ proteinuria were found to have very significant glomerulonephritis and the mice that died with values <2+ had healthy kidneys and causes of death unrelated to the autoimmune disease.

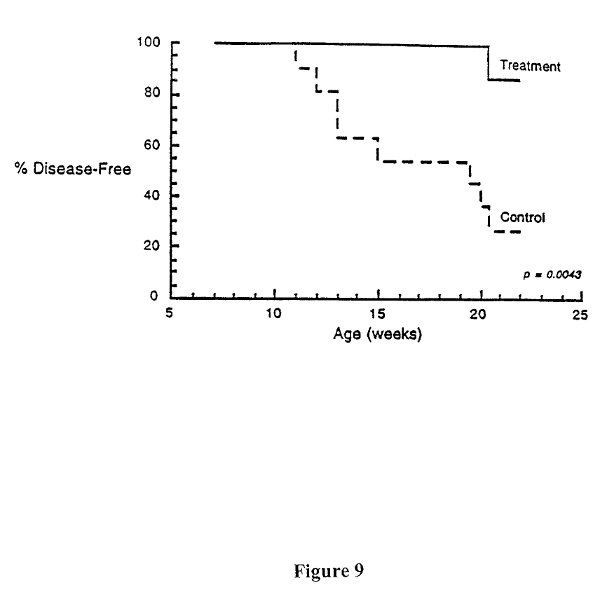

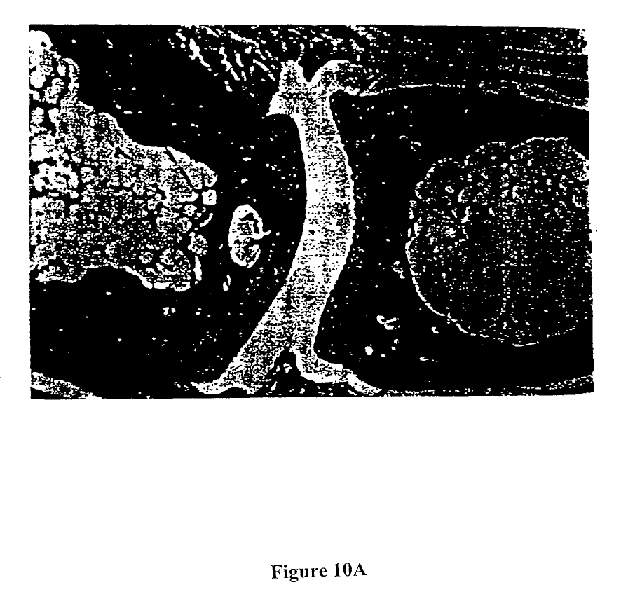

FIG. 9 provides disease progression analysis for mice treated 60 mg/kg qod. Similar data are obtained with the 30 mg/kg qod dosing schedule. As seen in FIG. 9, treating mice with Bz-423 significantly delays the onset of and effectively treats the lupus-like disease relative to the control mice (p=0.0043). These data are supported by the observation that BUN values for the treated animals are normal whereas those receiving vehicle alone are in renal distress.

Laboratory Diagnostics.

The blood from the animals was analyzed for alterations in total numbers of white blood cells (WBC) as well as differential representation of subtypes. Mice receiving Bz-423 (60 mg/kg) have nearly identical values for hematocrit, platelet count, and WBC relative to those receiving vehicle alone (Table 4).

TABLE 4

| Group | HCT | PLAT | WBC | POLYS | LYMPHS | MONO | EOS | BASO |
|---|---|---|---|---|---|---|---|---|
| Control | 46.70 | 809 | 13.22 | 31.50 | 65.07 | 2.35 | 0.88 | 0.13 |
| | (0.91) | (129) | (4.65) | (8.38) | (9.61) | (2.03) | (0.42) | (0.06) |
| Treatment | 42.60 | 895 | 13.68 | 43.00 | 53.66 | 2.01 | 1.15 | 0.17 |
| | (7.57) | (148) | (7.27) | (16.37) | (16.81) | (1.05) | (1.95) | 0.27 |
| P value | 0.267 | 0.183 | 0.5 | 0.069 | 0.069 | .0473 | 0.147 | 0.267 |

HCT, hematocrit (%);
PLAT, platelets (K/μL);
WBC, white blood cells (K/μL);
POLYS, polymorphonuclear cells (%);
LYMPHS, lymphocytes (%);
MONO, monocytes (%);
EOS, eosinophils
BASO, basophils (%).

Autoantibodies:

Serum samples from all of the mice were analyzed to determine the titer of antibodies to several autoantigens (Table 5). These antibodies are total serum polyclonal antibodies. At the termination of the study, the mice receiving Bz-423 showed significantly lower titers of antibodies to ssDNA (p=0.019), histones (p=0.0056), and La antigen (p=0.0265). Anti-dsDNA titers were lower in the treatment mice but not statistically different from those in the control animals (p=0.082). There was also no difference in antibodies to Ro antigen between the two groups of mice; however, the actual absorbance measurements were very low for these ELISAs and any differences may have been masked by the sensitivity of the assay. Anti-dsDNA titers were only observed in a few of the animals in both groups and no conclusions could be made regarding differences between the groups. These anti-Sm findings are consistent with the literature, which reports that only 10% of MRL-lpr mice are expected to be positive for antibodies against Sm antigen (Murphy, E. D. (1981). For data only lymphoproliferation lpr) and other single-locus models for murine lupus. (See, Immunologic Defects in Laboratory Animals (E. M. Gershwin and B. Merchant, eds.); Vol. 2, pp. 143-173 (Plenum, New York). The observed differences in autoantibody levels are found in a background of very high total IgG concentrations which do not differ statistically between the control and the treatment groups (p=0.3312).

TABLE 5

| | | Anti- | | | | |
|---|---|---|---|---|---|---|
| | Anti-ssDNA | dsDNA | Anti- | Total IgG | Anti-Rho | Anti-La |
| | (U/ml) | (U/ml) | Histone^{*} | (mg/ml) | (U/ml) | (U/ml) |
| Drug Group | 508 ± 193 | 247 ± 101 | 0.613 ± 0.526 | 23.3 ± 6.2 | 304 ± 256 | 226 ± 162 |
| Control | 887 ± 328 | 650 ± 454 | 1.387 ± 0.537 | 25.8 ± 8.7 | 456 ± 328 | 529 ± 462 |
| Group | | | | | | |
| P value | 0.019 | 0.082 | 0.0056 | 0.3312 | 0.1588 | 0.0265 |

^{*}Titers were not available at the time of this report. Anti-histone levels are reported as OD₄₀₅ values at a 1/400 dilution of serum.

Joint Histology:

In addition to the lupus syndrome, NIRL-lpr mice spontaneously develop a nerosive arthropathy that resembles human rheumatoid arthritis, both histologically and serologically. The arthritic lesions in these mice are characterized by inflammatory changes in the synovium and the periarticular connective tissue, frequently accompanied by the presence of circulating rheumatoid factors in the serum. This arthritic process is progressive and proceeds through several different stages from a mild synovitis to a nerosive arthritis, which can eventually lead to a scarred joint. Histologically, the majority of 5 month old MRL-lpr mice demonstrate synovial cell proliferation, destruction of articular cartilage and subchondral bone, infiltration of synovial stroma by inflammatory cells, periarticular inflammation (vasculitis, myositis, tendinitis, perineuritis), exudates, pannus formation, and subcutaneous fibrinoid nodules (L. Hang, et al., J. Exp. Med., 155:1690-1701 [1982]; W. J. Koopman and S. Gay, Scand. J. Rheumatology. Suppl., 75:284-289 [1988]).

The paws of all the mice treated with Bz-423 were examined for signs of arthritis and synovitis. The control mice (those receiving vehicle alone) have a severe synovitis characterized by a marked thickening of the synovium with occasional formation of papillary, villous configurations. Typically, the synovial pathology was a result of synovial cell proliferation and infiltration of the synovial stroma by inflammatory cells. In a substantial percentage of the control mice, the disease process was accompanied by pannus formation and erosion of the articular surface (both articular cartilage and subchondral bone). In contrast, the treatment mice were found to have a milder synovitis as well as fewer erosions and limited pannus formation (Table 6). The character of the disease in the animals receiving Bz-423 was much less aggressive with less synovial cell proliferation and inflammatory infiltration. Of further interest, it was observed that the treatment mice had a lessened degree of periarticular inflammation. The combination of these findings suggest that Bz-423 is ameliorating the arthritic disease process that typically destroys the joints of MPL-lpr mice.

TABLE 6

| | | Avg. | Number of | | Number of |
|---|---|---|---|---|---|
| | | histologic | mice with | Number of | mice with |
| | Number of | score | synovitis ≧ | mice with | pannus |
| Group | mice | synovitis | 2^{*} | erosions^{*} | formation^{*} |
| Control | 7 | 2.1 | 5 (71%) | 4 (57%) | 4 (57%) |
| Treatment | 7 | 1.3 | 0 (0%) | 1 (14%) | 1 (14%) |
| P value | | p = 0.001 | p = 0.01 | p = 0.13 | p = 0.13 |

^{*}p value determined by cross-tabulation and chi-square analysis

Delayed Type Hypersensitivity (DTH):

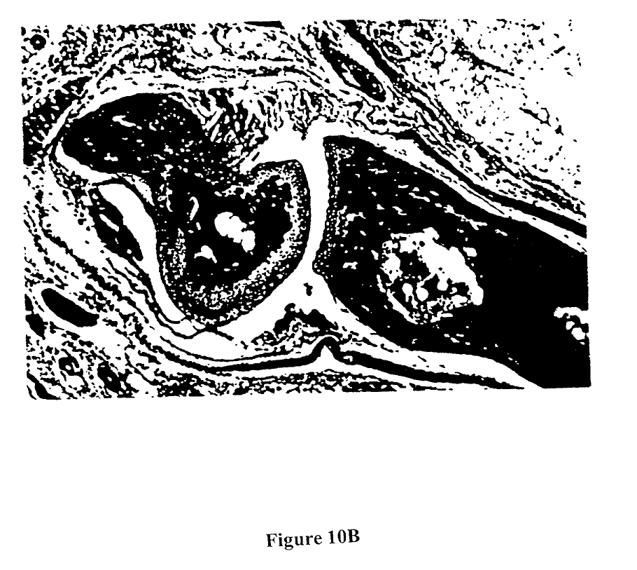

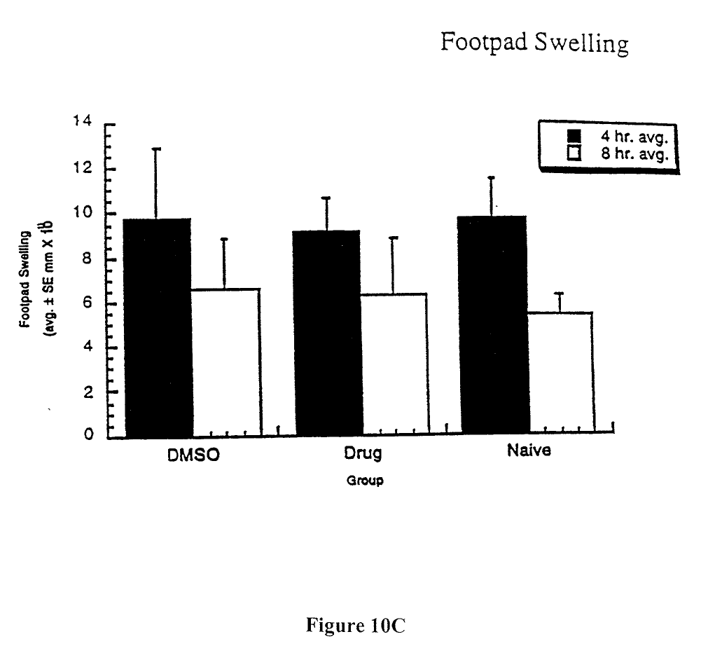

Mice treated with Bz-423 (60 mg/kg) showed no difference in DTH response to TNBS on comparison to the control mice. (FIG. 10C). Importantly, neither group of animals demonstrated a significant footpad swelling following antigen challenge. This phenomenon has been documented and old MRL-lpr mice (>10 weeks) are expected to have a diminished in vivo T cell response to stimulus as evidenced by the absence of a DTH response. (See e.g., H. Okuyama et al., Clin. Exp. Immunol., 63:87-94 [1986], C. F. Scott et al., J. Immunol., 132:633 [1984]). However, suppression of T cells can result in a rescue of the DTH response. (See, H. Okuyama et al., Intl. Arch. Allergy Appl. Immunol., 1588:394 [1989]). Such a rescue was not observed in this study's treatment protocol. These data suggest that Bz-423 does not alter T cell function. FIGS. 10A-10C. show the results of the Footpad Swelling experiment.

Immune Cell Function:

Thymidine uptake assays of using both stimulated and unstimulated T and B cells was conducted to determine if Bz-423 affects lymphoproliferation in vitro. At about a concentration of 10 μM, no effect on lymphocyte proliferation was observed.

### Example 18.3

### Bz-423 as a Lymphotoxic Agent

Methods

Animals and Experimental Design:

Female NZB/W mice (Jackson Labs) were housed in specific pathogen-free, environmentally controlled rooms operated by the University of Michigan's Unit for Laboratory Animal Medicine with 12 hr light-dark cycles and were given food and water ad libitum. Mice were randomly distributed into treatment and control groups. All mice were dosed through intraperitoneal injections using a Hamilton repeating dispenser with glass microliter syringes and 30 gauge needles. Control mice received vehicle (50 μL aqueous DMSO) and treatment mice received Bz-423 dissolved in vehicle. Animal weights were determined weekly, and dosing schedules readjusted thereafter.

Collection of Blood/Tissues:

Peripheral blood was obtained from the tail veins of all mice for complete blood counts analysis and collection of serum. Blood was first allowed to clot at room temperature for I h, and then overnight at 4° C. Serum was separated from the formed clot by centrifugation (6 min., 16,000×g). A section of spleen was removed aseptically for preparation of single cell suspensions. Samples of the following organs were preserved in IO% buffered-formalin: heart, liver, lung, spleen, kidney small intestine, reproductive system, salivary glands, thymus, mesenteric and axillary lymph nodes, and skin. Additional sections of kidney and spleen were preserved by snap-freezing in OCT. Bone marrow smears were prepared from each femur.

Histology:

All histological determinations were made in a blinded fashion by a pathologist. Formalin-fixed sections were cut and stained with hematoxylin and eosin (H&E) using standard protocols (L. G. Luna in: Manual of Histological Staining Methods of the Armed Forces Institute of Pathology, McGraw-Hill, New York (1960)). Immune-complex deposition in the kidneys was evaluated by direct immunofluorescence using frozen sections stained with FITC-conjugated goat anti-mouse IgG (Southern Biotechnology Associates, Birmingham, Ala.) and C3 (Cappel-Organon Teknika, Durham, N.C.). The degree of lymphoid hyperplasia was scored 0-4+scale.

TUNEL Staining:

Frozen spleen sections (4 μm thick) were assayed for DNA strand breaks using the In situ Cell Death Detection kit (Roche Molecular Biochemicals) according to the manufacturer's protocols. Sections were analyzed using a 0-4+ scale. Sections were blindly evaluated and assigned a score on the basis of the amount of TUNEL-positive staining.

Fluorescence Analysis of Lymphocyte Populations:

Single cell suspensions were prepared by teasing apart the spleen in media, followed by removal red blood cell with isotonic lysis buffer (A. M. Kruisbeek in: Current Protocols in Immunology, eds., J. E. Coligan et al., pp. 3.1.2-3.1.5, John Wiley & Sons, Inc., [1997]). 106 cells were stained at 4° C. with fluorescently-conjugated anti-Thy 1.2 (Pharmingen, clone: 53-2.1, 1, μg/mL) and/or anti-B220 (Pharmingen, clone: RA3-6B2 1, :g/mL) for 15 min. In samples stained to detect outer-membrane phosphatidyl serine, cells were then incubated with FITC-conjugated Annexin V and PI according to manufacturer protocols (Roche Molecular Biochemicals). Cells were analyzed on a Coulter ELITE flowcytometer. For each sample, at least 10,000 events were counted.

Serum Anti-DNA:

Titers were determined by direct ELISA as previously described (P. C. Swanson, et al., J. Clin. Invest., 97:1748-1760 [1996]). Detection of IgG anti-DNA used an alkaline phosphatase-conjugated Goat anti-Mouse IgG (H-chain only) secondary antibody (1/1000 dilution, SIGMA). To convert absorbance readings into titers, pooled serum from unmanipulated eight month old female NZB/W was used as a reference standard which was arbitrarily assigned a value of 1000 U/mL.

Serum Immunoglobulin:

Concentrations were determined by capture ELISA. Goat anti-Mouse Ig (Southern Biotechnology Associates) was diluted to 10 pg/mL in PBS and coated overnight at 4° C. on Immulon II microtiter plates. Otherwise, ELISAs were performed as previously described. To convert absorbances into concentrations, a standard curve was generated using a previously quantified mouse immunoglobulin reference serum (ICN Biomedicals, Aurora, Ohio).

Blood Urea Nitrogen (BUN) and Complete Blood Counts (CBC):

Serum BUN measurements were conducted by the University of Michigan Hospital's clinical pathology laboratory. CBC analyses were conducted by the diagnostic laboratory of University of Michigan's Unit for Laboratory Animal Medicine. Automated counts determined by a Hemavet 15 OR were confirmed by visual examination of blood smears.

Serum 1,4-benzodiazepine Levels:

Serum samples from mice injected with Bz-423 were precipitated with acetone (5× volume, −20° C., 10 min). After centrifugation (16,000×g, 10 min), the supernatant containing Bz-423 was concentrated in vacuo, and then extracted from any remaining protein using a Sep-pak C18 column (Waters Corp.) running a step gradient from 10% acetonitrile in water to 100% acetonitrile. Material eluting in the organic fraction was concentrated in vacuo, and then analyzed by reversed-phase BPLC using a Phenomenex C18 column. Peak areas were determined using a Shimadzu integrator and were referenced to a standard curve.

Statistical Analysis:

Statistical analyses was conducted using the SPSS software package. The Mann-Whitney U and chi-square tests were used for histological and clinical data. Student's t-test was used for flow cytometric data. Correlations were assessed by ANOVA.

### Example 18.4

To model neuroblastoma in mice, the mice were transfected with the human neuroblastoma cell line SKNAS, to cause the cells to overexpress the neuroblastoma associated human oncogene N-myc. The resulting cell line is designated as D2. ThesE cells form tumors when xenografted into T cell-deficient athymic mice; thus providing a relevant animal model of human neuroblastoma.

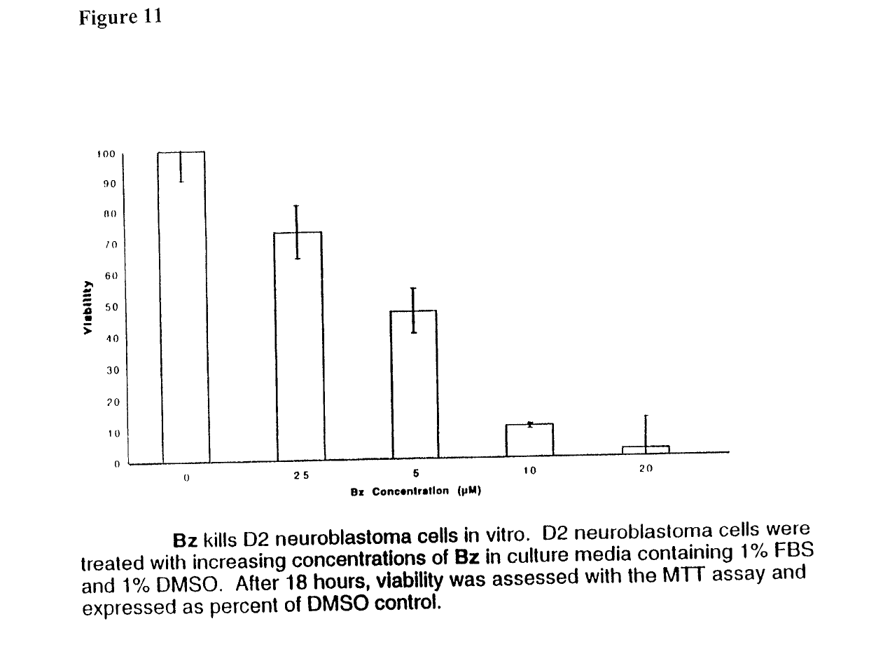

In vitro testing of the D2 cells was conducted to determine their sensitivity to benzodiazepine. D2 cells were plated into 96-well tissue culture plates at a density of 10,000 cells per well in culture media (DMEM, 10% V:V heat inactivated fetal bovine serum (FBS), 100 μml penicillin, 100 μml streptomycin, 290 μml glutamine) and cultured (37° C., 5% CO₂) overnight. Subsequently, culture media was exchanged with media containing 1% FBS. Solvent control (dimethyl sulfoxide (DMSO); final concentration 1% V/V) or benzodiazepine at concentrations of 2.5-20 μM was added. After 18 hours cell viability was assessed using the MTT assay as previously described in this application. FIG. 11 demonstrates that benzodiazepine kills D2 cells in a dose-response fashion.

To test the effect of benzodiazepine on neuroblastoma tumor growth, 1×10⁷ D2 cells were aseptically inoculated into the thigh musculature of each of eight six-week old nu/nu female mice (Jackson Labs). Beginning one week after tumor cell inoculation, 4 mice were dosed with DMSO (20 injected into the peritoneal cavity every day) and 4 mice were dosed with benzodiazepine (2.5 mg dissolved in 20 μl DMSO injected in the peritoneal cavity every day). The mice were evaluated regularly for tumor development and once present the size of the primary tumor was measured every other day. Table 9 demonstrates that in mice that formed tumors, treatment with benzodiazepine significantly decreased the rate of tumor growth.

TABLE 7

| Treatment and control | Days for tumor volume |
|---|---|
| mice with tumors | to increase 5 fold |
| Mouse 1 with Bz-423 | 9 |
| Mouse 2 with Bz-423 | 12 |
| Mouse 3 with Bz-423 | 9 |
| Mouse 4 with Bz-423 | 16 |
| Mouse 5 with DMSO 2 | 3 |
| Mouse 6 with DMSO 3 | 3 |

Administration of Benzodiazepine slows rate of neuroblastoma tumor growth in nu/nu mice (p < 0.02).

Specifically, tumors in control mice increased in volume 5-fold over an average 4 day period, whereas 12 days were required for the same increase in tumor size in benzodiazepine-treated animals (p<0.02). These findings support the claim that benzodiazepine is able to treat human malignant disease in a mouse model. Further, benzodiazepine has specific activity against human neuroblastoma both in vitro and in vivo.

Example 18.5

In another line of experiments, we sought to determine if benzodiazepine is able to kill tumor cells that are otherwise resistant to present standard chemotherapy drugs.

Ovarian cancer provides an excellent model for studying the problem of chemoresistance in that treatment failures are commonly ascribed to the emergence of chemotherapy resistant cells. The A2780 human ovarian cancer cell line is known to contain wild-type p53; express low levels of bcl-2 and bcl-X_{L} survival factors; and is sensitive to treatment with cis-platinum(H) diamine dichloride (CDDP), a standard chemotherapeutic for treatment of ovarian cancer. These cells were transfected with an expression vector encoding human bcl-X_{L}, a survival factor that when over-expressed is linked to the development of chemotherapy resistance. These transfected cells are designated 2B1, and the empty vector transfected controls are designated vector only. A third ovarian cancer cell line, designated SKOV3, was also obtained. This cell line is characterized as: 1. Deficient in wild-type p53 expression; 2. Expressing high levels of endogenous bcl-X_{L}; and 3. Relatively resistant to the cytotoxic actions of CDDP.

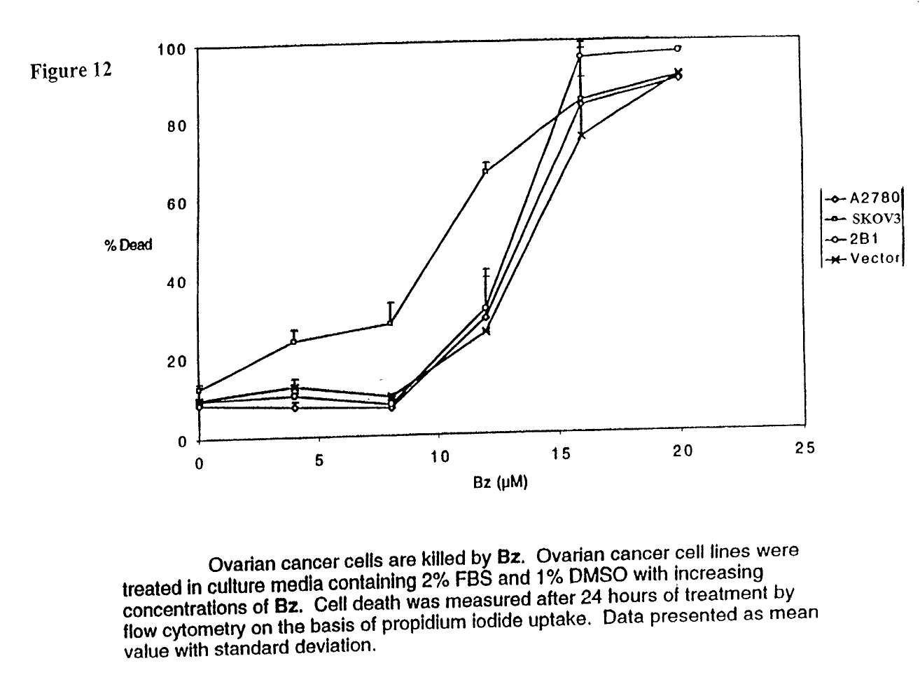

Each of these cell lines was maintained using standard tissue culture conditions in complete media composed of RPNII, 10% FBS, 100 U/ml penicillin, 100 μml streptomycin, 290 μml glutamine. Each cell type was plated into a series of separate wells on 24-well tissue culture plates at 50,000 cells per well. Approximately 24 hours after plating, media was exchanged to contain the same culture media made with only 2% FBS. At this point either control solvent (DMSO, 1% V/V), increasing concentrations of Bz-423 (4-20 μM), or increasing concentrations of CDDP (6.7-66.7 μM) was added to cells. After twenty-four hours of culture all cells present in each well were removed using trypsin-EDTA and mixed with propidium iodide (final concentration 1 μml). After incubating 20 minutes cells were analyzed by flow cytometry (Coulter FACS Calibur) to determine cell death on the basis of plasma membrane integrity measured as the fraction of cells that had taken-up propidiumiodide. Experiments demonstrate that the predicted pattern of chemosensitivity and resistance towards CDDP (A2780 and vector sensitive; 2B1 and SKOV3 resistant) was observed. FIG. 12 demonstrates that benzodiazepine kills each of these types, irrespective of CDDP resistance. Further, benzodiazepine kills ovarian cancer cells that are resistant to standard chemotherapy. Further, benzodiazepine kills tumor cells that express high levels of survival factors (bcl-X_{L}), as well as those that are deficient in p53 expression.

All publications and patents mentioned in the above specification are herein incorporated by reference. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method for regulating cell death comprising:
a. providing:
i. target cells having mitochondria;
ii. an agent that binds to oligomycin sensitivity conferring protein; and
b. exposing said cells to said agent under conditions such that said agent binds to said oligomycin sensitivity conferring protein so as to increase superoxide levels or alter cellular ATP levels in said cells.

2. The method of claim 1, wherein said target cells are in vitro cells.

3. The method of claim 1, wherein said target cells are in vivo cells.

4. The method of claim 1, wherein said target cells are ex vivo cells.

5. The method of claim 1, wherein said target cells are cancer cells.

6. The method of claim 1, wherein said target cells are selected from the group consisting of B cells, T cells, and granulocytes.

7. The method of claim 1, wherein said agent comprises benzodiazepine or a benzodione derivative.

8. The method of claim 1, wherein said agent comprises BZ-432.

9. The method of claim 1, wherein said exposing step results in an increase in cell death of said target cells.

10. A method for inhibiting proliferation in cells comprising:
a. providing:
i. proliferating target cells having mitochondria;
ii. an agent that binds to mitochondrial ATP synthase complex; and
b. exposing said cells to said agent under conditions such that said agent binds to said mitochondrial ATP synthase complex so as to increase superoxide levels or alter cellular ATP levels in said cells.

11. The method of claim 10, wherein said agent binds to oligomycin sensitivity conferring protein.

12. The method of claim 10, wherein said target cells are in vitro cells.

13. The method of claim 10, wherein said target cells are in vivo cells.

14. The method of claim 10, wherein said target cells are ex vivo cells.

15. The method of claim 10, wherein said target cells are cancer cells.

16. The method of claim 10, wherein said target cells are selected from the group consisting of B cells, T cells, and granulocytes.

17. The method of claim 10, wherein said target cells are proliferating cells.

18. The method of claim 10, wherein said agent comprises benzodiazepine or a benzodione derivative.

19. The method of claim 10, wherein said agent comprises BZ-432.

20. A pharmaceutical composition comprising:
a. a sufficient dose of an agent that binds to oligomycin sensitivity conferring protein so as to increase superoxide or alter cellular ATP levels in cells of a subject exposed to said agent; and
b. instructions for using said agent for treating a condition that is ameliorated by initiation of cell death.

21. The composition of claim 20, wherein said agent comprises benzodiazepine or a benzodione derivative.

22. The composition of claim 20, wherein said agent comprises BZ-432.

23. The composition of claim 20, wherein said condition is cancer.

24. The composition of claim 20, wherein said condition is selected from the group consisting of proliferative diseases and autoimmune diseases.

25. The composition of claim 20, wherein said condition is selected from the group consisting of graft-versus-host disease and transplant rejection.

26. A pharmaceutical composition comprising:
a. a sufficient dose of an agent that binds to mitochondrial ATP synthase complex so as to increase superoxide or alter cellular ATP levels in cells of a subject exposed to said agent; and
b. instructions for using said agent for treating an autoimmune disease, a proliferative disease, or cancer.

27. The composition of claim 26, wherein said agent comprises benzodiazepine or a benzodione derivative.

28. The composition of claim 26, wherein said agent comprises BZ-432.

29. A method for identifying agents useful for treating proliferative diseases, autoimmune diseases, or cancer comprising:
a. providing:
i. mitochondrial ATP synthase complex;
ii. benzodiazepine or a benzodione derivative; and
iii. a candidate agent; and
b. exposing said mitochondrial ATP synthase complex to said benzodiazepine or a benzodione derivative and said candidate agent; and
c. comparing the binding of said benzodiazepine or a benzodione derivative and said candidate agent to said mitochondrial ATP synthase complex.

30. The method of claim 29, wherein said comparing comprises observing cell death, growth rate, or cell number in cells containing said mitochondrial ATP synthase complex.

31. The method of claim 29, wherein said comparing comprises measuring superoxide levels in cells containing said mitochondrial ATP synthase complex.

32. The method of claim 29, wherein said comparing comprises measuring binding affinities of said benzodiazepine or a benzodione derivative and said candidate agent to said mitochondrial ATP synthase complex.

33. The method of claim 29, wherein said comparing comprises detecting binding of said candidate agent to oligomycin sensitivity conferring protein.

34. A method for identifying pharmaceutical agents, comprising:
a. providing an agent that binds to mitochondrial ATP synthase complex so as to generate superoxide free radicals, initiate cell death, or alter cellular proliferation;
b. chemically modifying said agent to generate a library of candidate pharmaceutical agents; and
c. selecting one or more individual members of said library of candidate agents based on their increased ability to generate superoxide free radicals, initiate cell death, or alter cellular proliferation compared to said agent.

35. The method of claim 34, further comprising the step of testing said one or more individual members of said library for toxicity in a tissue or animal.

36. The method of claim 34, further comprising the step of submitting said one or more individual members of said library to a regulatory agency for approval as a commercial product.

37. A method for screening for agents that selectively induce cell death or inhibit the growth or proliferation of activated cells, comprising:
a. providing:
i. a first cell sample comprising at least one unactivated cell;
ii. a second cell sample comprising at least one unactivated cell;
iii. a third cell sample comprising at least one unactivated cell;
iv. an effective amount of an activating agent;
v. an effective amount of a candidate agent;
vi. an effective ratio and amount of said activating agent and said candidate agent; and
b. contacting said first cell sample with said effective amount of said activating agent;
c. contacting said second cell sample with said effective amount of said candidate agent;
d. contacting said third cell sample with said effective ratio and amount of said activating agent and said candidate agent;
e. comparing the level of cell death or cell number in said third cell sample to the level of cell death or cell number in said first cell sample and said second cell sample; and
f. comparing the amount of cell death or growth inhibition in said third cell sample to the level of cell death or growth inhibition in said first cell sample and said second cell sample.

38. The method of claim 37, further comprising step g) selecting a candidate agent contacted to said third sample if said level of cell death or growth inhibition in said third cell sample is greater than said cell death in said first cell sample and said second cell sample.

39. The method of claim 37, wherein said first, second, and third samples comprise a B cell, a T cell, a granulocyte, or a cancer cell.

40. The method of claim 37, wherein said contacting is in vitro.

41. The method of claim 37, wherein said contacting is in vivo.

42. The method of claim 37, wherein said activating agent is selected from the group consisting of a T cell ligand, BAFF ligand, TNF, Fas ligand (FasL), Toll receptor, APRIL receptor, CD40 ligand, cytokines, chemokines, hormones, steroids, a B cell ligand, gamma irradiation, UV irradiation, an agent or condition that enhances cell stress, and antibodies that specifically recognize and bind cell surface receptors.

43. The method of claim 42, wherein said antibody is selected from the group consisting of anti-CD4, anti-CD8, anti-CD20, anti-BAFF, anti-TNF, anti-CD40, anti-CD3, anti-CD28, anti-B220, anti-Toll receptor, anti-APRIL receptor, anti-B cell receptor, and anti-T cell receptor.

44. A method for inhibiting induced cell death in an activated target cell by contacting the activated target cell with an effective amount of an agent that inhibits the formation of superoxide in said activated target cell prior to mitochondrial permeability transition.

45. The method of claim 44, wherein said activated target cell is in vitro.

46. The method of claim 44, wherein said activated target cell is in vivo.

47. The method of claim 44, wherein said agent binds to mitochondrial ATP synthase.

48. The method of claim 44, wherein said agent binds to oligomycin sensitivity conferring protein.

49. The method of claim 44, wherein said agent comprises benzodiazepine or a benzodione derivative.

50. A method for screening for agents that selectively induce cell death or inhibit the growth or proliferation of activated cells, comprising:
a. providing:
i. a first cell sample comprising at least one unactivated cell;
ii. a second cell sample comprising at least one unactivated cell;
iii. a candidate agent; and
b. contacting said first cell sample with said candidate agent; and
c. comparing the intracellular concentration of superoxide in first and second cells.

51. The method of claim 50, further comprising step d) selecting said candidate agent contacted to said first cell sample if the intracellular concentration of superoxide is greater in said first cell sample than in said second cell sample.

52. The method of claim 51, further comprising steps of:
e) providing:
i) an agent known to increase superoxide levels in treated unactivated cells;
ii) a third cell sample comprising at least one unactivated cell;
iii) a fourth cell sample comprising at least one unactivated cell; and
f) treating said third cell sample with said agent known to increase superoxide levels;
g) treating said fourth cell sample with said with said agent known to increase superoxide levels and said selected candidate agent; and
h) identifying whether or not said candidate agent synergistically increases superoxide levels with said agent known to increase superoxide levels by determining whether superoxide levels are higher in said treated fourth cell sample as compared to said treated third cell sample.

53. A pharmaceutical cocktail comprising the agent known to increase superoxide levels and said identified candidate agent of claim 52.
